(19) United States
(12) Patent Application Publication
(10) Pub. No.: US 2024/0327869 A1
(43) Pub. Date: Oct. 3, 2024

(54) SYNTHETIC GENETIC ELEMENTS FOR BIOMANUFACTURE

(71) Applicant: JANSSEN BIOTECH, INC., Horsham, PA (US)

(72) Inventors: William Lloyd PERRY, III, Warrington, PA (US); Brian TOMKOWICZ, North Wales, PA (US)

(73) Assignee: JANSSEN BIOTECH, INC., Horsham, PA (US)

(21) Appl. No.: 18/744,565

(22) Filed: Jun. 14, 2024

Related U.S. Application Data
(62) Division of application No. 16/934,970, filed on Jul. 21, 2020, now Pat. No. 12,049,638.
(60) Provisional application No. 62/877,577, filed on Jul. 23, 2019.
(60) Provisional application No. 62/877,561, filed on Jul. 23, 2019.
(60) Provisional application No. 62/877,551, filed on Jul. 23, 2019.
(60) Provisional application No. 62/877,540, filed on Jul. 23, 2019.
(60) Provisional application No. 62/877,532, filed on Jul. 23, 2019.
(60) Provisional application No. 62/877,524, filed on Jul. 23, 2019.
(60) Provisional application No. 62/877,516, filed on Jul. 23, 2019.
(60) Provisional application No. 62/877,508, filed on Jul. 23, 2019.

(51) Int. Cl. C12N 15/86; C12N 7/00

(52) U.S. Cl. CPC C12N 15/86; C12N 7/00; C12N 2750/14121; C12N 2750/14151; C12N 2800/50; C12N 2830/36; C12N 2830/40; C12N 2830/42; C12N 2830/50

(57) ABSTRACT

Recombinant constructs, cells and means for improved production of Adeno-Associated Viruses (AAVs) are described. Also described are methods of using the constructs and cells to produce recombinant AAVs.

This application is a divisional of U.S. application Ser. No. 16/934,970, filed Jul. 21, 2020, which claims the benefit of U.S. Provisional Application No. 62/877,508 filed Jul. 23, 2019; U.S. Provisional Application No. 62/877,516 filed Jul. 23, 2019; U.S. Provisional Application No. 62/877,524 filed Jul. 23, 2019; U.S. Provisional Application No. 62/877,532 filed Jul. 23, 2019; U.S. Provisional Application No. 62/877,540 filed Jul. 23, 2019; U.S. Provisional Application No. 62/877,551 filed Jul. 23, 2019; U.S. Provisional Application No. 62/877,561 filed Jul. 23, 2019; and U.S. Provisional Application No. 62/877,577 filed Jul. 23, 2019, the disclosure of each of which is incorporated by reference herein in its entirety.

### REFERENCE TO SEQUENCE LISTING SUBMITTED ELECTRONICALLY

This application contains an electronic Sequence Listing which has been submitted in XML format via Patent Center, the entire content of which is incorporated by reference herein in its entirety. The Sequence Listing XML file submitted via Patent Center is entitled “14620-744-999_SEQ_LISTING.xml”, was created on Jun. 8, 2024, and is 182,370 bytes in size.

### BACKGROUND

Adeno-Associated Virus (AAV) has a linear single-stranded DNA (ssDNA) genome with two inverted terminal repeats (ITR) at the termini. The ITRs flank the two viral genes—rep (replication) and cap (capsid), which encode non-structural and structural proteins, respectively. The rep gene encodes four regulatory proteins Rep78, Rep68, Rep52 and Rep40, through the use of two promoters and alternative splicing. More specifically, Rep78 and Rep68 are transcribed from the P5 promoter and Rep 40 and Rep52 are transcribed from the P19 promoter (which is embedded within the Rep78 and Rep68 reading frame). The P5 and P19 promoters are activated by the adenovirus E1A gene and are active in cells such as HEK293 that was transformed using the adenovirus E1 genes. These Rep proteins are involved in AAV genome replication. The cap gene, through alternative splicing and initiation of translation, gives rise to three capsid proteins, VP1 (virion protein 1), VP2 and VP3, which assemble into a near-spherical protein shell of the virus. The AAV virus does not encode a polymerase, thus relying on cellular polymerases for genome replication.

Large-scale production of AAV in mammalian cells may be possible if the AAV rep and cap genes could be stably integrated or maintained in the cells and later induced to produce AAV in high density cultures. However, the expression of Rep proteins can be cytotoxic or cytostatic to the host cells, making it difficult to develop stable cell lines in hosts where rep genes are expressed such as those that express the Adenovirus E1 genes such as HEK293 cells. Because AAV encodes four Rep proteins with overlapping reading frames that result from the use of two promoters and alternate splicing, the use of an inducible promoter to control rep gene expression is not straightforward.

The cytotoxic or cytostatic nature of the four Rep proteins has prevented the development of stable cell lines that can produce high-titer AAV using native rep/cap promoters (Clark et al. (1995) Hum. Gene Ther. 6:1329-1341; Chadeuf et al. (2000) J. Gene med. 2:260-268). Several groups have attempted to regulate Rep expression recombinantly. Yang replaced the P5 promoter with the mouse metallothionein promoter. While stable clones in HEK293 demonstrated metal-inducible rep78 expression, rep30 and rep42 expression (driven by the internal P19 promoter) was only detected at low levels and the growth rate of the cells was substantially decreased (Yang et al. (1994) J. Virol 68: 4847-4856). Ogasawara replaced the P5 promoter with a ubiquitous promoter containing a loxP flanked stuffer that could be activated by Cre recombinase. Neither rep52, rep40, or cap genes were induced in stable clones infected with Adenovirus-Cre, which suggested constitutive rep52/rep40 expression was also harmful to cells (Ogasawara et al. (1999) J. Gen. Virol. 80: 2477-2480).

Another approach to regulated rep expression was described by Xiao and coworkers (Qiao et al. (2002) J. Virol. 76: 13015-13027; Yuan et al. (2011) Hum. Gene Ther. 22:613-624). Xiao inserted an artificial intron into the rep gene in a coding region all four Rep proteins share and inserted a loxP flanked stop cassette containing the poly(A) sequences alone or in combination with puro, the puromycin resistance gene, into the intron. Expression of all the Rep proteins is inhibited, allowing stable cell lines in HEK293 cells to be generated. Delivery of Cre recombinase (by adenovirus infection) into the cell excises the stop cassette through recombining the loxP sites, allowing full length pre-mRNA to be transcribed. The remaining intron sequence is then precisely removed by RNA splicing, restoring the coding sequence for all four Rep proteins, and, thus, initiating the production of AAV from an integrated ITR-flanked transgene. However, because Cre recombinase recognizes two identical loxP sites, the loxP sites remain identical after recombination, thus additional recombination may be possible since Cre catalyzes both joining and excision reactions.

AAV rep genes are only expressed in cells that also express the adenovirus E1 (Early region 1) genes. Several stable rep/cap cell lines have been constructed in hosts that do not express the E1 genes including HeLa (Clark et al (1995) Hum. Gen. Therap. 6: 1329-1341; Yang et al. (1994) J. Virol. 68: 4847-4856; Gao et. Al (1998) Hu, Gen. Ther. 9: 2353-2362), A549 (Gao et al. (2002) Mol Ther. 5:644-659), and Vero (Beal et al. (2007)₁₀th Annual Meeting of American Society of Gene Therapy, Seattle, WA, May 30-Jun. 3, 2007). The largest drawback to these cell lines is that an E1-intact (and usually replication competent) adenovirus is required for AAV production, which may pose increased safety risks as a contaminant of AAV virus preps.

AAV production systems have been described using several different viruses to provide helper functions and to deliver the recombinant transgene and/or AAV genes to human cells including Herpes (Thomas et al. (2009) Hum Gene Ther. 20:861-70; Clement et al. (2009) Hum Gene Ther. 20:796-806), Vaccinia virus (Wang et al. (2017) Mol. Ther. Methods Clin Devel. 7: 146-155.), and Adenovirus (Fisher et al. (1996) Hum gene Ther. 7: 2079-2087; Gao et al. (1998) Hum Gene Ther. 2353-2362. Liu et al. (1999) Gene Ther 6: 293-299). These approaches require production of several different viruses (and in some cases recombinant host cell lines). AAV has also been produced in insect cells using baculoviruses (Mietzsch et al. (2014) Hum Gene Ther. 25:212-22; Aslanidi et al. (2009) Proc Natl Acad Sci USA. 106:5059-5064; Cecchini et al. (2011) Hum Gene Ther. 22:1021-1030). Whether AAVs produced in insect versus human cells are functionally equivalent is still an open question.

There is a need for improved production of AAVs with recombinant constructs and cells.

### SUMMARY

In one aspect, provided herein is a non-naturally occurring nucleic acid molecule comprising a modified adeno-associated virus (AAV) rep gene having an AAV rep gene encoding four Rep proteins Rep78, Rep68, Rep52 and Rep40 and an artificial intron inserted into a coding sequence of the rep gene shared by the four Rep proteins, wherein the artificial intron comprises a stop cassette inserted downstream of the 5′ splice site and upstream of the branch site of the artificial intron, and the stop cassette comprises, in 5′ to 3′ order: (a) an attP site having a nucleotide sequence at least 90%, 91%, 92%, 93%, 94%, 95%, 96%, 97%, 98%, 99% or 100%, identical to SEQ ID NO:7, preferably, an attP site having the nucleotide sequence of SEQ ID NO:7; (b) a splice acceptor; (c) a terminator; and (d) an attB site having a nucleotide sequence at least 90%, 91%, 92%, 93%, 94%, 95%, 96%, 97%, 98%, 99% or 100%, identical of SEQ ID NO:8 or SEQ ID NO:9, preferably, an attB site having the nucleotide sequence of SEQ ID NO:8 or SEQ ID NO:9.

In one embodiment, the splice acceptor comprises the nucleotide sequence of SEQ ID NO:17.

In one embodiment, the terminator comprises a polyadenylation signal. In one embodiment, the terminator further comprises the nucleotide sequence of SEQ ID NO:19.

In one embodiment, the stop cassette comprises a gene encoding a selectable marker, preferably a neomycin phosphotransferase expression cassette having the nucleotide sequence of SEQ ID NO:18.

In one embodiment, the artificial intron comprises, in 5′ to 3′ order, the nucleotide sequence of SEQ ID NO:14, the stop cassette, and the nucleotide sequence of SEQ ID NO:15.

In one embodiment, the AAV rep gene comprises a rep gene of one of AAV1 to AAV8, or a hybrid thereof. In one embodiment, the AAV rep gene comprises the rep gene of human AAV2 having nucleotide numbers 190 to 2202 of the nucleotide sequence of GenBank accession number NC_001401.2. In one embodiment, the artificial intron is inserted between nucleotide numbers 996 to 1905 of the nucleotide sequence of GenBank accession number NC_001401.2.

In one embodiment, the artificial intron is inserted immediately downstream of nucleotide number 1052, 1061, 1712, 1906, 1022, 1112, 1475, 1514, 1700, 1742, 1784 or 1340, preferably nucleotide number 1052, of the nucleotide sequence of GenBank accession number NC_001401.2.

In one aspect, provided herein is a non-naturally occurring nucleic acid molecule comprising a modified AAV rep gene comprising, in 5′ to 3′ order: (a) a 5′ portion of an AAV rep gene having the nucleotide sequence of SEQ ID NO:55; (b) an artificial intron comprising, in 5′ to 3′ order: (i) a 5′ intron fragment having the nucleotide sequence of SEQ ID NO:14; (ii) a stop cassette comprising, in 5′ to 3′ order: (1) an attP site having the nucleotide sequence of SEQ ID NO:7; (2) a splice acceptor having the nucleotide sequence of SEQ ID NO:17; (3) a neomycin phosphotransferase expression cassette having the nucleotide sequence of SEQ ID NO:18; (4) a terminator having the nucleotide sequence of SEQ ID NO:19; and (5) an attB site having the nucleotide sequence of SEQ ID NO:8 or SEQ ID NO:9; and (iii) a 3′ intron fragment having the nucleotide sequence of SEQ ID NO: 15; and (c) a 3′ portion of the AAV rep gene having the nucleotide sequence of SEQ ID NO:56.

In one aspect, provided herein is a non-naturally occurring nucleic acid molecule comprising a modified AAV rep gene comprising, in 5′ to 3′ order: (a) a 5′ portion of an AAV rep gene having the nucleotide sequence of SEQ ID NO:73; (b) an artificial intron comprising, in 5′ to 3′ order: (i) a 5′ intron fragment having the nucleotide sequence of SEQ ID NO:14; (ii) a stop cassette comprising, in 5′ to 3′ order: (1) an attP site having the nucleotide sequence of SEQ ID NO:7; (2) a splice acceptor having the nucleotide sequence of SEQ ID NO:17; (3) a neomycin phosphotransferase expression cassette having the nucleotide sequence of SEQ ID NO:18; (4) a terminator having the nucleotide sequence of SEQ ID NO:19; and (5) an attB site having the nucleotide sequence of SEQ ID NO:8 or SEQ ID NO:9; and (iii) a 3′ intron fragment having the nucleotide sequence of SEQ ID NO:66; and (c) a 3′ portion of the AAV rep gene having the nucleotide sequence of SEQ ID NO:56. In one embodiment, the stop cassette comprises the nucleotide sequence of SEQ ID NO:16.

In one embodiment, the non-naturally occurring nucleic acid molecule further includes an AAV cap gene encoding three capsid proteins VP1, VP2 and VP3. In one embodiment, the AAV cap gene comprises a cap gene of one of AAV1 to AAV9 and AAVDJ, or a hybrid thereof. In one embodiment, the AAV cap gene comprises the cap gene of human AAV9 having the nucleotide sequence of GenBank accession number AY530579.1. In one embodiment, the AAV cap gene further comprises a polyadenylation signal, preferably a polyadenylation signal of AAV2 having nucleotide numbers 4411 to 4466 of the nucleotide sequence of GenBank accession number NC_001401.2, and an enhancer, preferably an AAV2 rep P5 promoter having nucleotide numbers 190 to 313 of the nucleotide sequence of GenBank accession number NC_001401.2, wherein the polyadenylation signal and the enhancer are both downstream of the coding sequence of the cap gene. In one embodiment, the non-naturally occurring nucleic acid molecule further includes a transgene flanked by a pair of AAV inverted terminal repeats (ITRs) downstream of the AAV cap gene.

In one embodiment, the non-naturally occurring nucleic acid molecule still further includes a first insulator upstream of the modified AAV rep gene and optionally a second insulator downstream of the transgene flanked by the ITRs, preferably, the first insulator and the second insulator are independently selected from the group consisting of: (a) a human anti-repressor element 40 having the nucleotide sequence of SEQ ID NO:24; (b) a mouse anti-repressor element 40 having the nucleotide sequence of SEQ ID NO:25; (c) an anti-repressor element 04 having the nucleotide sequence of GenBank accession number AY190749.1; (d) an anti-repressor element 06 having the nucleotide sequence of GenBank accession number AY190750.1; (e) an anti-repressor element 07 having the nucleotide sequence of GenBank accession number AY190751.1; (f) an anti-repressor element 12 having the nucleotide sequence of GenBank accession number AY190752.1; (g) an anti-repressor element 13 having the nucleotide sequence of GenBank accession number AY190753.1; (h) an anti-repressor element 35 having the nucleotide sequence of GenBank accession number AY190754.1; (i) an anti-repressor element 36 having the nucleotide sequence of GenBank accession number AY190755.1; (j) an anti-repressor element 52 having the nucleotide sequence of GenBank accession number AY190757.1; (k) an anti-repressor element 53 having the nucleotide sequence of GenBank accession number AY190758.1; and (l) a Chicken HS4 insulator from the globin locus having the nucleotide sequence of AY040835.1 in two or more copies, more preferably, the first insulator and the second insulator have the nucleotide sequences of SEQ ID NO:24 and SEQ ID NO:25, respectively. In one embodiment, the non-naturally occurring nucleic acid molecule comprises the first insulator upstream of the modified AAV rep gene, and further comprises a first spacer sequence and a second spacer sequence upstream and downstream of the transgene, respectively, wherein the first spacer sequence and the second spacer sequence are independently selected from the group consisting of: (a) a nucleotide sequence of SEQ ID NO:67; and (b) a nucleotide sequence of SEQ ID NO:68. In one embodiment, the ITR has the nucleotide sequence of SEQ ID NO:20, the transgene comprises a promoter operably linked to a coding sequence, and the coding sequence is operably linked a polyadenylation signal; preferably, the promoter has the nucleotide sequence of SEQ ID NO:21 and the polyadenylation signal has the nucleotide sequence SEQ ID NO:23.

In one aspect, provided herein is a non-naturally occurring nucleic acid molecule comprising, in 5′ to 3′ order: (A) a first insulator, preferably the first insulator has the nucleotide sequence of SEQ ID NO:24; (B) a modified AAV rep gene comprising, in 5′ to 3′ order: (i) a 5′ portion of an AAV rep gene, preferably the 5′ portion of the AAV rep gene has the nucleotide sequence of SEQ ID NO:55; (ii) an artificial intron comprising, in 5′ to 3′ order: (a) a 5′ intron fragment, preferably the 5′ intron fragment has the nucleotide sequence of SEQ ID NO:14; (b) a stop cassette comprising, in 5′ to 3′ order: (1) an attP site having the nucleotide sequence of SEQ ID NO:7; (2) a splice acceptor, preferably the splice acceptor has the nucleotide sequence of SEQ ID NO:17; (3) a gene encoding a selectable marker, preferably a neomycin phosphotransferase expression cassette having the nucleotide sequence of SEQ ID NO:18; (4) a terminator, preferably the terminator has the nucleotide sequence of SEQ ID NO:19; and (5) an attB site having the nucleotide sequence of SEQ ID NO:8 or SEQ ID NO:9; and (c) a 3′ intron fragment, preferably the 3′ intron fragment has the nucleotide sequence of SEQ ID NO:15; (iii) a 3′ portion of the AAV rep gene, preferably the 3′ portion of the AAV rep gene has the nucleotide sequence of SEQ ID NO:56; (C) an AAV cap gene, preferably the AAV cap gene comprises the nucleotide sequence of SEQ ID NO:57; (D) a transgene flanked by a pair of AAV ITRs, preferably, the AAV ITR has the nucleotide sequence of SEQ ID NO:20, and the transgene comprises a promoter operably linked to a coding sequence, and the coding sequence is operably linked a polyadenylation signal; more preferably, the promoter has the nucleotide sequence of SEQ ID NO:21 and the polyadenylation signal has the nucleotide sequence SEQ ID NO:23; and (E) a second insulator, preferably the second insulator has the nucleotide sequence of SEQ ID NO:25.

In one aspect, provided herein is a non-naturally occurring nucleic acid molecule comprising, in 5′ to 3′ order: (A) a first insulator, preferably the first insulator has the nucleotide sequence of SEQ ID NO:24; (B) a modified AAV rep gene comprising, in 5′ to 3′ order: (i) a 5′ portion of an AAV rep gene, preferably the 5′ portion of the AAV rep gene has the nucleotide sequence of SEQ ID NO:73; (ii) an artificial intron comprising, in 5′ to 3′ order: (a) a 5′ intron fragment, preferably the 5′ intron fragment has the nucleotide sequence of SEQ ID NO:14; (b) a stop cassette comprising, in 5′ to 3′ order: (1) an attP site having the nucleotide sequence of SEQ ID NO:7; (2) a splice acceptor, preferably the splice acceptor has the nucleotide sequence of SEQ ID NO:17; (3) a gene encoding a selectable marker, preferably a neomycin phosphotransferase expression cassette having the nucleotide sequence of SEQ ID NO:18; (4) a terminator, preferably the terminator has the nucleotide sequence of SEQ ID NO:19; and (5) an attB site having the nucleotide sequence of SEQ ID NO:8 or SEQ ID NO:9; and (c) a 3′ intron fragment, preferably the 3′ intron fragment has the nucleotide sequence of SEQ ID NO:66; (iii) a 3′ portion of the AAV rep gene, preferably the 3′ portion of the AAV rep gene has the nucleotide sequence of SEQ ID NO:56; (C) an AAV cap gene; (D) a transgene flanked by (1) a pair of AAV ITRs, preferably, the AAV ITR has the nucleotide sequence of SEQ ID NO:20, and the transgene comprises a promoter operably linked to a coding sequence, and the coding sequence is operably linked a polyadenylation signal; more preferably, the promoter has the nucleotide sequence of SEQ ID NO:21 and the polyadenylation signal has the nucleotide sequence SEQ ID NO:23; and (2) a pair of spacer sequences, preferably, the spacer sequences have a nucleotide sequence of SEQ ID NO:67 and SEQ ID NO:68.

In one aspect, provided herein is a vector comprising a non-naturally occurring nucleic acid molecule described above; preferably, the vector is a plasmid; more preferably, the plasmid comprises the nucleotide sequence of SEQ ID NO:12.

In one aspect, provided herein is a vector comprising a non-naturally occurring nucleic acid molecule described above; preferably, the vector is a plasmid; more preferably, the plasmid comprises the nucleotide sequence of SEQ ID NO:70.

In one aspect, provided herein is a method of making the non-naturally occurring nucleic acid molecule of described above. In specific embodiments, provided herein is a method of making the vector comprising a non-naturally occurring nucleic acid molecule described above; preferably, the vector is a plasmid; more preferably, the plasmid comprises the nucleotide sequence of SEQ ID NO:12. In another embodiment, provided herein is a method of making the vector comprising a non-naturally occurring nucleic acid molecule described above; preferably, the vector is a plasmid; more preferably, the plasmid comprises the nucleotide sequence of SEQ ID NO:70.

In one aspect, provided herein is a cell comprising a non-naturally occurring nucleic acid molecule comprising a modified adeno-associated virus (AAV) rep gene having an AAV rep gene encoding four Rep proteins Rep78, Rep68, Rep52 and Rep40 and an artificial intron inserted into a coding sequence of the rep gene shared by the four Rep proteins, wherein the artificial intron comprises a stop cassette inserted downstream of the 5′ splice site and upstream of the branch site of the artificial intron, and the stop cassette comprises, in 5′ to 3′ order: (a) an attP site having a nucleotide sequence at least 90%, 91%, 92%, 93%, 94%, 95%, 96%, 97%, 98%, 99% or 100%, identical to SEQ ID NO:7, preferably, an attP site having the nucleotide sequence of SEQ ID NO:7; (b) a splice acceptor; (c) a terminator; and (d) an attB site having a nucleotide sequence at least 90%, 91%, 92%, 93%, 94%, 95%, 96%, 97%, 98%, 99% or 100%, identical of SEQ ID NO:8 or SEQ ID NO:9, preferably, an attB site having the nucleotide sequence of SEQ ID NO:8 or SEQ ID NO:9.

In one embodiment, the splice acceptor comprises the nucleotide sequence of SEQ ID NO:17.

In one embodiment, the terminator comprises a polyadenylation signal. In one embodiment, the terminator further comprises the nucleotide sequence of SEQ ID NO:19.

In one embodiment, the stop cassette comprises a gene encoding a selectable marker, preferably a neomycin phosphotransferase expression cassette having the nucleotide sequence of SEQ ID NO:18.

In one embodiment, the artificial intron comprises, in 5′ to 3′ order, the nucleotide sequence of SEQ ID NO:14, the stop cassette, and the nucleotide sequence of SEQ ID NO:15. In another embodiment, the artificial intron comprises, in 5′ to 3′ order, the nucleotide sequence of SEQ ID NO:14, the stop cassette, and the nucleotide sequence of SEQ ID NO:66.

In one embodiment, the AAV rep gene comprises a rep gene of one of AAV1 to AAV8, or a hybrid thereof. In one embodiment, the AAV rep gene comprises the rep gene of human AAV2 having nucleotide numbers 190 to 2202 of the nucleotide sequence of GenBank accession number NC_001401.2. In one embodiment, the artificial intron is inserted between nucleotide numbers 996 to 1905 of the nucleotide sequence of GenBank accession number NC_001401.2. In one embodiment, the artificial intron is inserted immediately downstream of nucleotide number 1052, 1061, 1712, 1906, 1022, 1112, 1475, 1514, 1700, 1742, 1784 or 1340, preferably nucleotide number 1052, of the nucleotide sequence of GenBank accession number NC_001401.2.

In one aspect, provided herein is a cell comprising a non-naturally occurring nucleic acid molecule comprising a modified AAV rep gene comprising, in 5′ to 3′ order: (a) a 5′ portion of an AAV rep gene having the nucleotide sequence of SEQ ID NO:55; (b) an artificial intron comprising, in 5′ to 3′ order: (i) a 5′ intron fragment having the nucleotide sequence of SEQ ID NO:14; (ii) a stop cassette comprising, in 5′ to 3′ order: (1) an attP site having the nucleotide sequence of SEQ ID NO:7; (2) a splice acceptor having the nucleotide sequence of SEQ ID NO:17; (3) a neomycin phosphotransferase expression cassette having the nucleotide sequence of SEQ ID NO:18; (4) a terminator having the nucleotide sequence of SEQ ID NO:19; and (5) an attB site having the nucleotide sequence of SEQ ID NO:8 or SEQ ID NO:9; and (iii) a 3′ intron fragment having the nucleotide sequence of SEQ ID NO:15; and (c) a 3′ portion of the AAV rep gene having the nucleotide sequence of SEQ ID NO:56.

In one aspect, provided herein is a cell comprising a non-naturally occurring nucleic acid molecule comprising a modified AAV rep gene comprising, in 5′ to 3′ order: (a) a 5′ portion of an AAV rep gene having the nucleotide sequence of SEQ ID NO:73; (b) an artificial intron comprising, in 5′ to 3′ order: (i) a 5′ intron fragment having the nucleotide sequence of SEQ ID NO:14; (ii) a stop cassette comprising, in 5′ to 3′ order: (1) an attP site having the nucleotide sequence of SEQ ID NO:7; (2) a splice acceptor having the nucleotide sequence of SEQ ID NO:17; (3) a neomycin phosphotransferase expression cassette having the nucleotide sequence of SEQ ID NO:18; (4) a terminator having the nucleotide sequence of SEQ ID NO:19; and (5) an attB site having the nucleotide sequence of SEQ ID NO:8 or SEQ ID NO:9; and (iii) a 3′ intron fragment having the nucleotide sequence of SEQ ID NO:66; and (c) a 3′ portion of the AAV rep gene having the nucleotide sequence of SEQ ID NO:56.

In one embodiment, the stop cassette comprises the nucleotide sequence of SEQ ID NO:16.

In one embodiment, the cell described above further includes an AAV cap gene encoding three capsid proteins VP1, VP2 and VP3. In one embodiment, the AAV cap gene comprises a cap gene of one of AAV1 to AAV9 and AAVDJ, or a hybrid thereof. In one embodiment, the AAV cap gene comprises the cap gene of human AAV9 having the nucleotide sequence of GenBank accession number AY530579.1. In one embodiment, the AAV cap gene comprises the cap gene of a hybrid of AAV9.

In one embodiment, the AAV cap gene further comprises a polyadenylation signal, preferably a polyadenylation signal of AAV2 having nucleotide numbers 4411 to 4466 of the nucleotide sequence of GenBank accession number NC_001401.2, and an enhancer, preferably an AAV2 rep P5 promoter having nucleotide numbers 190 to 313 of the nucleotide sequence of GenBank accession number NC_001401.2, wherein the polyadenylation signal and the enhancer are both downstream of the coding sequence of the cap gene.

In one embodiment, the cell comprising a cap gene further includes a transgene flanked by a pair of AAV inverted terminal repeats (ITRs) downstream of the AAV cap gene. In one embodiment, the cell further includes a first insulator upstream of the modified AAV rep gene and optionally a second insulator downstream of the transgene flanked by the ITRs, preferably, the first insulator and the second insulator are independently selected from the group consisting of: (a) a human anti-repressor element 40 having the nucleotide sequence of SEQ ID NO:24; (b) a mouse anti-repressor element 40 having the nucleotide sequence of SEQ ID NO:25; (c) an anti-repressor element 04 having the nucleotide sequence of GenBank accession number AY190749.1; (d) an anti-repressor element 06 having the nucleotide sequence of GenBank accession number AY190750.1; (e) an anti-repressor element 07 having the nucleotide sequence of GenBank accession number AY190751.1; (f) an anti-repressor element 12 having the nucleotide sequence of GenBank accession number AY190752.1; (g) an anti-repressor element 13 having the nucleotide sequence of GenBank accession number AY190753.1; (h) an anti-repressor element 35 having the nucleotide sequence of GenBank accession number AY190754.1; (i) an anti-repressor element 36 having the nucleotide sequence of GenBank accession number AY190755.1; (j) an anti-repressor element 52 having the nucleotide sequence of GenBank accession number AY190757.1; (k) an anti-repressor element 53 having the nucleotide sequence of GenBank accession number AY190758.1; and (l) a Chicken HS4 insulator from the globin locus having the nucleotide sequence of AY040835.1 in two or more copies, more preferably, the first insulator and the second insulator have the nucleotide sequences of SEQ ID NO:24 and SEQ ID NO:25, respectively. In one embodiment, the cell comprises the first insulator upstream of the modified AAV rep gene, and further comprises a first spacer sequence and a second spacer sequence upstream and downstream of the transgene, respectively, wherein the first spacer sequence and the second spacer sequence are independently selected from the group consisting of: (a) a nucleotide sequence of SEQ ID NO:67; and (b) a nucleotide sequence of SEQ ID NO:68.

In one embodiment, the ITR has the nucleotide sequence of SEQ ID NO:20, the transgene comprises a promoter operably linked to a coding sequence, and the coding sequence is operably linked a polyadenylation signal; preferably, the promoter has the nucleotide sequence of SEQ ID NO:21 and the polyadenylation signal has the nucleotide sequence SEQ ID NO:23.

In one aspect, provided herein is a cell comprising a non-naturally occurring nucleic acid molecule comprising, in 5′ to 3′ order: (A) a first insulator, preferably the first insulator has the nucleotide sequence of SEQ ID NO:24; (B) a modified AAV rep gene comprising, in 5′ to 3′ order: (i) a 5′ portion of an AAV rep gene, preferably the 5′ portion of the AAV rep gene has the nucleotide sequence of SEQ ID NO:55; (ii) an artificial intron comprising, in 5′ to 3′ order: (a) a 5′ intron fragment, preferably the 5′ intron fragment has the nucleotide sequence of SEQ ID NO:14; (b) a stop cassette comprising, in 5′ to 3′ order: (1) an attP site having the nucleotide sequence of SEQ ID NO:7; (2) a splice acceptor, preferably the splice acceptor has the nucleotide sequence of SEQ ID NO:17; (3) a gene encoding a selectable marker, preferably a neomycin phosphotransferase expression cassette having the nucleotide sequence of SEQ ID NO:18; (4) a terminator, preferably the terminator has the nucleotide sequence of SEQ ID NO:19; and (5) an attB site having the nucleotide sequence of SEQ ID NO:8 or SEQ ID NO:9; and (c) a 3′ intron fragment, preferably the 3′ intron fragment has the nucleotide sequence of SEQ ID NO:15; (iii) a 3′ portion of the AAV rep gene, preferably the 3′ portion of the AAV rep gene has the nucleotide sequence of SEQ ID NO:56; (C) an AAV cap gene, preferably the AAV cap gene comprises the nucleotide sequence of SEQ ID NO:57; (D) a transgene flanked by a pair of AAV ITRs, preferably, the AAV ITR has the nucleotide sequence of SEQ ID NO:20, and the transgene comprises a promoter operably linked to a coding sequence, and the coding sequence is operably linked a polyadenylation signal; more preferably, the promoter has the nucleotide sequence of SEQ ID NO:21 and the polyadenylation signal has the nucleotide sequence SEQ ID NO:23; and (E) a second insulator, preferably the second insulator has the nucleotide sequence of SEQ ID NO:25.

In one aspect, provided herein is a cell comprising a non-naturally occurring nucleic acid molecule comprising, in 5′ to 3′ order: (A) a first insulator, preferably the first insulator has the nucleotide sequence of SEQ ID NO:24; (B) a modified AAV rep gene comprising, in 5′ to 3′ order: (i) a 5′ portion of an AAV rep gene, preferably the 5′ portion of the AAV rep gene has the nucleotide sequence of SEQ ID NO:73; (ii) an artificial intron comprising, in 5′ to 3′ order: (a) a 5′ intron fragment, preferably the 5′ intron fragment has the nucleotide sequence of SEQ ID NO:14; (b) a stop cassette comprising, in 5′ to 3′ order: (1) an attP site having the nucleotide sequence of SEQ ID NO:7; (2) a splice acceptor, preferably the splice acceptor has the nucleotide sequence of SEQ ID NO:17; (3) a gene encoding a selectable marker, preferably a neomycin phosphotransferase expression cassette having the nucleotide sequence of SEQ ID NO:18; (4) a terminator, preferably the terminator has the nucleotide sequence of SEQ ID NO:19; and (5) an attB site having the nucleotide sequence of SEQ ID NO:8 or SEQ ID NO:9; and (c) a 3′ intron fragment, preferably the 3′ intron fragment has the nucleotide sequence of SEQ ID NO:66; (iii) a 3′ portion of the AAV rep gene, preferably the 3′ portion of the AAV rep gene has the nucleotide sequence of SEQ ID NO:56; (C) an AAV cap gene; and (D) a transgene flanked by (i) a pair of AAV ITRs, preferably, the AAV ITR has the nucleotide sequence of SEQ ID NO:20, and the transgene comprises a promoter operably linked to a coding sequence, and the coding sequence is operably linked a polyadenylation signal; more preferably, the promoter has the nucleotide sequence of SEQ ID NO:21 and the polyadenylation signal has the nucleotide sequence SEQ ID NO:23; and (ii) a pair of spacer sequences, preferably, the spacer sequences have a nucleotide sequence of SEQ ID NO:67 and SEQ ID NO:68.

In one embodiment, the non-naturally occurring nucleic acid molecule is episomal, having the nucleotide sequence of SEQ ID NO:12. In another embodiment, the non-naturally occurring nucleic acid molecule is episomal, having the nucleotide sequence of SEQ ID NO:70.

In one embodiment, the cell further includes a nucleic acid molecule encoding a recombinase having the amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO:2; preferably, the nucleic acid comprises the nucleotide sequence at least 85%, at least 85%, 86%, 87%, 88%, 89%, 90%, 91%, 92%, 93%, 94%, 95%, 96%, 97%, 98%, 99%, or 100% identical to the nucleotide sequence of SEQ ID NO:3; more preferably, the cell comprises a recombinant ΔE1/ΔE3 adenovirus serotype 5 (Ad5) virus encoding the recombinase having the amino acid sequence of SEQ ID NO:2.

In one embodiment, the cell further includes adenovirus E1A and E1B genes, preferably the cell is a 911 cell, a pTG6559 cell, a GH329 cell, a N52.E6 cell, a HeLa-E1 cell, an UR cell, a VLI-293 cell, a HEK293 cell, or a PER.C6 cell.

In one aspect, provided herein is a method of producing a recombinant AAV comprising a transgene, comprising: (A) obtaining a first host cell comprising: (i) a modified AAV rep gene comprising, in 5′ to 3′ order: (a) a 5′ portion of an AAV rep gene, preferably the AAV rep gene has the nucleotide sequence of SEQ ID NO:55; (b) an artificial intron comprising, in 5′ to 3′ order: (1) a 5′ intron fragment, preferably the 5′ intron fragment has the nucleotide sequence of SEQ ID NO:14; (2) a stop cassette comprising, in 5′ to 3′ order: (aa) an attP site having the nucleotide sequence of SEQ ID NO:7; (bb) a splice acceptor, preferably the splice acceptor has the nucleotide sequence of SEQ ID NO:17; (cc) a gene encoding a selectable marker, preferably a neomycin phosphotransferase expression cassette having the nucleotide sequence of SEQ ID NO:18; (dd) a terminator, preferably the terminator has the nucleotide sequence of SEQ ID NO:19; and (ee) an attB site having the nucleotide sequence of SEQ ID NO:8 or SEQ ID NO:9; and (3) a 3′ intron fragment, preferably the 3′ intron fragment has the nucleotide sequence of SEQ ID NO:15; (c) a 3′ portion of the AAV rep gene, preferably the 3′ portion of the AAV rep gene has the nucleotide sequence of SEQ ID NO:56; (ii) an AAV cap gene, preferably the AAV cap gene comprises the nucleotide sequence of SEQ ID NO:57; and (iii) the transgene flanked by a pair of AAV ITRs, preferably, the ITR has the nucleotide sequence of SEQ ID NO:20, the transgene comprises a promoter operably linked to a coding sequence, and the coding sequence is operably linked a polyadenylation signal; more preferably, the promoter has the nucleotide sequence of SEQ ID NO:21 and the polyadenylation signal has the nucleotide sequence SEQ ID NO:23; (B) infecting the first host cell with a recombinant adenovirus comprising a recombinase gene encoding a recombinase having at least 85%, 86%, 87%, 88%, 89%, 90%, 91%, 92%, 93%, 94%, 95%, 96%, 97%, 98%, 99%, or 100%, sequence identity to the amino acid sequence of SEQ ID NO:2 to obtain a second host cell further containing the recombinase gene; (C) growing the second host cell under conditions in which the recombinant AAV comprising the transgene is produced; and (D) optionally collecting the recombinant AAV.

In one aspect, provided herein is a method of producing a recombinant AAV comprising a transgene, comprising: (A) obtaining a first host cell comprising: (i) a modified AAV rep gene comprising, in 5′ to 3′ order: (a) a 5′ portion of an AAV rep gene, preferably the AAV rep gene has the nucleotide sequence of SEQ ID NO:73; (b) an artificial intron comprising, in 5′ to 3′ order: (1) a 5′ intron fragment, preferably the 5′ intron fragment has the nucleotide sequence of SEQ ID NO:14; (2) a stop cassette comprising, in 5′ to 3′ order: (aa) an attP site having the nucleotide sequence of SEQ ID NO:7; (bb) a splice acceptor, preferably the splice acceptor has the nucleotide sequence of SEQ ID NO:17; (cc) a gene encoding a selectable marker, preferably a neomycin phosphotransferase expression cassette having the nucleotide sequence of SEQ ID NO:18; (dd) a terminator, preferably the terminator has the nucleotide sequence of SEQ ID NO:19; and (ee) an attB site having the nucleotide sequence of SEQ ID NO:8 or SEQ ID NO:9; and (3) a 3′ intron fragment, preferably the 3′ intron fragment has the nucleotide sequence of SEQ ID NO:66; (c) a 3′ portion of the AAV rep gene, preferably the 3′ portion of the AAV rep gene has the nucleotide sequence of SEQ ID NO:66; (ii) an AAV cap gene; and (iii) the transgene flanked by: (a) a pair of AAV ITRs, preferably, the ITR has the nucleotide sequence of SEQ ID NO:20, the transgene comprises a promoter operably linked to a coding sequence, and the coding sequence is operably linked a polyadenylation signal; more preferably, the promoter has the nucleotide sequence of SEQ ID NO:21 and the polyadenylation signal has the nucleotide sequence SEQ ID NO:23; and (b) a pair of spacer sequences, preferably, the spacer sequences have a nucleotide sequence of SEQ ID NO:67 and SEQ ID NO:68; (B) infecting the first host cell with a recombinant adenovirus comprising a recombinase gene encoding a recombinase having at least 85%, 86%, 87%, 88%, 89%, 90%, 91%, 92%, 93%, 94%, 95%, 96%, 97%, 98%, 99%, or 100%, sequence identity to the amino acid sequence of SEQ ID NO:2 to obtain a second host cell further containing the recombinase gene; (C) growing the second host cell under conditions in which the recombinant AAV comprising the transgene is produced; and (D) optionally collecting the recombinant AAV.

In one embodiment, the first host cell further comprises a first insulator upstream of the modified AAV rep gene and optionally a second insulator downstream of the transgene flanked by the ITRs, preferably, the first insulator and the second insulator are independently selected from the group consisting of: (a) a human anti-repressor element 40 having the nucleotide sequence of SEQ ID NO:24; (b) a mouse anti-repressor element 40 having the nucleotide sequence of SEQ ID NO:25; (c) an anti-repressor element 04 having the nucleotide sequence of GenBank accession number AY190749.1; (d) an anti-repressor element 06 having the nucleotide sequence of GenBank accession number AY190750.1; (e) an anti-repressor element 07 having the nucleotide sequence of GenBank accession number AY190751.1; (f) an anti-repressor element 12 having the nucleotide sequence of GenBank accession number AY190752.1; (g) an anti-repressor element 13 having the nucleotide sequence of GenBank accession number AY190753.1; (h) an anti-repressor element 35 having the nucleotide sequence of GenBank accession number AY190754.1; (i) an anti-repressor element 36 having the nucleotide sequence of GenBank accession number AY190755.1; (j) an anti-repressor element 52 having the nucleotide sequence of GenBank accession number AY190757.1; (k) an anti-repressor element 53 having the nucleotide sequence of GenBank accession number AY190758.1; and (l) a Chicken HS4 insulator from the globin locus having the nucleotide sequence of AY040835.1 in two or more copies, more preferably, the first insulator and the second insulator have the nucleotide sequences of SEQ ID NO:24 and SEQ ID NO:25, respectively.

In one embodiment, the first host cell comprises the first insulator upstream of the modified AAV rep gene, and further comprises a first spacer sequence and a second spacer sequence upstream and downstream of the transgene, respectively, wherein the first spacer sequence and the second spacer sequence are independently selected from the group consisting of: (a) a nucleotide sequence of SEQ ID NO:67; and (b) a nucleotide sequence of SEQ ID NO:68.

In one embodiment, the first host cell is obtained by introducing into a cell one or more nucleic acid molecules comprising the modified AAV rep gene, the AAV cap gene, the transgene flanked by the ITRs, the first insulator and the second insulator. In one embodiment, the first host cell is obtained by introducing into the cell a nucleic acid molecule comprising, in 5′ to 3′ order, the first insulator, the modified AAV rep gene, the AAV cap gene, the transgene flanked by the ITRs, the first insulator and the second insulator, preferably, a plasmid comprising the nucleotide sequence of SEQ ID NO:12.

In one embodiment, the first host cell is obtained by introducing into a cell one or more nucleic acid molecules comprising the modified AAV rep gene, the AAV cap gene, the transgene flanked by the ITRs, the first insulator, the first spacer sequence, and the second spacer sequence. In one embodiment, the first host cell is obtained by introducing into a cell one or more nucleic acid molecules comprising the modified AAV rep gene, the AAV cap gene, the transgene flanked by the ITRs, the first insulator, the first spacer sequence, and the second spacer sequencer, preferably, a plasmid comprising the nucleotide sequence of SEQ ID NO:70.

In one embodiment, the recombinant adenovirus is a recombinant ΔE1/ΔE3 adenovirus serotype 5 (Ad5) virus comprising a nucleotide sequence of SEQ ID NO:3.

In one embodiment, the host cell comprises adenovirus E1A and E1B genes, preferably the host cell is a 911 cell, pTG6559 cell, GH329 cell, N52.E6 cell, HeLa-E1 cell, UR cell, VLI-293 cell, HEK293 cell, or a PER.C6 cell.

In one embodiment, the conditions for growing the second host cell comprise culturing the second cell with 2-aminopurine. In one embodiment, the 2-aminopurine concentration is less than about 1.25 mM. In one embodiment, the 2-aminopurine concentration is about 1 μM to about 1.25 mM. In one embodiment, the 2-aminopurine concentration is about 10 μM to about 1.25 mM. In one embodiment, the 2-aminopurine concentration is about 100 μM to about 1.25 mM. In one embodiment, the 2-aminopurine concentration is about 1.25 mM.

In one embodiment, culturing the second cell with 2-aminopurine is initiated about 24 hours post-infection with the first host cell with a recombinant adenovirus.

In one aspect, provided herein is a composition comprising the cell comprising a nucleic acid molecule encoding a recombinase, as described above, and 2-aminopurine. In one embodiment, the 2-aminopurine concentration is less than about 1.25 mM. In one embodiment, the 2-aminopurine concentration is about 1 μM to about 1.25 mM. In one embodiment, the 2-aminopurine concentration is about 10 μM to about 1.25 mM. In one embodiment, the 2-aminopurine concentration is about 100 μM to about 1.25 mM. In one embodiment, the 2-aminopurine concentration is about 1.25 mM.

In one aspect, provided herein is a non-naturally occurring nucleic acid molecule comprising a nucleotide sequence encoding a serine recombinase having an amino acid sequence having at least 85%, 86%, 87%, 88%, 89%, 90%, 91%, 92%, 93%, 94%, 95%, 96%, 97%, 98%, 99% or 100%, identity to the amino acid sequence of SEQ ID NO:2. In one embodiment, the non-naturally occurring nucleic acid molecule includes a nucleotide sequence having at least 85%, 86%, 87%, 88%, 89%, 90%, 91%, 92%, 93%, 94%, 95%, 96%, 97%, 98%, 99% or 100%, identity to the nucleotide sequence of SEQ ID NO:3.

In one aspect, provided herein is a vector comprising the non-naturally occurring nucleic molecule comprising a nucleotide sequence encoding a serine recombinase having an amino acid sequence having at least 85%, 86%, 87%, 88%, 89%, 90%, 91%, 92%, 93%, 94%, 95%, 96%, 97%, 98%, 99% or 100%, identity to the amino acid sequence of SEQ ID NO:2.

In one aspect, provided herein is a vector comprising the non-naturally occurring nucleic molecule comprising a nucleotide sequence encoding a serine recombinase having an amino acid sequence having at least 85%, 86%, 87%, 88%, 89%, 90%, 91%, 92%, 93%, 94%, 95%, 96%, 97%, 98%, 99% or 100%, identity to the amino acid sequence of SEQ ID NO:3.

In one embodiment, the vector further includes a promoter, preferably a cytomegalovirus (CMV) promoter operably linked to the nucleotide sequence encoding the serine recombinase.

In one embodiment, the vector further includes a polyadenylation signal, such as a simian virus 40 (SV40) polyadenylation signal, operably linked to the nucleotide sequence encoding the serine recombinase.

In one embodiment, the vector is a DNA plasmid. In one embodiment, the vector is a recombinant adenoviral vector.

In one embodiment, the vector is a recombinant ΔE1/ΔE3 adenovirus serotype 5 (Ad5) virus comprising a nucleotide sequence encoding a serine recombinase having the amino acid sequence of SEQ ID NO:2 under the control of a CMV promoter, wherein the nucleotide sequence is further operably linked to a SV40 polyadenylation signal (NC_001669.1, nt 2550 to 2774).

In one aspect, provided herein is a cell comprising a non-naturally occurring nucleic acid molecule comprising a nucleotide sequence encoding a serine recombinase having an amino acid sequence having at least 85%, such as at least 85%, 86%, 87%, 88%, 89%, 90%, 91%, 92%, 93%, 94%, 95%, 96%, 97%, 98%, 99% or 100%, identity to the amino acid sequence of SEQ ID NO:2. In one embodiment, the cell includes a nucleotide sequence having at least 85%, such as at least 85%, 86%, 87%, 88%, 89%, 90%, 91%, 92%, 93%, 94%, 95%, 96%, 97%, 98%, 99% or 100%, identity to the nucleotide sequence of SEQ ID NO:3.

In one aspect, provided herein is a cell that includes the vector comprising the non-naturally occurring nucleic acid molecule comprising a nucleotide sequence encoding a serine recombinase having an amino acid sequence having at least 85%, such as at least 85%, 86%, 87%, 88%, 89%, 90%, 91%, 92%, 93%, 94%, 95%, 96%, 97%, 98%, 99% or 100%, identity to the amino acid sequence of SEQ ID NO:2. In another aspect, provided herein is a cell that includes the vector comprising the non-naturally occurring nucleic acid molecule comprising a nucleotide sequence encoding a serine recombinase having an amino acid sequence having at least 85%, such as at least 85%, 86%, 87%, 88%, 89%, 90%, 91%, 92%, 93%, 94%, 95%, 96%, 97%, 98%, 99% or 100%, identity to the amino acid sequence of SEQ ID NO:3.

In one embodiment, the cell further includes a promoter, preferably a cytomegalovirus (CMV) promoter operably linked to the nucleotide sequence encoding the serine recombinase.

In one embodiment, the cell further includes a polyadenylation signal, such as a simian virus 40 (SV40) polyadenylation signal, operably linked to the nucleotide sequence encoding the serine recombinase.

In one embodiment, the vector is a DNA plasmid. In one embodiment, the vector is a recombinant adenoviral vector.

In one embodiment, the recombinant adenoviral vector includes a recombinant ΔE1/ΔE3 adenovirus serotype 5 (Ad5) virus comprising a nucleotide sequence encoding a serine recombinase having the amino acid sequence of SEQ ID NO:2 under the control of a CMV promoter, wherein the nucleotide sequence is further operably linked to a SV40 polyadenylation signal (NC_001669.1, nt 2550 to 2774).

In one embodiment, the cell includes adenovirus E1A and E1B genes, preferably the cell is a 911 cell, pTG6559 cell, GH329 cell, N52.E6 cell, HeLa-E1 cell, UR cell, VLI-293 cell, HEK293 cell, or a PER.C6 cell.

In one aspect, provided herein is a method of conducting a site-specific recombination in a cell, comprising: (a) obtaining a cell comprising a nucleic acid molecule having an attP site having a nucleotide sequence at least 90%, 91%, 92%, 93%, 94%, 95%, 96%, 97%, 98%, 99% or 100%, identical to SEQ ID NO:7, preferably, an attP site having the nucleotide sequence of SEQ ID NO:7, and an attB site having a nucleotide sequence at least 90%, 91%, 92%, 93%, 94%, 95%, 96%, 97%, 98%, 99% or 100%, identical of SEQ ID NO:8 or SEQ ID NO:9, preferably, an attB site having the nucleotide sequence of SEQ ID NO:8 or SEQ ID NO:9; (b) introducing to the cell a non-naturally occurring nucleic acid molecule encoding a serine recombinase having at least 85%, 86%, 87%, 88%, 89%, 90%, 91%, 92%, 93%, 94%, 95%, 96%, 97%, 98%, 99% or 100% identity, to SEQ ID NO:2; and (c) growing the cell under conditions to allow the serine recombinase to catalyze the site-specific recombination between the attP and attB sites.

In one aspect, provided herein is a product produced by the process of conducting a site-specific recombination in a cell, comprising: (a) obtaining a cell comprising a nucleic acid molecule having an attP site having a nucleotide sequence at least 90%, 91%, 92%, 93%, 94%, 95%, 96%, 97%, 98%, 99% or 100%, identical to SEQ ID NO:7, preferably, an attP site having the nucleotide sequence of SEQ ID NO:7, and an attB site having a nucleotide sequence at least 90%, 91%, 92%, 93%, 94%, 95%, 96%, 97%, 98%, 99% or 100%, identical of SEQ ID NO:8 or SEQ ID NO:9, preferably, an attB site having the nucleotide sequence of SEQ ID NO:8 or SEQ ID NO:9; (b) introducing to the cell a non-naturally occurring nucleic acid molecule encoding a serine recombinase having at least 85%, 86%, 87%, 88%, 89%, 90%, 91%, 92%, 93%, 94%, 95%, 96%, 97%, 98%, 99% or 100% identity, to SEQ ID NO:2; and (c) growing the cell under conditions to allow the serine recombinase to catalyze the site-specific recombination between the attP and attB sites.

In one aspect, provided herein is a process for obtaining a product from a cell, comprising: (a) obtaining a cell comprising a nucleic acid molecule having an attP site having a nucleotide sequence at least 90%, 91%, 92%, 93%, 94%, 95%, 96%, 97%, 98%, 99% or 100%, identical to SEQ ID NO:7, preferably, an attP site having the nucleotide sequence of SEQ ID NO:7, and an attB site having a nucleotide sequence at least 90%, 91%, 92%, 93%, 94%, 95%, 96%, 97%, 98%, 99% or 100%, identical of SEQ ID NO:8 or SEQ ID NO:9, preferably, an attB site having the nucleotide sequence of SEQ ID NO:8 or SEQ ID NO:9; (b) introducing to the cell a non-naturally occurring nucleic acid molecule encoding a serine recombinase having at least 85%, 86%, 87%, 88%, 89%, 90%, 91%, 92%, 93%, 94%, 95%, 96%, 97%, 98%, 99% or 100% identity, to SEQ ID NO:2; (c) growing the cell under conditions to allow the serine recombinase to catalyze the site-specific recombination between the attP and attB sites; and (d) producing and recovering from the cell a product.

In one aspect, provided herein is a non-naturally occurring system, comprising: a means for AAV mediated recombination, wherein the means optionally comprises a transgenic element. In one aspect, provided herein is a means for transferring the non-naturally occurring system comprising: a means for AAV mediated recombination, wherein the means optionally comprises a transgenic element.

In one aspect, provided herein is a non-naturally occurring system, comprising: a recombination means for recombining the system of comprising: a means for AAV mediated recombination, wherein the means optionally comprises a transgenic element, wherein the recombination means includes using at least one serine residue during catalysis. In one aspect, provided herein is a means for transferring the non-naturally occurring system, comprising: a recombination means for recombining the system of comprising: a means for AAV mediated recombination, wherein the means optionally comprises a transgenic element, wherein the recombination means includes using at least one serine residue during catalysis.

In one aspect, provided herein is a means for manufacturing a molecule, wherein the means for manufacturing a molecule comprises the any of the means described above, and is capable of replication.

In one aspect, provided herein is a process for AAV mediated site-specific recombination, comprising: (a) a step for performing a function of obtaining a cell comprising a means for AAV mediated recombination, wherein the means optionally comprises a transgenic element; (b) a step for performing a function of growing the cell under conditions to allow site-specific recombination using at least one serine residue during catalysis. In one embodiment, the process includes obtaining a product, wherein, optionally the product is a therapeutic product.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows the alignment statistics and the sequence alignment of SPBetac2 integrase protein (SEQ ID NO:1, query) with a putative serine recombinase identified in the genome of Bacillus safensis strain CCMA-560 (SEQ ID NO:2, Sbjct), sequence ID: WP_029708089.1, with a length of 535 amino acids. The two proteins have 64% sequence identity at the protein level ranging from amino acids 1-529. This putative serine recombinase is named herein as SR21 (Serine Recombinase 21).

FIG. 2 shows the identification of a strain representing the pre-insertion locus: the alignment statistics and the sequence alignment of a CCMA-560 DNA sequence (query) with nucleotide 464352 to 464839 of whole genome shotgun sequence of Bacillus safensis strain Fairview contig56_1 (Sbjct), Sequence ID: NZ_JFBY01000018.1, with a length of 568093 nucleotides.

FIG. 3 shows SR21 recombinase attP and attB Sites. The attP and attB sites are composed of a dyad symmetry around a central dinucleotide recombination crossover site (underlined). Half sites are numbered. Spaces were introduced in attB sequences to show alignment of the sequence predicted to be bound by the zinc ribbon domain (ZD) and recombinase domain (RD) extrapolating from previous studies (Rutherford et al. (2013) Nucleic Acids Res. 41:8341-8356). Residues that are identical in three or four of the ZD or RD domains are in bold. The attP (SEQ ID NO:7) alignments to two alternate attB sequences (SEQ ID NO:8) and (SEQ ID NO:9) are shown.

FIG. 4 illustrates recombinase activation of reporter genes. Plasmid P41 encodes two reporter gene transcripts. The first driven by the EF1α promoter is constitutively active and encodes a fusion protein between green fluorescent protein (GFP) and renilla luciferase linked by a self-cleaving F2A peptide linker. The second transcript driven by CMV includes the SR21 Recombinase attB site (SEQ ID NO:9) followed by an inverted fusion protein coding region encoding mCherry and firefly luciferase linked by the P2A self-cleaving peptide linker and a SR21 attP site. Neither luciferase nor mCherry is expressed since they are in the opposite orientation relative the promoter. When SR21 recombinase is expressed, the attB and attP sequences are recombined, which results in the inversion of the reporter genes and expression of firefly luciferase and mCherry.

FIG. 5 shows AAV capsid proteins in purified recombinant AAV samples produced according to an embodiment of the application. Samples were purified from cells stably transfected with plasmid P439, grown and infected in Hyperflask vessels at 20 MOI (A) and 40 MOI (B) and were subjected to PAGE and silver staining.

FIG. 6 illustrates a rep/cap expression cassette with an artificial intron having a stop cassette inserted therein, according to an embodiment of the application.

FIG. 7 illustrates a vector (plasmid P439) according to an embodiment of the application.

FIG. 8 illustrates the position and sequence of RNA splice sites identified in P439 by RT-PCR. The top drawing represents the structure of the REP gene after STOP cassette excision. The 5′ and 3′ halves of REP are separated by the upstream half of the beta-Actin intron (SEQ ID NO:14), the SR21 AttL element (SEQ ID 35), and the downstream half of beta-Actin intron (SEQ ID NO:15). Splicing between (2) the beta-Actin splice donor (SEQ ID NO:71) and (3) beta-Actin splice acceptor (SEQ ID NO:72) are denoted by the solid line. Splicing between (1) an upstream splice donor in the 5′REP sequence (SEQ ID NO:64), and (3) the 3′ beta-Actin acceptor (SEQ ID NO:72) are shown with dotted lines. The sequences of splice donor and acceptors are shown below. Lower case sequence denotes the intron sequence.

FIG. 9 illustrates a vector (plasmid P600) according to an embodiment of the application.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the application can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.” Unless otherwise stated, any numerical value, such as a concentration or a concentration range described herein, are to be understood as being modified in all instances by the term “about.” Thus, a numerical value typically includes +10% of the recited value. For example, a concentration of 1 mg/mL includes 0.9 mg/mL to 1.1 mg/mL. Likewise, a concentration range of 1 mg/mL to 10 mg/mL includes 0.9 mg/mL to 11 mg/mL. As used herein, the use of a numerical range expressly includes all possible subranges, all individual numerical values within that range, including integers within such ranges and fractions of the values unless the context clearly indicates otherwise.

The phrases “percent (%) sequence identity” or “% identity” or “% identical to” when used with reference to an amino acid sequence describe the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 91%, 92%, 93%, 94%, 95%, 97%, 98%, 99%, or 100% identity over the full-length of the amino acid sequences) may be determined, when the sequences are compared and aligned for maximum correspondence as measured using a sequence comparison algorithm as known in the art, or when manually aligned and visually inspected. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g using the NCBI BLAST algorithm (Altschul S F, et al (1997), Nucleic Acids Res. 25:3389-3402).

As used herein, a “non-naturally occurring” nucleic acid or polypeptide, refers to a nucleic acid or polypeptide that does not occur in nature. A “non-naturally occurring” nucleic acid or polypeptide can be synthesized, treated, fabricated, and/or otherwise manipulated in a laboratory and/or manufacturing setting. In some cases, a non-naturally occurring nucleic acid or polypeptide can comprise a naturally-occurring nucleic acid or polypeptide that is treated, processed, or manipulated to exhibit properties that were not present in the naturally-occurring nucleic acid or polypeptide, prior to treatment. As used herein, a “non-naturally occurring” nucleic acid or polypeptide can be a nucleic acid or polypeptide isolated or separated from the natural source in which it was discovered, and it lacks covalent bonds to sequences with which it was associated in the natural source. A “non-naturally occurring” nucleic acid or polypeptide can be made recombinantly or via other methods, such as chemical synthesis.

As used herein, the term “hybrid” when used in reference to an AAV cap gene is intended to mean a cap gene that includes portions of one serotype capsid combined with portions of a different serotype capsid. The term also includes an AAV cap gene variant in which the naturally occurring AAV serotype sequence contains one or more non-naturally occurring mutations.

As used herein, the term “spacer sequence” is intended to mean a region of non-coding nucleotides that has no apparent function except to separation other genetic elements.

As used herein, the term “operably linked” refers to a linkage or a juxtaposition wherein the components so described are in a relationship permitting them to function in their intended manner. For example, a promoter is operably linked to a coding sequence if it affects the transcription of the coding sequence, or a signal sequence operably linked to an amino acid sequence of interest is capable of secret or translocate the amino acid sequence of interest over a membrane.

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples. For example, while embodiments of non-naturally occurring nucleic acids or recombinant vectors of the application (e.g., plasmid DNA or viral vectors) described herein may contain particular components, including, but not limited to, certain promoter sequences, enhancer or regulatory sequences, intron, coding sequence of AAV Rep and/or Cap, polyadenylation signal sequences, etc. arranged in a particular order, those having ordinary skill in the art will appreciate that the concepts disclosed herein may equally apply to other components arranged in other orders that can be used in nucleic acids or vectors of the application. The application contemplates use of any of the applicable components in any combination having any sequence that can be used in nucleic acids or vectors of the application, whether or not a particular combination is expressly described.

As used herein, a “vector” is a nucleic acid molecule used to carry genetic material into a cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). Preferably, a vector is a DNA plasmid. One of ordinary skill in the art can construct a vector of the application through standard recombinant techniques in view of the present disclosure.

A vector of the application can be an expression vector. As used herein, the term “expression vector” refers to any type of genetic construct comprising a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

In some embodiments of the application, a vector is a non-viral vector. Examples of non-viral vectors include, but are not limited to, DNA plasmids, bacterial artificial chromosomes, yeast artificial chromosomes, bacteriophages, etc. Preferably, a non-viral vector is a DNA plasmid. A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host cell. DNA plasmids used for expression of an encoded polynucleotide typically comprise an origin of replication, a multiple cloning site, and a selectable marker, which for example, can be an antibiotic resistance gene. Examples of DNA plasmids suitable that can be used include, but are not limited to, commercially available expression vectors for use in well-known expression systems (including both prokaryotic and eukaryotic systems), such as pSE420 (Invitrogen, San Diego, Calif.), which can be used for production and/or expression of protein in Escherichia coli; pYES2 (Invitrogen, Thermo Fisher Scientific), which can be used for production and/or expression in Saccharomyces cerevisiae strains of yeast; MAXBAC® complete baculovirus expression system (Thermo Fisher Scientific), which can be used for production and/or expression in insect cells; pcDNA™ or pcDNA3™ (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See, e.g., Sambrook et al., Molecular Cloning a Laboratory Manual, Second Ed. Cold Spring Harbor Press (1989)).

Preferably, a DNA plasmid is an expression vector suitable for protein expression in mammalian host cells. Expression vectors suitable for protein expression in mammalian host cells include, but are not limited to, pUC, pcDNA™, pcDNA3TM, pVAX, pVAX-1, ADVAX, NTC8454, etc. For example, the vector can be based on pUC57, containing a pUC origin of replication and ampicillin resistance gene (SEQ ID NO:30). It can further comprise a mammalian puromycin resistance gene cassette constructed from the Herpes virus thymidine kinase gene promoter (SEQ ID NO:26), the puromycin N-acetyl transferase coding region (SEQ ID NO:27), and a polyadenylation signal from bovine growth hormone gene (SEQ ID NO:28). The vector can also comprise an Epstein Barr Virus (EBV) OriP replication origin fragment (SEQ ID NO:29), which represents a composite of the ‘Dyad Symmetry’ region and the ‘Family of Repeats’ region of EBV.

A vector of the application can also be a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, lentiviral vectors, etc. The vector can also be a non-viral vector.

Preferably, a viral vector is an adenovirus vector, e.g., a recombinant adenovirus vector. As used herein, the terms “recombinant adenovirus vector” and “recombinant adenoviral vector” and “recombinant adenoviral particles” are used interchangeably and refer to a genetically-engineered adenovirus that is designed to insert a polynucleotide of interest into a eukaryotic cell, such that the polynucleotide is subsequently expressed. Examples of adenoviruses that can be used as a viral vector of the invention include those having, or derived from, the serotypes Ad2, Ad5, Ad11, Ad12, Ad24, Ad26, Ad34, Ad35, Ad40, Ad48, Ad49, Ad50, Ad52 (e.g., RhAd52), and Pan9 (also known as AdC68); these vectors can be derived from, for example, human, chimpanzee (e.g., ChAd1, ChAd3, ChAd7, ChAd8, ChAd21, ChAd22, ChAd23, ChAd24, ChAd25, ChAd26, ChAd27.1, ChAd28.1, ChAd29, ChAd30, ChAd31.1, ChAd32, ChAd33, ChAd34, ChAd35.1, ChAd36, ChAd37.2, ChAd39, ChAd40.1, ChAd41.1, ChAd42.1, ChAd43, ChAd44, ChAd45, ChAd46, ChAd48, ChAd49, ChAd49, ChAd50, ChAd67, or SA7P), or rhesus adenoviruses (e.g., rhAd51, rhAd52, or rhAd53). A recombinant adenovirus vector can for instance be derived from a human adenovirus (HAdV, or AdHu), or a simian adenovirus such as chimpanzee or gorilla adenovirus (ChAd, AdCh, or SAdV) or rhesus adenovirus (rhAd).

Preferably, an adenovirus vector is a recombinant human adenovirus vector, for instance a recombinant human adenovirus serotype 5, or any one of recombinant human adenovirus serotype 26, 4, 35, 7, 48, etc. A recombinant viral vector useful for the application can be prepared using methods known in the art in view of the present disclosure. For example, in view of the degeneracy of the genetic code, several nucleic acid sequences can be designed that encode the same polypeptide. A polynucleotide encoding a protein of interest can optionally be codon-optimized to ensure proper expression in the host cell (e.g., bacterial or mammalian cells). Codon-optimization is a technology widely applied in the art, and methods for obtaining codon-optimized polynucleotides will be well known to those skilled in the art in view of the present disclosure.

A non-naturally occurring nucleic acid molecule or a vector can comprise one or more expression cassettes. An “expression cassette” is part of a nucleic acid molecule or vector that directs the cellular machinery to make RNA and protein. An expression cassette can comprise a promoter sequence, an open reading frame, a 3′-untranslated region (UTR) optionally comprising a polyadenylation signal. An open reading frame (ORF) is a reading frame that contains a coding sequence of a protein of interest (e.g., Rep, Cap, recombinase or a recombinant protein of interest) from a start codon to a stop codon. Regulatory elements of the expression cassette can be operably linked to a polynucleotide sequence encoding a protein of interest.

A non-naturally occurring nucleic acid molecule or a vector of the application can contain a variety of regulatory sequences. As used herein, the term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e. mRNA) into the host cell or organism. Regulatory elements include, but are not limited to, a promoter, an enhancer, a polyadenylation signal, translation stop codon, a ribosome binding element, a transcription terminator, selection markers, origin of replication, etc.

A non-naturally occurring nucleic acid molecule or a vector can comprise a promoter sequence, preferably within an expression cassette, to control expression of a protein of interest. The term “promoter” is used in its conventional sense and refers to a nucleotide sequence that initiates the transcription of an operably linked nucleotide sequence. A promoter is located on the same strand near the nucleotide sequence it transcribes. Promoters can be a constitutive, inducible, or repressible. Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). For example, if the vector to be employed is a DNA plasmid, the promoter can be endogenous to the plasmid (homologous) or derived from other sources (heterologous). Preferably, the promoter is located upstream of the polynucleotide encoding a protein of interest within an expression cassette.

Examples promoters that can be used include, but are not limited to, a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. A promoter can also be a promoter from a human gene such as human actin, human myosin, human hemoglobin, human muscle creatine, or human metallothionein. A promoter can also be a tissue specific promoter, such as a muscle or skin specific promoter, natural or synthetic. Preferably, a promoter is a strong eukaryotic promoter, such as a cytomegalovirus (CMV) promoter (nt −672 to +15), EF1-alpha promoter, herpes virus thymidine kinase gene promoter (SEQ ID NO:26), etc.

A non-naturally occurring nucleic acid molecule or a vector can comprise additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for a protein of interest (e.g., Rep, Cap, recombinase) within an expression cassette of the vector. Enhancer sequences are regulatory DNA sequences that, when bound by transcription factors, enhance the transcription of an associated gene. An enhancer sequence is preferably downstream of a promoter sequence and can be downstream or upstream of a coding sequence within an expression cassette of the vector.

Any polyadenylation signal known to those skilled in the art in view of the present disclosure can be used. For example, the polyadenylation signal can be a SV40 polyadenylation signal (e.g., SEQ ID NO:60), AAV2 polyadenylation signal (bp 4411-4466, NC_001401.2), a polyadenylation signal from the Herpes Simplex Virus Thymidine Kinase Gene (SEQ ID NO:23), LTR polyadenylation signal, bovine growth hormone (bGH) polyadenylation signal, human growth hormone (hGH) polyadenylation signal, or human β-globin polyadenylation signal. Preferably, a polyadenylation signal is a bovine growth hormone (bGH) polyadenylation signal (SEQ ID NO:28), the polyadenylation signal of AAV2 having nucleotide numbers 4411 to 4466 of the nucleotide sequence of GenBank accession number NC_001401.2, or a SV40 polyadenylation signal (SEQ ID NO:60).

Any enhancer sequence known to those skilled in the art in view of the present disclosure can be used. For example, an enhancer sequence can be human actin, human myosin, human hemoglobin, human muscle creatine, or a viral enhancer, such as one from CMV, HA, RSV, or EBV. Examples of particular enhancers include, but are not limited to, Woodchuck HBV Post-transcriptional regulatory element (WPRE), intron/exon sequence derived from human apolipoprotein A1 precursor (ApoAI), untranslated R-U5 domain of the human T-cell leukemia virus type 1 (HTLV-1) long terminal repeat (LTR), a splicing enhancer, a synthetic rabbit (3-globin intron, or any combination thereof.

Preferably, an enhancer sequence comprises a P5 promoter of an AAV. The P5 promoter is part of a cis-acting Rep-dependent element (CARE) inside the coding sequence of the rep gene. CARE was shown to augment the replication and encapsidation when present in cis. CARE is also important for amplification of chromosomally integrated rep genes (if AAV ITRs are not present) as in some AAV producer cell lines. While not wishing to be bound by theories, it is believed that a P5 promoter placed downstream of a cap coding sequence potentially act as an enhancer to increase Cap expression, thus AAV yields, and that it also provides enhancer activity for amplifying genes integrated into a chromosome.

A non-naturally occurring nucleic acid molecule or a vector, such as a DNA plasmid, can also include a bacterial origin of replication and an antibiotic resistance expression cassette for selection and maintenance of the plasmid in bacterial cells, e.g., E. coli. An origin of replication (ORI) is a sequence at which replication is initiated, enabling a plasmid to reproduce and survive within cells. Examples of ORIs suitable for use in the application include, but are not limited to ColE1, pMB1, pUC, pSC101, R6K, and 15A, preferably pUC.

Vectors for selection and maintenance in bacterial cells typically include a promoter sequence operably linked to an antibiotic resistance gene. Preferably, the promoter sequence operably linked to an antibiotic resistance gene differs from the promoter sequence operably linked to a polynucleotide sequence encoding a protein of interest. The antibiotic resistance gene can be codon optimized, and the sequence composition of the antibiotic resistance gene is normally adjusted to bacterial, e.g., E. coli, codon usage. Any antibiotic resistance gene known to those skilled in the art in view of the present disclosure can be used, including, but not limited to, kanamycin resistance gene (Kan^{r}), ampicillin resistance gene (Amp^{r}), and tetracycline resistance gene (Tet^{r}), as well as genes conferring resistance to chloramphenicol, bleomycin, spectinomycin, carbenicillin, etc.

Vectors for selection and maintenance in mammalian cells typically include a promoter sequence operably linked to a gene encoding a protein that confers a selectable marker. Preferably, the gene further comprises a polyadenylation signal. For example, a mammalian puromycin resistance gene cassette can comprise a herpes virus thymidine kinase gene promoter (SEQ ID NO:26), a puromycin N-acetyl transferase coding region (SEQ ID NO:27), and a polyadenylation signal from bovine growth hormone gene (SEQ ID NO:28).

Manufacturing of recombinant AAV in human cells requires expression of AAV replication (rep) and capsid (cap) genes, adenovirus genes and an AAV-packageable transgene consisting of an expression cassette flanked by AAV inverted terminal repeats (ITRs). All three components can be delivered to cells on separate plasmids for AAV production, but existing transfection methods are difficult to scale to large-scale cultures. Incorporating some of these elements into the host cell line could make AAV production more efficient, however, some of the AAV and adenovirus genes are cytostatic or cytotoxic, limiting this approach.

The present application describes non-naturally occurring nucleic acid molecules, vectors, cells and methods to reversibly-inactivate the AAV rep gene such that AAV rep gene, AAV cap gene and a packageable transgene can be maintained and/or integrated into suitable host cells and expanded. Infection of these cells by a recombinant adenovirus expressing a recombinase reactivates the rep genes and induces AAV replication and packaging. Different from the approach described by Xiao and coworkers (Qiao et al. (2002) J. Virol. 76: 13015-13027; Yuan et al. (2011) Hum. Gene Ther. 22:613-624), which uses Cre, a tyrosine recombinase that recognizes two identical loxP sites and catalyzes both joining and excision reactions, the present invention uses Serine Recombinase 21 (SR21), a serine recombinase newly characterized by the inventors of this application. Unlike Cre, SR21 recognizes the attP and attB sites, which have different sequences. After the joining reaction catalyzed by SR21, the attP and attB sites are recombined and destroyed so that no additional recombination is possible. Thus, a method of the application can be more efficient than that catalyzed by Cre. Certain embodiments of the application include additional features, such as different stop cassette inserted in different artificial introns, enhancers, insulators, etc., which make further improvements to the approaches in the prior art. The reversible inactivation/reactivation system of the application allows the AAV rep gene to be tightly controlled during packaging cell growth to thus avoid the cytostatic/cytotoxic effect of the Rep proteins to the host cell. It also provides strong induction of the AAV rep gene and high yields of AAV vectors during production of the vectors.

### Serine Recombinase

Site specific recombination catalyzed by members of the large serine recombinase family (such as SR21) does not require cellular machinery for homologous recombination. Typically, it requires a specialized recombinase that recognizes the sites, breaks and joins the DNA. Based on amino acid sequence homology and mechanistic relatedness, most site-specific recombinases are grouped into one of two families: the tyrosine recombinase family or the serine recombinase family. The names stem from the conserved nucleophilic amino acid residue that they use to attack the DNA and which becomes covalently linked to it during strand exchange.

Serine recombinases bind and recombine separate recombination recognition sites known as “attachment sites”: attP, “attachment phage” and attB, “attachment bacterial” chromosome. The attP and attB sites are composed of a dyad symmetry around a central dinucleotide recombination crossover site. The left and right halves of attP and attB sites are bound by recombinase monomers by the zinc ribbon (ZD) and recombinase (RD) domains (Rutherford et al. (2013) Nucleic Acids Res. 41:8341-8356).

As described in more detail below in the Example, a serine recombinase, herein referred to as “Serine Recombinase 21” or “SR21” was newly identified in the present invention in the genome of Bacillus safensis strain CCMA-560. The attP and attB sites recognized by SR21 were also characterized in the present invention.

In one general aspect, the application relates to a non-naturally occurring nucleic acid molecule comprising a nucleotide sequence encoding a serine recombinase having an amino acid sequence having at least 85%, such as at least 85%, 86%, 87%, 88%, 89%, 90%, 91%, 92%, 93%, 94%, 95%, 96%, 97%, 98%, 99% or 100%, identity to the amino acid sequence of SEQ ID NO:2. Preferably, the non-naturally occurring nucleic acid molecule comprises a nucleotide sequence encoding a serine recombinase having the amino acid sequence of SEQ ID NO:2. In one embodiment, the non-naturally occurring nucleic acid molecule comprises a nucleotide sequence having at least 85%, such as at least 85%, 86%, 87%, 88%, 89%, 90%, 91%, 92%, 93%, 94%, 95%, 96%, 97%, 98%, 99% or 100%, identity to the nucleotide sequence of SEQ ID NO:3.

In certain embodiments, the application relates to a vector comprising the non-naturally occurring nucleic acid. The vector can be an expression vector that expresses the serine recombinase in a cell of interest, e.g., a bacterial cell or a mammalian cell. In one embodiment, the vector expresses the serine recombinase in a mammalian cell under control of a cytomegalovirus (CMV) promoter or any other suitable promoter described herein or known in the art. In certain embodiments, the vector can further include a polyadenylation signal, such as a simian virus 40 (SV40) polyadenylation signal or any other suitable polyadenylation signal described herein or known in the art.

In one embodiment, the vector is a DNA plasmid, such as plasmid P175 having the nucleotide sequence of SEQ ID NO:10.

In another embodiment, the vector is a viral vector, such as a recombinant adenoviral vector.

In one embodiment, the vector is a recombinant ΔE1/ΔE3 adenovirus serotype 5 (Ad5) virus comprising a nucleotide sequence encoding a serine recombinase having at least 85% identity, such as at least 85%, 86%, 87%, 88%, 89%, 90%, 91%, 92%, 93%, 94%, 95%, 96%, 97%, 98%, 99% or 100% identity, to the amino acid sequence of SEQ ID NO:2, and the coding sequence is under the control of a promoter functional in a mammalian cell. Preferably, the promoter is a CMV promoter. More preferably, the recombinant Ad5 vector comprises, in 5′ to 3′ order, a CMV promoter operably linked to a nucleotide sequence encoding the amino acid sequence of SEQ ID NO:2, which is operably linked to a SV40 polyadenylation signal (NC_001669.1, nt 2550 to 2774). In one embodiment, the nucleotide sequence encoding the amino acid sequence of SEQ ID NO:2 is the same as SEQ ID NO:3 except that the bacterial translation initiation codon “TTG” is replaced by an “ATG”, and three point-mutations were introduced to destroy restriction endonuclease recognition sites within SEQ ID NO:3. These restriction endonuclease recognition sites are Xba I site (TCTAGA); Sac I site (GAGCTC); EcoRI site (GAATTC).

A vector encoding a serine recombinase of the application can be made using any methods known in the art in view of the present disclosure.

As described in more detail in the Example below, attP and attB sites for a serine recombinase of the application are identified in the present invention. In certain embodiments, a serine recombinase of the application recognizes an attP site comprising the nucleotide sequence of SEQ ID NO:7 or a variant thereof. In certain embodiments, a serine recombinase of the application recognizes an attP site comprising a nucleotide sequence at least 90%, such as at least 91%, 92%, 93%, 94%, 95%, 96%, 97%, 98%, 99% or 100%, identical to SEQ ID NO:7.

In certain embodiments, a serine recombinase of the application recognizes an attB site comprising the nucleotide sequence of SEQ ID NO:8 or SEQ ID NO:9, or a variant thereof. In certain embodiments, a serine recombinase of the application recognizes an attB site comprising a nucleotide sequence at least 90%, such as at least 91%, 92%, 93%, 94%, 95%, 96%, 97%, 98%, 99% or 100%, identical to SEQ ID NO:8 or SEQ ID NO:9.

In one embodiment, the application relates to a method of conducting a site-specific recombination in a cell. The method comprises:
1) obtaining a cell comprising a nucleic acid molecule having an attP site having a nucleotide sequence at least 90% identical to SEQ ID NO:7, and an attB site having a nucleotide sequence at least 90% identical to SEQ ID NO:8 or SEQ ID NO:9;
2) introducing to the cell a non-naturally occurring nucleic acid molecule encoding a serine recombinase having at least 85% identity, such as at least 85%, 86%, 87%, 88%, 89%, 90%, 91%, 92%, 93%, 94%, 95%, 96%, 97%, 98%, 99% or 100% identity, to SEQ ID NO:2; and
3) growing the cell under conditions to allow the serine recombinase to catalyze the site-specific recombination between the attP and attB sites.

In a preferred embodiment, the application relates to a method of conducting a site-specific recombination in a cell. The method comprises:
1) obtaining a cell comprising a nucleic acid molecule having an attP site having the nucleotide sequence of SEQ ID NO:7, and an attB site having the nucleotide sequence of SEQ ID NO:8 or SEQ ID NO:9;
2) introducing to the cell a non-naturally occurring nucleic acid molecule encoding a serine recombinase having the amino acid sequence of SEQ ID NO:2; and
3) growing the cell under conditions to allow the serine recombinase to catalyze the site-specific recombination between the attP and attB sites.

### Constructs, Cells and Methods for Production of Recombinant AAV

As illustrated in the Example below, the newly identified serine recombinase of the application can be used to improve production of recombinant AAVs.

### Modified AAV Rep Gene Construct

In one general aspect, the application is related to a non-naturally occurring nucleic acid molecule comprising a modified adeno-associated virus (AAV) rep gene, which has an AAV rep gene encoding four Rep proteins Rep78, Rep68, Rep52 and Rep40 and an artificial intron inserted into a coding sequence of the rep gene shared by the four Rep proteins. The artificial intron comprises a stop cassette inserted downstream of the 5′ splice site and upstream of the branch site of the artificial intron, and the stop cassette comprises, in 5′ to 3′ order: (i) an attP site having a nucleotide sequence at least 90%, such as at least 91%, 92%, 93%, 94%, 95%, 96%, 97%, 98%, 99% or 100%, identical to SEQ ID NO:7, (ii) a splice acceptor; (iii) a terminator; and (iv) an attB site having the nucleotide sequence at least 90%, such as at least 91%, 92%, 93%, 94%, 95%, 96%, 97%, 98%, 99% or 100%, identical to SEQ ID NO:8 or SEQ ID NO:9. Preferably, the attP site has the nucleotide sequence of SEQ ID NO:7 and the attB site has the nucleotide sequence of SEQ ID NO:8 or SEQ ID NO:9.

As used herein, an “intron” is broadly defined as a sequence of nucleotides that is removable by RNA splicing. “RNA splicing” means the excision of introns from a pre-mRNA to form a mature mRNA. An “artificial intron” as used herein refers to a sequence of nucleotides that is not a naturally occurring intron for a gene but is nonetheless removable by RNA splicing. For example, an “artificial intron” can be a naturally occurring intron with an inserted stop cassette.

An intron, including an artificial intron, contains a 5′ splice site or junction, a splice acceptor or branch point, and a 3′ splice site or splice junction. The term “5′ splice site” or “5′ splice junction” means the location of the exon-intron junction wherein the junction is between the 3′ end of the 5′ fragment of a gene or nucleic acid fragment and the 5′ end of the intron, and includes the consensus sequence at the 5′ end of the intron that is required for RNA splicing. The term “splice acceptor” or “branch point” refers to the nucleotide, usually adenosine, located approximately 20-50 bp from the 3′ splice site that helps form the lariat structure during the first trans-esterification reaction during RNA splicing. The term “3′ splice site” or “3′ splice junction” means the location of the exon-intron junction wherein the junction is between the 5′ end of the 3′ fragment of a gene or nucleic acid fragment and the 3′ end of the intron, and also includes the consensus sequence at the 3′ end of the intron that is required for RNA splicing. The term “consensus sequence” means the nucleotides in/or adjacent to either the 5′ or 3′ splice junction that are required for RNA splicing; these sequences are usually either invariant or highly conserved.

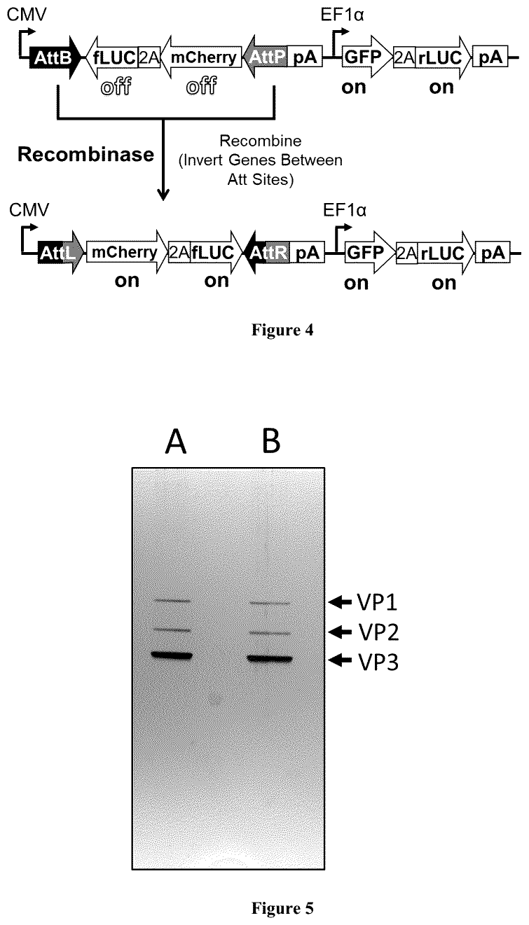

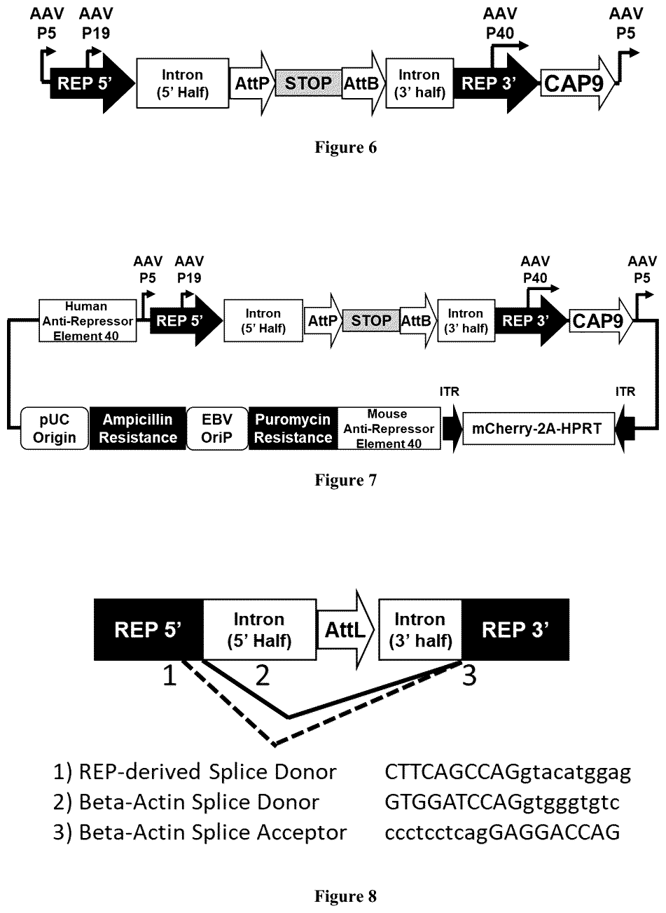

Analysis of a large number of mRNAs has revealed that certain nucleotides are conserved in typical introns and splice junctions. For example, nearly invariant bases of an intron are the 5′-GU and the 3′-AG. Certain bases that flank these 5′ and 3′ conserved regions often are found in abnormal (non-random) frequencies. Also conserved is the branch-point adenosine, usually 20-50 bases from the 3′ splice site. See, e.g., FIG. 4 of Gao et al (2008) Nucleic Acids Research 36: 2257-2267, that shows the general consensus for introns in the context of an exon, the entire content of Gao et al (2008) is incorporated herein by reference. However, the central region of the intron, which may range from 40 to 50,000 bases in length, is generally unnecessary for splicing to occur. Introns are removed from RNA or pre-mRNA as a lariat structure by spliceosomes. The splicing together of exons proceeds via two sequential transesterification reactions.

Insertion of an intron into an expressed sequence can be accomplished by any method known in the art. The flanking exonic context as well the actual intronic sequence to be used play a role in whether the new “intron” will be effectively spliced out. Introns suitable for the invention can be tested by making composite sequences in silico and using online splice prediction programs to find combinations of the rep gene sequence and intron sequences that give high enough scores for efficient RNA splicing. Any of the introns in the genome or synthetic sequences can be tested and optimized for use in constructs of the invention in view of the present disclosure.

To disrupt expression of all four rep open reading frames for Rep78, Rep68, Rep52 and Rep40, an artificial intron is preferably inserted into a coding sequence of the rep gene shared by the four Rep proteins. Accordingly, in certain embodiments, to disrupt all four ORFs, an artificial intron is inserted after nucleotides 996 and up to 1905 of AAV2 (NC_001401.2) or corresponding positions in another AAV rep gene. But for the stop cassette to work when inserted in the artificial intron, it is preferred to have the intron inserted in the rep gene as far upstream as possible.

Additionally, the exonic context just upstream and downstream of the intron insertion site is important to defining what will work as a possible insertion site, e.g., the general consensus for introns in the context of an exon discussed above. In one embodiment, the consensus sequence CAG\hat{ }G (where \hat{ } marks where the insertion would go) occurs in the relevant region of rep gene in AAV2 as follows where the number indicates the last nucleotide of AAV before the insertion: 1052, 1061, 1712, and 1906. In another embodiment, the consensus sequence AAG\hat{ }G occurs in locations 1022 (as used by Qiao), 1112, 1475, 1514, 1700, 1742, and 1784 of AAV2. Other consensus sites, such as AAG\hat{ }A, occur at, e.g., nucleotide 1340 of AAV2. The preferred insertion site can also be identified in rep genes of other AAVs in view of the present disclosure.

The artificial intron useful for the invention can be derived from any source, such as from a genomic library. An intron can be obtained by polymerase chain reaction (PCR) from human DNA using primers, as described below. Any intron capable of RNA splicing in cells can be used in the method of the present invention. In the Example below, the intron is an intron of human R-Actin gene.

According to embodiments of the application, in addition to RNA splicing via an artificial intron, the expression of Rep proteins is also regulated by DNA splicing via a stop cassette inserted into the artificial intron. The stop cassette comprises a transcription terminator flanked by the attP and attB sites specifically recognized by a serine recombinase, such as that characterized in the invention. In one embodiment, the terminator comprises one or more polyadenylation signals. In another embodiment, the terminator comprises another sequence for efficient transcription termination, such as a sequence from the human β-globin gene downstream of the polyadenylation signal that encodes a self-cleaving RNA motif, preferably having the nucleotide sequence of SEQ ID NO:19. Other terminators can also be used in the invention, such as a hammerhead ribozyme that cleave its own RNA. See West (2008) Molecular Cell 29:600-610 for use of other ribozyme replacing the beta globin element, and Kharma (2016) Nucleic Acids Res. 44:e39 for description of designing ribozymes, the contents of both are incorporated herein by reference in their entireties.

In one embodiment, the stop cassette further comprises a gene encoding a selectable marker. In one embodiment, the selectable marker gene comprises a neomycin phosphotransferase expression cassette (neo) (SEQ ID NO:18), which is driven by a mammalian promoter (e.g., mouse phosphoglycerate kinase 1) and a bacterial (e.g., Lac zya) promoter and followed by a polyadenylation signal, such as that from SV40. This gene confers resistance to neomycin and kanamycin in mammalian and bacterial cells, respectively. While not wishing to be bound by theories, it is believed that, in addition to serving a selectable marker for cell line development, a selectable marker gene can further block the transcription of the rep gene to thereby increase the stability of a host cell containing the modified rep gene. Other selectable marker genes that can be used in the invention include, but are not limited to, antibiotic selection genes (puromycin, hygromycin, bleomycin), a metabolic gene (e.g. glutamine synthase or hypoxanthine-guanine phosphoribosyltransferase (HPRT)), a visual marker such as mCherry, an enzyme such as beta-glactosidase, secreted alkaline phosphatase, or any other suitable marker genes.

In another embodiment, the stop cassette comprises a splice acceptor to prevent the stop cassette from being splice out of primary mRNA transcripts. Any naturally occurring splice acceptor site or synthetic sequence can be used, provided that the splice acceptor is not skipped. According to embodiments of the application, the splice acceptor contains a branch point sequence conforming to the consensus (yTnAynn), wherein y is a C or T and n is any nucleotide, a polypyrimidine tract (4-24 nt), an “AG” dinucleotide and a eukaryotic gene exon sequence (or synthetic sequence that acts like an exon when placed next to the intron sequence) of 20-80 bp. The sequence should be recognized as a splice acceptor site by NetGene2 Splice prediction software (www.cbs.dtu.dk/services/NetGene2/; Brunak, S., Engelbrecht, J., and Knudsen, S.: Prediction of Human mRNA Donor and Acceptor Sites from the DNA Sequence, Journal of Molecular Biology, 1991, 220, 49-65) with a confidence score of 0.4 or better (or with similar splice prediction software); scores closer to 1.0 are better. In one embodiment, the splice acceptor comprises the nucleotide sequence of SEQ ID NO:17 (NC_000086.7, nucleotides 53001998 to 53002138 from the mouse HPRT gene, plus a 29 nt region from the human agouti signaling protein (NC_000020.11, nucleotides 34262765 to 34262793).

According to embodiments of the application, the stop cassette is inserted downstream of the 5′ splice donor site and upstream of the splice acceptor “branch point” of the artificial intron.

The stop cassette can be inserted at any position between the two sites, provided that the insertion does not damage the functions of the sites. In one embodiment, the stop cassette is inserted in the middle of the two sites. In one exemplary embodiment described in the Example below, the stop cassette is inserted into the intron of human 3-Actin gene such that the 5′ intron fragment has the nucleotide sequence of SEQ ID NO:14 and the 3′ intron fragment has the nucleotide sequence of SEQ ID NO:15.

As provided herein, in some embodiments the 3′ intron fragment can include a spacer sequence that makes the REP/CAP gene too large to package in AAV. For example, the AAV packaging limit is approximately 5.0 kb. Thus, a spacer sequence that makes the REP/CAP gene greater than approximately 5.0 kb can be generated according to the disclosure provided herein. In some embodiments, the spacer sequence is a 2 kb random spacer inserted in the 3′ intron fragment. Accordingly, in an exemplary embodiment described in the Example below, the stop cassette is inserted into the intron of human 3-Actin gene such that the 5′ intron fragment has the nucleotide sequence of SEQ ID NO:14 and the 3′ intron fragment has the nucleotide sequence of SEQ ID NO:66. However, it is understood that the spacer sequence need not be 2 kb, and can be any length that results in the REP/CAP gene being larger than approximately 5.0 kb.

Any AAV rep gene can be included in the modified rep gene of the invention. For example, the AAV rep gene can comprise a rep gene of one of AAV1 to AAV8, or a hybrid thereof. The sequences of the AAV rep gene are available from, e.g., GenBank, with the following GenBank accession numbers for the various AAV genomes: AAV1, GenBank accession No. NC_002077.1; AAV2, GenBank accession No. NC_001401.2; AAV3, GenBank accession No. NC_001729.1; AAV4, GenBank accession No. NC_001829.1; AAV5, GenBank accession No. NC_006152.1; AAV6, GenBank accession No. AF028704.1; AAV7, GenBank accession No. NC_006260.1; and AAV8, GenBank accession No. NC_006261.1.

In the Example below, a rep gene of human AAV2 having nucleotide numbers 190 to 2202 of the nucleotide sequence of GenBank accession number NC_001401.2 is used.

In some embodiments, modifications to a cryptic splice site in the rep gene can be made to eliminate splicing at this site. For example, a synonymous mutation to the DNA sequence can be made in which the DNA sequence is mutated, but the mutation does not change the encoded amino acid.

Constructs with a Modified AAV Rep Gene and an AAV Cap Gene

In another general aspect, the application relates to a non-naturally occurring nucleic acid molecule comprising a modified AAV rep gene of the application and an AAV cap gene, or hybrid thereof. Preferably, the AAV cap gene is downstream of the modified AAV rep gene.

In one embodiment, the AAV cap gene further comprises a polyadenylation signal operably linked to a coding sequence of the gene. In an exemplary embodiment described in the example below, an AAV2 polyadenylation signal (bp 4411-4466, NC_001401.2) is included downstream of the AAV9 cap coding sequence.

In another embodiment, the AAV cap gene further comprises an enhancer. In the example below, an AAV2 rep P5 promoter (bp 190-313, NC_001401.2) is included downstream of the AAV2 polyadenylation signal.

In certain embodiment, the AAV cap gene encodes all three of the capsid proteins VP1, VP2 and VP3.

In other embodiments, the AAV cap gene encodes less than three of the capsid proteins. For example, it was reported that AAV serotypes 1 through 5 could successfully package, replicate in, and transduce cells without VP2 (Grieger et al., J Virol. 2005 August; 79(15): 9933-9944). According, in one embodiment, the AAV cap gene encodes VP1 and VP3, but not VP2, of any of AAV 1 to AAV5, or a hybrid thereof.

Any AAV cap gene can be used in the invention. For example, the AAV cap gene can be a cap gene of one of AAV1 to AAV8, AAV9, AAVDJ, or a hybrid thereof. In one embodiment, the cap gene is an AAV9 variant. The sequences of the AAV cap gene are available from, e.g., GenBank. See the above described GenBank accession numbers for the AAV1 to AAV8 genomes. The AAV9 genome has the GenBank accession No. AY530579.1, and the AAVDJ has the GenBank protein accession No, 3J1Q_A.

In one embodiment, described in the Example below, a cap open reading frame of human AAV9 having the nucleotide sequence of GenBank accession number AY530579.1 is used.

Constructs with a Modified AAV Rep Gene, an AAV Cap Gene and a Transgene

In another general aspect, the application relates to a non-naturally occurring nucleic acid molecule comprising a modified AAV rep gene of the application, an AAV cap gene and a transgene flanked by AAV Inverted Terminal Repeats (ITRs).

The ITRs are important cis-active sequences in the biology of AAV. A key role of the ITRs is in AAV DNA replication. In addition to its role in AAV replication, the ITR is also essential for AAV genome packaging, transcription, negative regulation under nonpermissive conditions, and site-specific integration.

In one embodiment, a 130 bp ITR comprises the nucleotide sequence of SEQ ID NO:20 derived from the 3′ AAV2 ITR (Nucleotides 4535-4664, NC_001401.2) is used to flank the transgene. In another embodiment, a shorter mutated ITR is used. For example, for shorter genes, an ITR is mutated to be shorter and the gene can fold into a double-stranded form to increase expression and speed up expression after infection. See McCarty 2008 Mol Ther. 2008; 16(10):1648-56.

In another embodiment, the transgene comprises a promoter, preferably a promoter functional in a mammalian cell. In the Example described below, a human EF1-alpha promoter (including exon 1, intron 1, and part of exon 2) (SEQ ID NO:21) is included in the transgene.

In another embodiment, the transgene comprises a polyadenylation signal. In the Example described below, a polyadenylation signal from the Herpes Simplex Virus Thymidine Kinase Gene (SEQ ID NO:23) is included in the transgene.

In yet another embodiment, a non-naturally occurring nucleic acid molecule comprises a pair of insulators flanking a modified AAV rep gene, an AAV cap gene and an ITR flanked transgene. In another embodiment, a non-naturally occurring nucleic acid molecule comprises a single insulator upstream of a modified AAV rep gene, an AAV cap gene and an ITR flanked transgene. In one embodiment, the insulator comprises genomic elements that block chromatin-associated repression of gene expression (Kwaks et al. (2003) Nature Biotechnology 21: 554-558; Kwaks et al. (2003) Nature Biotechnology 21: 822).

Any suitable insulator, such as those described herein, can be used in the invention. In one embodiment the insulator is a human anti-repressor element 40 having the nucleotide sequence of SEQ ID NO:24. In another embodiment, the insulator is a mouse anti-repressor element 40 having the nucleotide sequence of SEQ ID NO:25. In another embodiment, In another embodiment, the insulator is an anti-repressor element 04 having the nucleotide sequence of GenBank accession number AY190749.1. In another embodiment, the insulator is an anti-repressor element 06 having the nucleotide sequence of GenBank accession number AY190750.1. In another embodiment, the insulator is an anti-repressor element 07 having the nucleotide sequence of GenBank accession number AY190751.1. In another embodiment, the insulator is an anti-repressor element 12 having the nucleotide sequence of GenBank accession number AY190752.1. In another embodiment, the insulator is an anti-repressor element 13 having the nucleotide sequence of GenBank accession number AY190753.1. In another embodiment, the insulator is an anti-repressor element 35 having the nucleotide sequence of GenBank accession number AY190754.1. In another embodiment, the insulator is an anti-repressor element 36 having the nucleotide sequence of GenBank accession number AY190755.1. In another embodiment, the insulator is an anti-repressor element 52 having the nucleotide sequence of GenBank accession number AY190757.1. In another embodiment, the insulator is an anti-repressor element 53 having the nucleotide sequence of GenBank accession number AY190758.1. In another embodiment, the insulator is a Chicken HS4 insulator from the globin locus having the nucleotide sequence of AY040835.1 in two or more copies.

The non-naturally occurring nucleic acid molecule that comprises a pair of insulators can have the same or different insulators as a pair to flank the gene segment of interest. Preferably, different insulators are used as a pair to flank the gene segment of interest. In one exemplary embodiment described the Example below, human anti-repressor element 40 (AY190756.1, SEQ ID NO:24) and mouse anti-repressor element 40 (SEQ ID NO:25) are used as the insulators. In another exemplary embodiment described the Example below, human anti-repressor element 40 (AY190756.1, SEQ ID NO:24) is used as the insulator.

As provided herein, the constructs on the present disclosure can also include spacer sequences on both sides of the AAV transgene to decrease the risk of mispackaging other vector components. In one embodiment, the non-naturally occurring nucleic acid molecule comprises a first and a second spacer sequence upstream and downstream of the transgene, respectively. In certain embodiments, the spacer sequences are 2 kb spacer sequences. In a specific embodiment, the non-naturally occurring nucleic acid molecule comprises the first insulator upstream of the modified AAV rep gene, and further comprises a first and a second spacer sequence upstream and downstream of the transgene, respectively, wherein the first insulator and the second spacer sequence are independently selected from the group consisting of: (a) a nucleotide sequence of SEQ ID NO:67; and (b) a nucleotide sequence of SEQ ID NO:68.

Cells and Methods for Production of Recombinant AAV Expression of the Rep proteins from a modified AAV rep gene of the application is under tight control by both DNA splicing and RNA splicing mechanisms, thus allowing stable host cells containing the modified rep gene to be generated and grown to high numbers in a bioreactor. For AAV production, a stable host cells containing a modified AAV rep gene, an AAV cap gene and a transgene flanked by ITRs are first grown to high numbers, then infected with a replication deficient adenovirus expressing a serine recombinase that recognizes the attP and attB sites in the modified AAV rep gene. A site-specific recombination between the attP and attB sites catalyzed by the serine recombinase splices out the stop cassette, resulting in the production of a pre-mRNA comprising both the 5′ and 3′ rep coding sequences separated by a functional intron. The intron is then excised by ubiquitous cellular machinery (spliceosomes), resulting in an mRNA encoding the four Rep proteins, allowing production of AVVs at high titer.

Stable host cells containing a modified AAV rep gene, an AAV cap gene and a transgene flanked by ITRs can be obtained by transducing a cell with one or more nucleic acid molecules encoding the genes. In one embodiment, a stable host cell is obtained by transducing a cell with a first nucleic acid molecule encoding a modified AAV rep gene and an AAV cap gene to obtain a first host cell comprising the modified AAV rep gene and AAV cap gene, and further transducing the first host cell with a second nucleic acid molecule encoding a transgene flanked by ITRs. In one embodiment, the modified AAV rep gene and the AAV cap gene are stably integrated into the chromosome of the first host cell. In another embodiment, the modified AAV rep gene and the AAV cap gene remain episomal in the first host cell. The transgene flanked by ITRs can also be stably integrated into the host cell or remain episomal.

In another embodiment, a stable host cell is obtained by transducing a cell with a nucleic acid molecule encoding a modified AAV rep gene, an AAV cap gene and a transgene flanked by ITRs. The modified AAV rep gene, the AAV cap gene and the transgene flanked by ITRs can be stably integrated into the host cell or remain episomal.

The stable host cells can be grown to high cell density before being infected with an adenovirus expressing a serine recombinase.

A replication deficient adenovirus expressing a serine recombinase of the application is introduced to the stable host cells using any methods known in the art in view of the present disclosure. In one embodiment, the replication deficient adenovirus is a recombinant ΔE1/ΔE3 adenovirus serotype 5 (Ad5) virus comprising a nucleotide sequence encoding an amino acid sequence at least 85% identical to the amino acid sequence of SEQ ID NO:2, preferably 100% identical to SEQ ID NO:2. For example, the adenovirus can comprise a nucleotide sequence that is at least 85% identical to SEQ ID NO:3, preferably at least 95% identical to SEQ ID NO:3.

As disclosed herein, the present disclosure also includes methods and compositions for increasing AAV production by contacting the cells described herein with 2 aminopurine (2-AP). At late stages of the adenovirus life cycle, the virus inhibits host protein synthesis. This results in part from the actions of the late adenovirus 100-kilodalton (kDa) protein, which displaces Mnk1 kinase from the cap-initiation complex eIF4F, leading to dephosphorylation of eIF4E and the inhibition of cap-dependent mRNA translation (see, e.g., Cuesta (2004), J. Virology 78: 7707-7716). Adenoviral late gene transcripts include a tripartite leader sequence at their 5′ end that promotes translation by a mechanism called ribosome shunting (see, e.g., Yueh (2000) Genes Dev 14: 414-421). In the context of an AAV producer cell line, inhibition of cap-dependent translation is predicted to block expression of AAV REP and CAP genes as well as early adenoviral proteins needed for AAV replication and packaging. Thus, in some embodiments, the cells are cultured with chemicals that block host protein translation shutdown to increase the efficiency of AAV producer cell lines using an adenoviral inducer.

In certain embodiments, the chemical that blocks host protein translation shutdown is 2-aminopurine (2-AP). 2-AP has been shown to block the shutdown of host protein synthesis induced by adenovirus (see, e.g., Zhang and Schneider (1994) J. Virology 68: 2544-2555; Huang and Schneider (1990) PNAS 87: 7115-7119). Treatment of AAV producing cells with 2-AP was able to reduce the cytopathic effects of infection including restoration of the cytokeratin network normally degraded by late infection (Zhang and Schneider (1994) J. Virology 68: 2544-2555). 2-AP inhibits a number of kinases in vitro including the RNA-dependent protein kinase PKR (also known as eukaryotic translation initiation factor 2 alpha kinase 2, EIF2AK2) (DeBenedetti (1983) J Biol Che, 258: 14556-14562), but was unable to block PKR activation in cells and the phosphorylation of eIF-2a that occurs after adenoviral infection (Huang and Schneider (1990) PNAS 87: 7115-7119). 2-AP did increase the early adenovirus DNA-binding protein (DBP) levels 10 to 20-fold without increasing mRNA levels (Huang and Schneider (1990) PNAS 87: 7115-7119), consistent with an effect on cap-dependent translation.

Accordingly, in some embodiments, the method of producing a recombinant AAV comprising a transgene includes culturing cells of the present disclosure with 2-aminopurine. In some embodiments, the 2-aminopurine concentration is less than about 10 mM. In some embodiments, the 2-aminopurine concentration is less than about 5 mM. In some embodiments, the 2-aminopurine concentration is less than about 2.25 mM. In some embodiments, the 2-aminopurine concentration is less than about 1.25 mM. In some embodiments, the 2-aminopurine concentration is about 1 μM to about 1.25 mM. In some embodiments, the 2-aminopurine concentration is about 10 μM to about 1.25 mM. In some embodiments, the 2-aminopurine concentration is about 100 μM to about 1.25 mM. In some embodiments, the 2-aminopurine concentration is about 1.25 mM.

In specific embodiments, the cells of the present disclosure are contacted with 2-aminopurine about 24 hours post-infection with a recombinant adenovirus. In some embodiments, the cells of the present disclosure are contacted with 2-aminopurine about 20 hours post-infection with a recombinant adenovirus. In some embodiments, the cells of the present disclosure are contacted with 2-aminopurine about 12 hours post-infection with a recombinant adenovirus. In some embodiments, the cells of the present disclosure are contacted with 2-aminopurine about 30 hours post-infection with a recombinant adenovirus. In some embodiments, the cells of the present disclosure are contacted with 2-aminopurine about 36 hours post-infection with a recombinant adenovirus. In some embodiments, the cells of the present disclosure are contacted with 2-aminopurine about 48 hours post-infection with a recombinant adenovirus.

### EXAMPLES

The following examples of the application are to further illustrate the nature of the application. It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

### Materials

Cells: HEK293 Cells (American Type Culture Collection (ATCC), Manassas, VA, Catalog Number CRL-1573); PEAK-rapid (ATCC, Manassas, VA, Catalog Number CRL2828).

Tissue Culture Media and Reagents: OptiMEM Medium (Thermo-fisher, Waltham, MA; Catalog Number 31985-062); DMEM, high glucose (Thermo-fisher, Catalog Number 10569-010); DMEM, No Phenol Red (Thermo-fisher; Catalog Number A14430-01); Hyclone Dialyzed Fetal Bovine Serum (Thermo-fisher; Catalog Number SH30079.03); 96-well TC plate (Corning, Corning NY; Catalog Number 3596); 6-well tissue culture plates, clear (Corning Catalog #3516); Culture Plate 96, Opaque White (PerkinElmer, Waltham, MA; Catalog Number 6005680); TrypLE Select Cell disassociation reagent (Thermo-fisher, Catalog Number 12563-011); Dulbecco's Phosphate Buffered Saline, no calcium, no magnesium, D-PBS (Thermo-fisher, Catalog #14190-144); Geneticin (G418) 50 mg/ml (Thermo-fisher, Catalog Number 10131-027); Puromycin dihydrochloride from Streptomyces alboniger (Sigma Aldrich P9620); T150 tissue culture flasks 150 mm2 (Corning, Catalog Number CLS430825); GlutaMax 100× (Thermo-fisher, Catalog Number 35050-061); Non-tissue culture treated 6-well culture plates (Corning, Catalog Number 351146); Hyperflask M vessels (Corning, Catalog Number 10030); 2.5% ClonaCell methylcellulose in DMEM (without L-glutamine and contains glucose, sodium pyruvate, and sodium bicarbonate) (StemCell Technologies, Vancouver, British Columbia, Canada Catalog Number 03899-DI).

Transfection Reagents: Fugene-HD Transfection Reagent (Promega, Madison WI, Catalog Number E2311); Lipofectamine 3000 transfection reagent (Thermo-fisher Catalog Number L3000008); Deoxynucleotides (Millipore-Sigma, St. Louis, MO, Catalog Number D7295-2ML).

Tubes: 15 ml conical tubes (Corning, Catalog Number 430053); 1.5 ml screw cap tube (Sarstedt AG & Co. KG, Germany, Catalog Number 72.692.005).

Purification Kits and Assay Reagents: Plasmid Spin Miniprep kit (Qiagen, Hilden, Germany, Catalog Number 27106); CHROMA SPIN™+TE-1000 Columns (Takara Bio USA, Mountainview CA, Catalog Number 636079); Dual-Glo Luciferase Assay System (Promega, Madison WI, Catalog Number E2940); Silver staining Kit (Thermo-fisher Catalog number 24600); Trizol Plus RNA purification kit with Phase-maker tubes (Thermo-fisher Catalog Number A33254); DNA-Free Kit (Thermo-fisher Catalog Number AM1906); Nucleospin Gel and PCR Cleanup Kit (Takara Bio USA, Catalog Number 740609.5).

Enzymes: Spe I-HF (New England Biolabs, Ipswich, MA, Catalog Number R3133S); DNAse I grade II from bovine pancreas (Sigma-Aldrich, Catalog Number 10104159001); NEXT Ultra II Q5 Master Mix (New England Biolabs, Catalog Number M05445S).

Buffers and Chemicals: CutSmart® Buffer (1× Buffer Components: 50 mM Potassium Acetate, 20 mM Tris-acetate, 10 mM Magnesium Acetate, 100 g/ml BSA, pH 7.9@25° C.) (New England Biolabs, Catalog Number B7204S); Benzonase Nuclease (Sigma-Aldrich, Catalog Number E1014-25K); 10× GeneAmp PCR Buffer I containing 1.5 mM MgCl2 (Thermo-fisher Catalog Number N8080006); Sodium Deoxycholate (Sigma-Aldrich, Catalog Number D6750-25 g); 1M TRIS-HCL PH8.5 (Thermo-fisher, Catalog number T1085); 10× GeneAmp PCR Buffer I (Thermo-Fisher Catalog Number N8080006) [100 mM Tris-HCl, pH 8.3 (at 25° C.); 500 mM KCl; 15 mM MgCl2; 0.01% gelatin in autoclaved, deionized, ultrafiltered water]; 10% Pluronic F-68 (Thermo-Fisher Catalog Number 24040-032); Sheared salmon sperm DNA (10 mg/ml) (Thermo-Fisher Catalog Number AM9680); Virus Dilution Buffer (VDB) [1× GeneAmp PCR Buffer I, 2 μg/ml sheared salmon sperm DNA, and 0.05% Pluronic F-68); 0-mercaptoethanol (Sigma-Aldrich, Catalog number M3148); Adenovirus formulation buffer (10 mM Tris (pH 7.4), 1 mM MgCl2, 75 mM NaCl, 5% sucrose, 0.02% Polysorbate 80, 0.1 mM EDTA, 10 mM histidine, 0.5% EtOH); 2-Aminopurine, nitrate salt (Sigma-Aldrich, Catalog number A2380), dissolved to 100 mM in DMEM+2% FBS.

RT-PCR Reagents: SuperScript III First-Strand Synthesis System (Thermo-fisher Catalog Number 188080-051); Q5 Hot Start High-Fidelity 2× Master Mix (New England Biolabs, Catalog Number M0494S); 1% TAE Mini ReadyAgarose Gel with ethidium bromide (Bio-RAD, Catalog Number 1613016); Dark Reader Blue Light Transilluminator (Clare Chemicals, Dolores, CO, Catalog Number DR46B)

Digital Droplet PCR: 2× SuperMix for Probes (Bio-Rad Catalog Number 186-3026); DG32 AutoDG Cartridges (Bio-Rad Catalog Number 1864108); Auto Droplet Generator Oil in PBS (Bio-Rad Catalog Number 1864110); Droplet reader oil (Bio-Rad Catalog Number 1863004); Eppendorf twin.tec 96-Well PCR Plates (Catalog Number 951020346); Automated Droplet Generator (Bio-Rad Catalog number 186-4101); QX200 Droplet Reader (Bio-Rad Catalog number 186-4003); C1000Touch Thermal Cycler with Deep Well Reaction Module (Bio-Rad Catalog number 185-1197).

PrimeTime qPCR Assays: A 20× stock of these assays consist of a forward and reverse PCR primer (at 18 μM) and 5′ nuclease probe containing fluorescence quenchers ZEN and Black Hole Quencher 1 (3IABkFQ) and either FAM or HEX fluorescent Reporter Dyes (at 5 μM). Assays were synthesized by Integrated DNA Technologies, Inc., Coralville IA. Primer and Probe sequences for qPCR assays are as follows:

mCherry:
Primer 1
(SEQ ID NO: 36, 5′-CTGTTCCACGATGGTGTAGTC-3′);
Primer 2
(SEQ ID NO: 37, 5′-TGAGGTCAAGACCACCTACA-3′);
Probe
(SEQ ID NO: 38, 5′-FAM-TTGGACATC-ZEN-ACCTCCCACAACG
AG-3IABkFQ-3′);
Adenovirus Exon 2 (Ad5E2):
Primer 1
(SEQ ID NO: 39, 5′-GGGTGATGCAGTAGAAGGTAAG-3′);
Primer 2
(SEQ ID NO: 40, 5′-ATGAAGTTCGGCGGAGATG-3′);
Probe
(SEQ ID NO: 41, 5′-HEX-TC TTGTTCC-Zen-CAGCGGTCCCAT
C-3IABkFQ-3′);
P5 (P5 Promoter region of AAV):
Primer 1
(SEQ ID NO: 42, 5′-GTGGTCACGCTGGGTATTTA-3′);
Primer 2
(SEQ ID NO: 43, 5′-GGGACCTTAATCACAATCTCGT-3′);
Probe
(SEQ ID NO: 44, 5′-FAM-TTTGAAGCG-ZEN-GGAGGTTTGAACG
C-31ABkFQ-3′);
AAV REP Gene:
Primer 1
(SEQ ID NO: 45, 5′-GTCCGTGAGTGAAGCAGATATT-3′);
Primer 2
(SEQ ID NO: 46, 5′-TTCGATCAACTACGCAGACAG-3′);
Probe
(SEQ ID NO: 47, 5′-FAM-TCTGATGCT-ZEN-GTTTCCCTGCAGA
CA-3IABkFQ-3′);
AAV9 CAP Gene:
Primer 1
(SEQ ID NO: 48, 5′-CCGGGTCCAAGGTATTTGTAA-3′);
Primer 2
(SEQ ID NO: 49, 5′-CTCAACCCAAGGCAAATCAAC-3′);
Probe
(SEQ ID NO: 50, 5′-FAM-ACATCAAGA-ZEN-CAACGCTCGAGGT
CT-3IABkFQ-3′);
and
Beta lactamase (Ampicillin resistance) gene:
Primer 1
(SEQ ID NO: 51, 5′-CCAGAAACGCTGGTGAAAGTA-3′);
Primer 2
(SEQ ID NO: 52, 5′-CTCAAGGATCTTACCGCTGTTG-3′);
Probe
(SEQ ID NO: 53, 5′-FAM-TGCACGAGT-ZEN-GGGTTACATCGAA
CT-3IABkFQ-3′).

PAGE Electrophoresis: 4× NuPAGE LDS sample buffer (Thermo-Fisher, Catalog number NP0007); 4-12% Bis-Tris PAGE gel in 1×MOPS running buffer (Thermo-Fisher, Catalog number NP0322PK2); 20× NuPAGE MOPS SDS Running Buffer (Thermo-Fisher, Catalog number NP0001).

AAV Purification Buffer and Supplies: 0.2 m PES membrane filter (Thermo-Fisher Catalog number 567-0020); 0.5×5 cm POROS GoPure chromatography column, pre-packed with POROS CaptureSelect AAVX resin (Thermo-fisher Catalog Number A36652); Amicon 15 100 kDa MWCO Filter (Millipore-Sigma Catalog Number UFC910024); CIM QA Disk 0.34 ml volume (BIA Separations, Slovenia); Buffer A (20 mM Tris pH7.5, 400 mM NaCl); Buffer B (25 mM Tris pH7.5, 40 mM NaCl, and 1.5 mM MgCl2); Buffer C (20 mM Sodium Citrate pH 2.5, 400 mM NaCl); Buffer D (100 mM Sodium Citrate, 10 mM Tris, pH 8.0); Buffer E (20 mM BTP pH10.0, 0.001% Pluronic F68, 10 mM NaCl); Buffer F (20 mM Bis-TRIS Propane pH 10.0, 0.001% Pluronic F68, 400 mM NaCl); Bis-TRIS Propane (BTP) (Millipore Sigma Catalog Number B4679).

Other Equipment: AKTA Explorer FPLC system (GE Healthcare Life Sciences, Marlborough, MA); AKTA Purifier system (GE Healthcare Life Sciences); Envision multilabel reader Model 2104 (PerkinElmer, Waltham, MA).

### Identification and Recombinant Expression of SR21 Recombinase

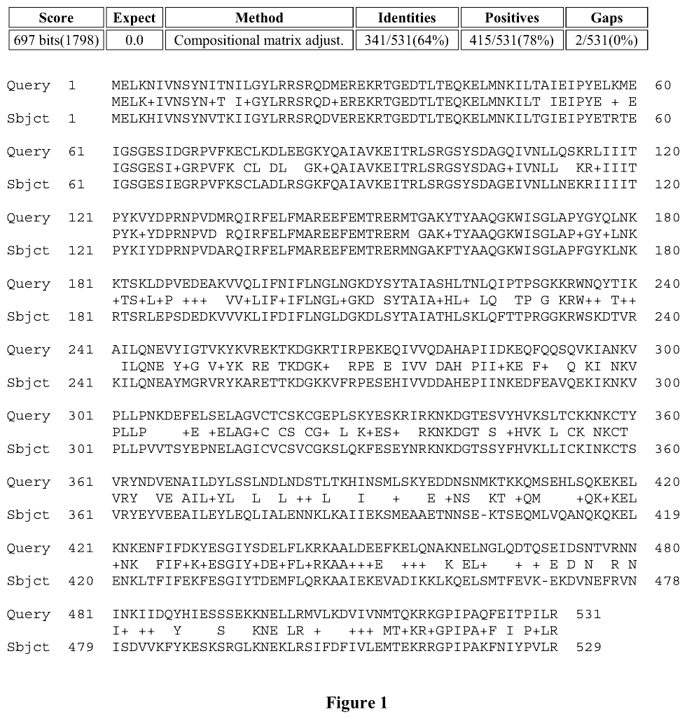

Using BLAST searches of the Non-redundant protein database at NCBI with SPBeta c2 integrase protein (Query, SEQ ID NO:1) as a query, a putative serine recombinase (Sbjct, SEQ ID NO:2) was identified in the genome of Bacillus safensis strain CCMA-560 with 64% sequence identity at the protein level (FIG. 1). The putative serine recombinase or integrase is part of a putative prophage insertion. This recombinase was named SR21 (Serine Recombinase 21). The DNA sequence encoding SR21 is shown in SEQ ID No:3.

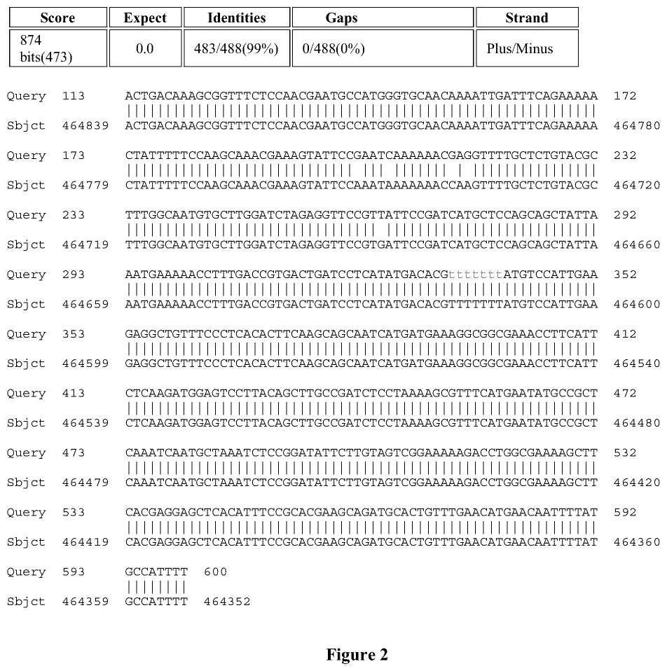

A bacterial strain that is closely related to CCMA-560 that does not contain the prophage insertion (the “Fairview” strain) was identified by BLAST searches of sequence databases at NCBI using a CCMA-560 DNA sequence from the 3′ end of the recombinase coding region and beyond as a query (SEQ ID NO:58) (FIG. 2). A DNA sequence of the Fairview strain corresponding to the upstream and downstream sequences of the putative prophage insertion site in CCMA-560 is referred to herein as the “pre-insertion sequence,” and is shown in SEQ ID NO:4. Using this sequence (SEQ ID NO:4) as a query to BLAST the genomic sequence of CCMA-560 strain identified the other Prophage-host DNA junction 94 kb upstream. The sequences of the right and left prophage-host DNA junctions of Bacillus safensis strain CCMA-560 are shown in SEQ ID NO:5 and SEQ ID NO:6, respectively.

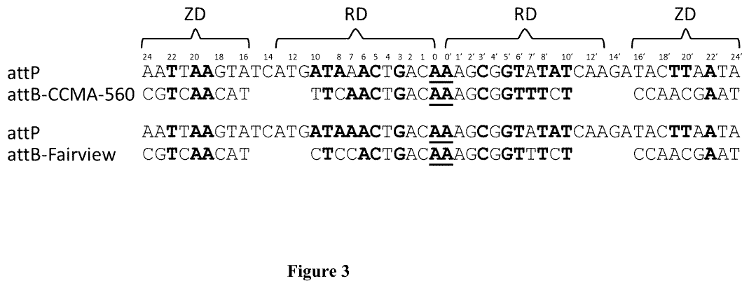

SR21 recombinase attP and attB sequences were reconstructed from the host DNA junctions (SEQ ID NO:5) and (SEQ ID NO:6), respectively by exchanging sequences upstream of a central identical region (“ACTGACAAAGCGGT”) (SEQ ID NO:54) and picking the central dinucleotide and att site boundaries that maximized the dyad symmetry: attP (SEQ ID NO:7); attB-CCMA-560 (SEQ ID NO:8). The attB sequence (SEQ ID NO:9) derived from the host DNA junction (SEQ ID NO:4) of the Fairview strain of Bacillus safensis contains two mismatches relative to the reconstructed attB sequence from strain CCMA-560 (SEQ ID NO:8). FIG. 3 shows the alignment of attP with these two alternate attB sequences, highlighting positions of dyad symmetry.

### Measuring Recombinase Activity in Mammalian Cells

A vector (P175) (SEQ ID NO:10) was constructed by Gene Synthesis (GENEWIZ, Plainfield, NJ) to express SR21 recombinase in mammalian cells under control of the CMV promoter and followed by an SV40 polyadenylation signal. The SR21 recombinase open reading frame is the same as SEQ ID NO:3 except that the bacterial translation initiation codon “TTG” is replaced by an “ATG”, and three point-mutations were introduced to destroy restriction endonuclease recognition sites. These changes in the open reading frame do not result in any change in the encoded SR21 recombinase amino acid sequence.

A recombinase reporter plasmid (P41) was also constructed by gene synthesis (GENEWIZ, Plainfield, NJ) (SEQ ID NO:11; FIG. 4). It encodes a constitutively expressed green fluorescent protein (GFP)—self-cleaving F2A—Renilla luciferase (rLUC) fusion protein driven by the EF1α promoter. It also encodes recombinase activated—mCherry-self cleaving P2A—firefly luciferase (fLUC) reporter gene flanked by SR21 attP (SEQ ID NO:7) and attB (SEQ ID NO:9) signals in the antisense orientation relative to the CMV promoter. When SR21 recombinase recombines the attP and attB signals, the coding region is inverted into the sense orientation and the mCherry-P2A-fLUC protein is expressed (See FIG. 4).

To measure SR21 recombinase activity in human cells, 75,000 HEK293 cells were plated into each well of 96-well tissue culture plates in 100 μl of high-glucose DMEM+10% Fetal Bovine Serum. The recombinase reporter plasmid (P041) ±the SR21 Recombinase expression plasmid (P175)+deoxynucleotides (to normalize DNA amounts) were complexed with Fugene-HD transfection reagent in OptiMEM medium for 15 minutes at room temperature as shown in Table 1 and transfected into triplicate wells of the plated cells. Plates were incubated at 37° C. for 48 hours.

TABLE 1
Transfection Conditions

| | | P175 | | | |
|---|---|---|---|---|---|
| | P41 | Recombinase | | | |
| | Recombinase | Expression | Deoxynucle- | Fugene- | |
| Sample | Reporter | Plasmid | otides | HD | OptiMEM |
| 1 | 4 μl | none | 12 μl | 1.2 μl | 22.8 μl |
| | (100 ng) | | (300 ng) | | |
| 2 | 4 μl | 4 μl | 8 μl | 1.2 μl | 22.8 μl |
| | (100 ng) | (100 ng) | (200 ng) | | |

Firefly Luciferase (fLUC) and Renilla luciferase (rLUC) was assayed sequentially in transfected wells using the Dual Glo assay kit from Promega. Medium was removed from the transfected wells of the tissue culture plate and 100 μl of a 1:1 mixture of DMEM medium (without phenol red) and the Dual Glo luciferase+ fLUC substrate was added. The plate was incubated at room temperature for 10 minutes. The lysate was transferred to an opaque white 96-well plate. fLUC activity was measured using the Envision multilabel reader. Next, 50 μl per well of the Stop-and-Glo buffer+Renilla substrate was added and the plate was incubated with gentle shaking for 10 minutes. The Renilla luciferase signal was read on the same Envision reader.

Results: The recombinase reporter produced 1535-fold-more firefly luciferase when co-transfected with the recombinase expression plasmid than when co-transfected with deoxynucleotides instead (Table 2). This difference is not explained by different transfection efficiencies since Renilla luciferase (rLUC) activity was 5-fold higher in the reporter alone transfection. This data demonstrates that SR21 recombinase is highly active in human cells and this result is representative of three independent experiments.

TABLE 2
Recombinase Activity in HEK293 Cells

| | | | | Fold-Increase |
|---|---|---|---|---|
| Sample | Description | fLUC | rLUC | in fLUC Activity |
| 1 | Reporter | 4.3E03 ± | 1.4E07 ± | |
| | alone | 1.2E03 | 2.6E06 | |
| 2 | Reporter + | 6.6E06 ± | 3.4E06 ± | 1535 |
| | Recombinase | 3.9E05 | 1.6E05 | |

### Constructing REP/CAP+Transgene Plasmid

Large-scale production of AAV in mammalian cells may be possible if the AAV replication (REP) and Capsid (CAP) genes could be stably integrated and later induced to produce AAV in high density cultures. However, the expression of REP proteins is toxic, making it difficult to develop stable cell lines in hosts where REP genes are expressed such as those that express the Adenovirus E1 genes such as HEK293 cells. Wild-type AAV encodes four REP proteins with overlapping reading frames that result from the use of two promoters and alternate splicing. Hence, the use of an inducible promoter to control REP expression is not straightforward. Previous work demonstrated that a “Stop Cassette” inserted into the REP coding region inside an artificial intron allowed stable cell lines to be generated in HEK293 cells (Qiao et al. (2002) J. Virol. 76: 13015; Yuan et al. (2011) Hum Gene Therap. 22: 613-624). Excision of the Stop Cassette using Cre Recombinase delivered by adenovirus infection restored REP expression and initiated AAV replication of an ITR-flanked transgene. In this example, an improved version of a Recombinase-activated REP gene in the context of a REP/CAP expression cassette in a plasmid that contained an ITR-flanked transgene was constructed.

An AAV REP/CAP9 expression cassette (SEQ ID NO:13) was constructed using the AAV2 REP gene (bp 190-2202 of human AAV2, NC_001401.2), followed by the AAV9 CAP open reading frame (AY530579.1), the AAV2 polyadenylation signal (bp 4411-4466, NC_001401.2), and a second copy of the AAV2 REP P5 promoter (bp 190-313, NC_001401.2).

Splice site prediction software (NetGene2 at www.cbs.dtu.dk/services/NetGene2/; Brunak, S., Engelbrecht, J., and Knudsen, S.: Prediction of Human mRNA Donor and Acceptor Sites from the DNA Sequence, Journal of Molecular Biology, 1991, 220, 49-65.) was used to pick a suitable location to insert an intron from the Human 3-Actin gene into the REP coding region. The intron was inserted downstream of nucleotide number 1052 in AAV2 (NC_001401.2) in a region that is common to all four REP transcripts. Both the intron and the insertion location are different than that used by Qiao et al. (2002) J. Virol. 76: 13015). The Stop Cassette (below) was subsequently inserted between the upstream and downstream halves of this β-Actin intron (SEQ IDs 14 and 15, respectively).

### STOP Cassette

The transcriptional STOP cassette (SEQ ID NO:16) was composed of the following elements:
SR21 attP (SEQ ID NO:7)
A strong splice acceptor (SEQ ID NO:17) (NC_000086.7, nucleotides 53001998 to 53002138 from the mouse HPRT gene, plus a 29 nt region from the Human Agouti Signaling protein (NC_000020.11, nucleotides 34262765 to 34262793). This was included to prevent the STOP cassette from being splice out of primary mRNA transcripts.
A neomycin phosphotransferase expression cassette (SEQ ID NO:18) was driven by a mammalian promoter (mouse phosphoglycerate kinase 1) and a bacterial (Lac zya) promoter and followed by a polyadenylation signal from SV40. This gene confers resistance to neomycin and kanamycin in mammalian and bacterial cells, respectively.
A sequence from the human β-globin gene downstream of the polyadenylation signal that encodes a self-cleaving RNA motif that is important for efficient transcription termination (Teixeira et al. (2004) Nature 432: 526-30; SEQ ID No:19).
SR21 attB (SEQ ID NO:8).

### AAV Transgene

An AAV Inverted Terminal Repeat (ITR)-flanked transgene was encoded in the P439 vector (SEQ ID NO:12) downstream of the AAV REP/CAP region. The 130 bp ITR (SEQ ID NO:20) was derived from the 3′ AAV2 ITR (Nucleotides 4535-4664, NC_001401.2) and was inserted upstream of the HPRT-E2A-mCherry transgene and reverse direction 3′ of the transgene.

The transgene consisted of the Human EF1-alpha promoter (including exon 1, intron 1, and part of exon 2) (SEQ ID NO:21), a sequence encoding a mCherry—self-splicing E2A linker—Human HPRT fusion gene (SEQ ID NO:22), and a polyadenylation signal from the Herpes Simplex Virus Thymidine Kinase Gene (SEQ ID NO:23).

### Insulators

The REP/CAP and ITR-Transgene elements were flanked by genomic elements that block chromatin-associated repression of gene expression (Kwaks et al. (2003) Nature Biotechnology 21: 554-558; Kwaks et al. (2003) Nature Biotechnology 21: 822): Human anti-repressor element 40 (AY190756.1, SEQ ID NO:24) and Mouse anti-repressor element 40 (SEQ ID NO:25).

### Plasmid Backbone

The plasmid backbone contains the following elements:
A mammalian puromycin resistance gene cassette constructed from the Herpes virus thymidine kinase gene promoter (SEQ ID NO:26), the puromycin N-acetyl transferase coding region (SEQ ID NO:27), and a polyadenylation signal from bovine growth hormone gene (SEQ ID NO:28).
An Epstein Barr Virus (EBV) OriP replication origin fragment (SEQ ID NO:29), which represents a composite of the ‘Dyad Symmetry’ region and the ‘Family of Repeats’ region of EBV
pUC57 vector sequence encoding plasmid replication origin and ampicillin resistance gene (SEQ ID NO:30).

The sequence of the complete Plasmid P439 is given in SEQ ID NO:12.

### Test Efficiency of STOP Cassette Removal by SR21 Recombinase

To test whether the Stop Cassette could be precisely removed by SR21 Recombinase in human cells, vector P439 (SEQ ID NO:12) and the SR21 Recombinase expression vector P175 (SEQ ID NO:10) were co-transfected into PEAK-Rapid cells using Lipofectamine 3000 according to manufacturer's instructions and were cultured in media containing DMEM and 10% FBS for three days at 37° C. in 5% C02. Media was removed, cells were washed once with D-PBS and then incubated with TrypLE for 5 minutes at 37° C. Cells were transferred to a sterile microfuge tube, pelleted by centrifugation, washed once with 1 ml D-PBS and pelleted again. Episomal plasmids were recovered by alkaline lysis using the Qiagen Spin Miniprep kit designed for isolating plasmids from bacteria.

To destroy unrecombined plasmid DNA, an aliquot of the recovered DNA was digested with enzyme Spe I-HF in 1× CutSmart Buffer at 37° C. 1 hour and 80° C. 20 minutes. The recovered DNA was subjected to PCR amplification with primers P349F3 (SEQ ID NO:32) and P349R9 (SEQ ID NO:33) using NEXT Ultra II Q5 Master Mix with the following cycling conditions: 98° C. 1 min; 35× (98° C. 10s, 72° C. 10 s); 5 min 72° C. A single PCR product of the predicted size was observed when subjected to electrophoresis on a 1% agarose gel. The PCR product was purified by size exclusion chromatography using a CHROMA SPIN™+TE-1000 Column. The PCR product was sequenced using the same primers used for PCR (GeneWiz). The resulting sequence (SEQ ID NO:34) demonstrated that the STOP cassette had been precisely removed from plasmid P439 by SR21 recombinase through recombining the attP (SEQ ID NO:7) and attB (SEQ ID NO:8) sequences, producing an attL recombined sequence (SEQ ID NO:35).

### Construction of Recombinant Adenovirus Serotype 5 Expressing SR21 Recombinase

Recombinant ΔE1/ΔE3 Adenovirus serotype 5 (Ad5) virus was generated at Batavia Biosciences (Leiden, the Netherlands) by a homologous recombination procedure in PER.C6 cells (Fallaux et al. (1998) Hum Gene Ther. 9: 1909-1917) as previously described for production of E1 deleted vectors (Havenga et a. (2001) J. Virol 75:3335-3342) except that a modified cosmid (pWE/Ad5.AflII-rITRsp.ΔE3, patent U.S. Pat. No. 6,340,595B1) lacking the E3 region was used. Co-expression of PER.C6 cells with this cosmid and plasmid P321 (SEQ ID NO:31) that contains the Ad5 sequence from 1 to 454 (left ITR and packaging signal), a cassette for transgene expression containing the cytomegalovirus (CMV) promoter (nt −672 to +15), the SR21 Recombinase coding region, simian virus 40 (SV40) polyadenylation signal (NC_001669.1, nt 2550 to 2774) and a second Ad5 sequence ranging from nt 3511 to 6095). Homologous recombination between the P321 Ad5 sequence (nt 3511-6095) and cosmid pWE/Ad5.AflII-rITRsp.ΔE3 in PER.C6 cells produces a recombinant adenovirus. Purified virus stocks were obtained by a two-step CsCl-gradient banding procedure and the isolated virus stocks were dialyzed into the adenovirus formulation buffer (10 mM Tris (pH 7.4), 1 mM MgCl₂, 75 mM NaCl, 5% sucrose, 0.02% Polysorbate 80, 0.1 mM EDTA, 10 mM histidine, 0.5% EtOH).

### Stable Cell Line Generation

Plasmid P439 (SEQ ID NO:12) was transfected into adherent PEAK-RAPID cells using Lipofectamine 3000 according to manufacturer's instructions and cultured in a T25 flask in DMEM+10% FBS+ 0.05 mg/ml Geneticin at 37° C. After 24 hours, cells were treated with TrypLE and transferred to a T75 flask containing DMEM+ 10% FBS+ 0.05 mg/ml Geneticin+ 0.5 μg/ml puromycin. Cells were split 1:10 weekly into the same medium for two successive weeks. At the third week post transfection, the cells were split 1:10 weekly for three weeks into media containing DMEM+ 10% FBS+ 0.05 mg/ml Geneticin+ 5.0 μg/ml puromycin.

Single-cell clones were produced by diluting cells into 1% ClonaCell Methylcellulose in DMEM+30% FBS+1× GlutaMax+ 5 μg/ml puromycin+θ0.05 mg/ml Geneticin, plating into non-tissue culture treated 6-well plates, and culturing at 37° C. for three weeks. Using a pipettor, clones were transferred from methylcellulose plates into 96-well TC-treated plates containing DMEM+10% FBS+0.05 mg/ml Geneticin+ 5.0 μg/ml puromycin. Clones were expanded in the same medium by standard methods.

### Screening Clones

To screen clones for AAV production, cells were plated in duplicate into 96-well plates in 100 μl DMEM+10% FBS and incubated overnight at 37° C. SR21 Adenovirus was diluted to 1E8 viral genomes per ml in serum-free DMEM. The media from the plated cells was replaced with 100 μl diluted adenovirus and the plate was incubated at 37° C. for four days. Cells were lysed by adding 10 μl of the following mixture: 5% Deoxycholate in PBS+10 units Benzonase. The plate was incubated at 37° C. 2 hours. The plate was centrifuged at 3000 rpm for 5 minutes to pellet cellular debris, and AAV viruses in the supernatant were quantitated by digital droplet PCR (ddPCR).

Digital Droplet PCR (ddPCR)

ddPCR quantitation was based on the method described by Lock et al. (2014) Human Gene Therapy methods 23: 115-125. Two μl of the lysate was DNAse digested in 20 μl reactions containing 1×PCR buffer+20 mM Tris pH 8.5+ 8 units DNAse I at 37° C. for 1 hour 96-well plates in a thermocycler. 2 μl of the DNAse digested samples were diluted with 98 μl Virus Dilution Buffer (VDB) and 2 μl of this dilution was added to ddPCR reactions containing 1× PCR SuperMix+1×PCR Primer/Probe for the mCherry transgene (See Materials section). ddPCR droplets were formed using the Bio-Rad automated droplet maker. PCR cycling was as follows: 95° C. 10 min; 42× (94° C. 30 s, 60° C. 1 min, 72° C. 15 s all three at cycling time of 2° C. per s); 98° C. 10 min; 4° C. hold. FAM fluorescence was detected on the Bio-Rad droplet reader as per manufacturer's instructions. The clones that produced the highest DNAse-resistant particles as detected as FAM-fluorescence positive droplets were subjected to expansion and further screening.

### Screening Clones—Second Assay

1.25E6 cells of clones to be screened were plated into single wells of a 6-well plate in 3 mls DMEM+10% FBS and incubated for 2 days at 37° C. The growth medium was replaced with 3 mls DMEM+10% FBS containing 5E8 Ad5-SR21 virus particles. Plates were returned to 37° C. to incubate for 3 days. Cells and media were transferred to 15 ml tubes and subjected to 3 freeze thaw cycles (Dry ice/37° C. incubation) followed by centrifugation at 3000 rpm for 5 minutes to pellet cellular debris. 2 μl of each sample was subjected to DNAse digestion and ddPCR quantitation with the mCherry assay as described above. P439C4 cells produced the most AAV upon infection with Ad5-SR21 virus and was selected for further characterization (Table 3).

TABLE 3
AAV Production in Screening Assays

| | Total AAV (DNAse- |
|---|---|
| Clone # | Resistant Particles) |
| clone 1 | 4.4E+08 ± 7.6E+07 |
| clone 3 | 2.5E+09 ± 3.4E+08 |
| clone 4 | 4.4E+09 ± 5.6E+07 |
| clone 5 | 1.8E+09 ± 1.9E+08 |
| clone 12 | 7.5E+08 ± 9.8E+07 |
| clone 18 | 1.6E+08 ± 2.6E+07 |
| clone 20 | 9.8E+07 ± 1.4E+07 |
| clone 25 | 1.2E+09 ± 6.4E+07 |
| clone 28 | 1.3E+08 ± 1.2E+07 |
| clone 32 | 1.5E+09 ± 1.3E+08 |
| clone 36 | 2.8E+08 ± 3.0E+07 |
| clone 41 | 1.0E+09 ± 8.9E+07 |

### Time Course Experiment

A new experiment was conducted to determine the kinetics of AAV production and secretion in the culture media at two different growth temperatures. Two mls of a non-enzymatic dissociation solution was added to PBS-washed monolayers of P439-C4 cells in T150 flasks and the flask was incubated at 37° C. for 5 minutes. Flasks were washed with 8 mls of DMEM+10% FBS and the cells were transferred to 50 ml centrifuge tube. Cells were centrifuged at 1500 rpm for 5 minutes and the pellets were resuspended in DMEM+2% FBS. Cells were diluted to 1.25E6 cells per ml in the same medium. Four mls of cell were plated into each well of four 6-well plates. 1 ml (2E8 vp) of Ad5-CMV-SR21 adenovirus in DMEM+2% FBS was added to wells. Two plates were incubated at 37° C. and two plates were incubated at 32° C. at 5% CO2. Each day for 8 days, cells and media were recovered using a cell scraper to dislodge attached cells and samples were transferred to 15 ml conical tubes. Tubes were spun for 5 minutes at 3000 rpm and an aliquot was transferred to a 1.5 ml screw cap tube and frozen at −20° C. until ddPCR assays.

Samples were DNAse treated in duplicate as described above and three serial dilutions were made in VDB for each DNAse-treated sample. Samples were quantitated in ddPCR reactions containing 1×PCR Master Mix+1× mCherry-FAM Assay+1× Ad5E2-HEX Assay (See Materials section). ddPCR was performed as described above.

### Results:

Adenovirus and AAV in the cell culture medium increased over the 8-day time course (Table 4). Adenovirus replication was slower at 32° C. resulting in higher AAV production, probably as a result of delayed adenovirus cytopathic effect. AAV production at 32° C. exceeded 14,000 genome copies per cell.

TABLE 4
AAV and Adenovirus Yields Over 8-Day Time Course

| Days | | | | |
|---|---|---|---|---|
| Post- | | | | |
| Infec- | AAV GC/Cell | | Ad5 GC/Cell | |
| tion | 32° C. | 37° C. | 32° C. | 37° C. |
| 1 | 4 ± 1 | 22 ± 0 | 8 ± 1 | 161 ± 4 |
| 2 | 11 ± 2 | 174 ± 26 | 223 ± 14 | 1319 ± 292 |
| 3 | 2009 ± 137 | 803 ± 110 | 2028 ± 155 | 4941 ± 75 |
| 4 | 4275 ± 274 | 975 | 2600 ± 168 | 5806 ± 159 |
| 5 | 7406 ± 309 | 1672 ± 49 | 5041 ± 446 | 12000 ± 442 |
| 6 | 7469 ± 75 | 4109 ± 214 | 4781 ± 610 | 33344 ± 1781 |
| 7 | 10313 ± 619 | 5034 ± 75 | 8344 ± 663 | 20594 ± 1547 |
| 8 | 14031 ± 221 | 5563 ± 88 | 13313 ± 177 | 22875 ± 442 |

### Hyperflask Cultures

8.3E07 P439C4 cells were plated into two Hyperflask M vessels in 550 mls of DMEM+10% FBS+0.5 μg/mL Puromycin, +50.0 μg/mL G418 and incubated at 37° C. for 3 days. Density after 3 days growth was estimated to be 3.6E8 cells per flask. Flasks were infected at 40 MOI (1.4E10 vp) or 20 MOI (7.2E09 vp) by diluting virus in 550 mls of DMEM+10% FBS and replacing the medium in the hyperflasks with the diluted virus. The cells were incubated at 32° C. at 5% CO2 for 7 days. Supernatants were collected from the infections after 7 days and were clarified by passing through a 0.2 m PES membrane filter.

### AAVX Purification

A 0.5×5 cm POROS GoPure chromatography column, pre-packed with POROS CaptureSelect AAVX resin to a bed volume of 1 mL, attached to an AKTA Explorer FPLC system was equilibrated with 10 column volumes (CV) buffer A (20 mM Tris pH7.5, 400 mM NaCl) at a flow rate of 3 ml/min. Virus suspension was loaded at a flow rate of 4.5 mL/min, followed by 10 mL of Buffer A to wash out unbound sample. An on-column DNA digestion was performed by equilibrating the column with 5 ml of a low salt benzonase buffer, buffer B (25 mM Tris pH7.5, 40 mM NaCl, and 1.5 mM MgCl2), then loading the column with 15 mls buffer B containing 250 units/ml Benzonase. The column was then incubated at room temperature for 30 minutes, followed by a 15 CV wash with buffer A. Virus was eluted with 15 CV buffer C (20 mM Sodium Citrate pH 2.5, 400 mM NaCl) in 0.5 mL fractions that were immediately neutralized with 25 μL of 500 mM Bis-TRIS Propane pH 10.0. Single peak elution observed. All fractions under the curve were pooled, concentrated and buffer exchanged into buffer D (100 mM Sodium Citrate, 10 mM Tris, pH 8.0) using an Amicon 15 100 kDa MWCO (Cat #UFC910024, Fisher) using three rounds of buffer addition/centrifugation. The buffer-exchanged and concentrated affinity chromatography product was subjected to anion exchange chromatography to further purify AAV away from empty capsids.

### Ion Exchange Chromatography

The affinity chromatography product (viral suspension) was diluted to 45 mL in buffer E (20 mM BTP pH10.0, 0.001% Pluronic F68, 10 mM NaCl) and loaded onto a CIM QA Disk (BIA Separations, 0.34 ml volume) at a flow rate of 2 ml/min on an AKTA Purifier system (GE Healthcare Life Sciences). Column was washed with 10 CV of sterile filtered Buffer E (20 mM BTP pH10.0, 0.001% Pluronic F68, 10 mM NaCl). Virus was eluted over a 60 CV gradient from 100% Buffer E to 100% Buffer F (20 mM Bis-TRIS Propane pH 10.0, 0.001% Pluronic F68, 400 mM NaCl), collecting 0.5 mL fractions. All fractions under the curve were pooled and concentrated using an Amicon 15 100 kDa MWCO (cat #: UFC910024, Fisher) by a 5 min centrifugation at 2000×g and buffer exchanged into buffer D (100 mM Sodium Citrate, 10 mM Tris, pH 8.0).

### Protein Visualization

2 μL of concentrated eluate was heat denatured (95° C. for 10 min) in NuPage LDS sample buffer (4×) supplemented with 5% β-mercaptoethanol and electrophoresed on 4-12% Bis-Tris PAGE gel in 1×MOPS running buffer. The gel was subjected to silver staining according to the manufacturer's instructions.

ddPCR

Viral concentration was measured by digital droplet PCR using the mCherry assay as described above.

### Results

Infection and growth of P439C4 cells in Hyperflask vessels produced 1.9E13 and 7.0E13 genome copies (GC) when infected at 20 and 40 MOI, respectively. This corresponds to 5.2E4 and 1.9E5 GC per cell for the 20 and 40 MOI infections, respectively. The purity of the virus samples was examined through PAGE electrophoresis and silver staining. Only three bands corresponding to the sizes expected for the three AAV9 capsid isoforms (VP1, VP2, and VP3) were visible (FIG. 5). The capsid proteins (VP1 (87 kDa), VP2 (72 kDa), and VP3 (62 kDa) are present in the expected stoichiometry of approximately 1:1:10 as reported previously for other recombinant AAV vectors (Daya and Berns (2008) Clin Microbiol Rev. 21: 583-593).

### Measuring the Level of Mispackaged DNA

Sequences encoding AAV REP or CAP genes and prokaryotic sequences derived from plasmid vectors used during production can be non-specifically packaged into AAV particles and represent potential safety risks when used for gene therapy (see, e.g., Schnodt and Buning, Hum Gene Ther Methods., 2017; 28(3):101-108). Risks include the generation of replication-competent AAV through homologous recombination, capsid gene expression triggers cytotoxic T lymphocyte reactions, and immune system recognition of prokaryotic sequences resulting in inflammatory responses and/or gene silencing. Encapsidated rep, cap, and prokaryotic sequences of 2%, 0.4%-1.0%, and 1.3%-6.3%, respectively have been reported in purified recombinant AAV preparations produced by triple transfection or from produced cell lines (Nony et al. (2003) J. Virology 77: 776-781; Gao et al. (2008) Molecular Therapy 16: S105; Chaudeuf et al. (2005) Molecular therapy 12: 744-753).

To determine the level of mispackaging associate with the producer system described above, the abundance of four sequences in the transfected vector (outside of the ITR-flanked transgene) was determined by ddPCR: a) the P5 promoter; b) the AAV REP gene; c) the AAV9 CAP gene; and d) the beta-lactamase (ampicillin resistance) gene. Purified virus preparations from the 20 and 40 MOI hyperflask cultures previously described were DNAse digested in triplicate, serially diluted in VDB and subjected to ddPCR. The concentration of virus particles containing these sequences were expressed as percentages of AAV particles containing the mCherry transgene (Table 5). The highest encapsidation rate of 0.04% was that of the P5 promoter in the virus A prep (produced with 20 MOI of infecting recombinant adenovirus). However, the P5 encapsidation rate in prep B (40 MOI) where AAV yield was much higher was only 0.007%. REP, CAP, and Ampicillin gene sequences were the same or lower. CAP levels were 0.007%-0.009%, which are lower than the 0.016%-0.021% cap encapsidation rate previously reported for four clinical lots of recombinant AAV2 produced for a hemophilia B gene therapy trial (Hauck et al. (2009) Molecular Therapy 17: 144-152.) Thus, the method described here for producing and purifying recombinant AAV results in a very low rate of mispackaged DNA in line with what may be required for clinical gene therapy programs.

TABLE 5
Abundance of Non-Transgene Sequences
Packaged in Purified Virus

| | P5 Promoter | REP | CAP | Ampicillin |
|---|---|---|---|---|
| Virus A | 0.0398% ± | 0.0078% ± | 0.0092% ± | 0.0051% ± |
| (20 MOI) | 0.0023% | 0.0003% | 0.0002% | 0.005% |
| Virus B | 0.0074% ± | 0.0005% ± | 0.007% ± | 0.0003% ± |
| (40 MOI) | 0.0032% | 0.0002% | 0.0001% | 0.0001% |

The mean percentage of DNAse-resistant particles ± standard deviation for four probes relative to mCherry transgene containing particles is shown for anlayses of two AAV vector preparations.

RT-PCR Analysis of RNA Splicing of the REP Gene after Stop Cassette Excision

To determine whether the intron inserted into the REP gene in construct P439 is accurately spliced when the STOP cassette is excised, an RT-PCR experiment was conducted.

Ten million cells from a stable pool of P439 in PEAK-RAPID cells were pelleted by centrifugation and resuspended in 15 mls of DMEM+2% FBS+1E9 Ad5-CMV-SR21 virus particles. Cells were plated into a T75 flask and incubated at 37° C. for forty-eight hours. Cells were detached using a cell scraper. Media and cells were transferred to a 15 ml centrifuge tube and were centrifuged at 1500 rpm for 10 minutes to pellet the cells. RNA was purified from the cell pellet using the Trizol Plus RNA purification kit with Phase-maker tubes.

To remove any contaminating DNA, 31 μg of RNA was treated with 1 μl of DNAse from the DNA-Free kit in 1× digestion buffer at 37° C. for 30 minutes. 5 μl of the DNAse inactivation slurry was added and the sample was inverted several times during a 2-minute incubation. The RNA sample was centrifuged at 10,000×g for 5 minutes and the RNA was transferred to a new sterile tube.

The RNA was reverse transcribed with the SuperScript III First Strand Synthesis System. 80 μg RNA, 1 μl 50 μM Oligo-DT, and 1 μl 10 mM dNTPS were mixed in a sterile tube and incubated at 65° C. for 5 minutes and on ice for 2 minutes. Ten μl of a 2× mixture was added (2×RT buffer, 10 mM MgCl2, 20 mM dithiothreitol, 0.5 μl RNAse-out, and 0.5 μl Reverse Transcriptase). Mock RT reactions were identical except that reverse transcriptase was replaced with water. The reactions were incubated at 50° C. for 50 min and on ice for 2 minutes. One μl RNAse H was added and samples were incubated for 20 minutes at 37° C.

Fifty μl PCR reactions contained 1 μl of reverse transcribed RNA, 25 μl Q5 Hot Start High-Fidelity 2× Master Mix, and 0.5 μM of two primers. Reactions were subjected to thermocycling as follows:
98° C. 1 min; 35 cycles of (98° C. 10 s, 69° C. 10 s, 72° C. 36 s); 5 min 72° C.

Five μl of the reactions were resolved on 1% Agarose gels in 1×TAE buffer and ethidium bromide. Bands were visualized under blue light illumination on the Dark Reader transilluminator. DNA was recovered from excised bands using the Nucleospin gel and PCR cleanup kit. DNA was sequenced at GeneWiz (South Plainfield, NJ) with the PCR primers using cycle-sequencing and dye-terminator chemistry.

### Results:

PCR reactions from mock-RT templates did not produce detectable products, indicating that genomic DNA had been eliminated from RNA samples. PCR using primers AAVRT-F1 (SEQ ID NO:62) and P349R9 (SEQ ID NO:63) produced two PCR products of similar fluorescent intensity derived from spliced transcripts after the STOP cassette has been excised from P439. One product resulted from splicing at the engineered beta-Actin splice donor and acceptor sites (SEQ ID NO:14 and SEQ ID NO:15 respectively; FIG. 8). The second product results from splicing between a donor site in the 5′ REP gene (SEQ ID NO:64) and the downstream beta-Actin acceptor (FIG. 8). This splicing event is predicted to remove 64 bp of the REP coding sequence relative to wild-type AAV2, creating a frameshift and producing truncated REP proteins. This suggests that mutating this upstream splice donor site could increase the abundance of active REP proteins and make AAV production more efficient.

### Updating the AAV Construct: P600 (SEQ ID NO:70)

Several changes were made to plasmid P439 (SEQ ID NO:12), resulting in construct P600 (SEQ ID NO:70). First, the splice donor site of the REP gene upstream of the STOP cassette was mutated. Briefly, the nucleotides GT of the splice donor site identified in the 5′ REP sequence (SEQ ID NO:64) was mutated to AT (SEQ ID NO:65). This mutation is predicted to eliminate splicing at this site without changing the REP protein sequence.

To reduce the possibility that the REP/CAP gene could be packaged into AAV capsids following excision of the STOP cassette, a 2 kb random sequence (SEQ ID NO:66) was designed and inserted downstream of the attB sequence and upstream of the Actin splice acceptor to increase the size of the engineered intron. Potential splice sites were identified using NetGene 2 software (Cited above) and removed. This insertion increased the size of the REP/CAP gene from 4.3 kb to 6.4 kb, which is well above the 5.0 kb AAV packaging limit.

Based on the hypothesis that sequences adjacent to AAV ITRs may also be amplified during transgene rescue from the genome and might be mispackaged into AAV capsids (see, e.g., Schnodt and Buning, Hum Gene Ther Methods., 2017; 28(3):101-108), two, random 2 kb non-coding spacer elements were design to flank the transgene to decrease the potential impact of mispackaged DNA. One element (SEQ ID NO:67) was inserted upstream of the left AAV ITR and the second (SEQ ID NO:68) replaced mouse anti-repressor element 40 (SEQ ID NO:25) downstream of the right AAV ITR.

In addition, the cap gene was an AAV9 variant (see, e.g., Hinderer et al., Hum Gene Ther. 2018; 29(3):285-298).

Finally, the coding sequence of the ITR-flanked transgene in P439 was replaced by SEQ ID NO:69 encoding an mCherry-IRES-SEAP (secreted alkaline phosphatase) protein.

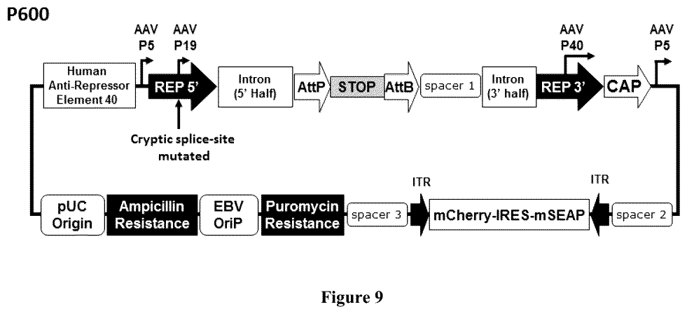

The complete sequence of the resulting construct P600 is disclosed in SEQ ID NO:70 and an illustration of the plasmid is shown in FIG. 9.

AAV Production from P600 in Stable Pools

Construct P600 was transfected into Peak-RAPID cells and a stable pool was generated through selection with 0.5 μg/ml puromycin essentially as described for P439 cells above. Cells were passed 1:10 for 6 weeks prior to assaying AAV production. 2.5E6 P600-PEAK-Rapid p6 cells were plated into three T25 flasks in 5 mls DMEM+10% FBS and incubated for three days at 37° C. The cells density on the day of infection was determined to be 6.6E6 viable cells in one of the flasks where cells were recovered with TrypLE and counted using Trypan blue exclusion. Media in the two remaining flasks were replaced with 11 mls DMEM+2% FBS containing 2.6E8 Ad5-CMV-SR21 virus particles. The flasks were incubated for 24 hours at 32° C. One ml of 1.25 mM 2-Aminopurine in DMEM+2% FBS was added to one flask. One ml of DMEM+2% FBS was added to the other flask, and both flasks were incubated at 32° C. for 7 additional days. Media was recovered from the flasks, centrifuged at 3,000 rpm for 5 minutes to pellet cells and debris. 2 μl of each sample supernatant was subjected to DNAse digestion and ddPCR quantitation using the mCherry assay as described above.

AAV production levels are shown in Table 6. The P600 stable pool is active in producing AAV upon infection with Ad5-CMV-SR21. AAV virus production was increased 2.5-fold in the presence of 2-aminopurine, a drug reported to block adenovirus-induced inhibition of CAP-dependent mRNA translation (see, e.g., Zhang and Schneider (1994) J. Virology 68: 2544-2555; and Huang and Schneider (1990) PNAS 87: 7115-7119). While it has been reported that 10 mM 2-AP treatment 1-2 hours post infection blocked cytopathic effect of adenovirus infection and was nontoxic for at least three days (see, e.g., Zhang and Schneider (1994) J. Virology 68: 2544-2555; and Huang and Schneider (1990) PNAS 87: 7115-7119), we found concentrations above 1.25 mM and addition earlier than 24 hours to be inhibitory to AAV production in our AAV producer cell system. These data suggest that inhibiting late adenoviral gene programs, especially the shutdown of cap-dependent mRNA translation, is a useful strategy for increasing AAV production in producer cell lines.

TABLE 6

| Sample | Media | AAV GC/Cell |
|---|---|---|
| 1 | DMEM + 2% FBS | 23,826 ± 2990 |
| 2 | DMEM + 2% FBS + 1.25 | 59,356 ± 11,026 |
| | mM2-AP | |

Manufacturing of recombinant adeno-associated virus (AAV) in human cells requires expression of AAV replication (REP) and capsid (CAP) genes, adenovirus genes and an AAV-packageable transgene consisting of an expression cassette flanked by AAV inverted terminal repeats (ITRs). All three components can be delivered to cells on separate plasmids for AAV production, but existing transfection methods are difficult to scale to large-scale cultures. Incorporating some of these elements into the host cell line could make AAV production more efficient, however, some of the AAV and adenovirus genes are cytostatic or cytotoxic, limiting this approach. The present invention describes a way to reversibly-inactivate the AAV REP genes such that AAV REP, CAP, and a packageable transgene can be integrated into suitable host cells and expanded. Infection of these cells by a replication-deficient recombinant adenovirus (e.g., ΔE1/ΔE3) expressing a recombinase reactivates the REP genes and induces AAV replication and packaging.

What is claimed is:

1. A non-naturally occurring nucleic acid molecule comprising a modified adeno-associated virus (AAV) rep gene having an AAV rep gene encoding four Rep proteins Rep78, Rep68, Rep52 and Rep40 and an artificial intron inserted into a coding sequence of the rep gene shared by the four Rep proteins, wherein the artificial intron comprises a stop cassette inserted downstream of the 5′ splice site and upstream of the branch site of the artificial intron, and the stop cassette comprises, in 5′ to 3′ order:
(a) an attP site having a nucleotide sequence at least 90%, 91%, 92%, 93%, 94%, 95%, 96%, 97%, 98%, 99% or 100%, identical to SEQ ID NO:7, preferably, an attP site having the nucleotide sequence of SEQ ID NO:7;
(b) a splice acceptor;
(c) a terminator; and
(d) an attB site having a nucleotide sequence at least 90%, 91%, 92%, 93%, 94%, 95%, 96%, 97%, 98%, 99% or 100%, identical of SEQ ID NO:8 or SEQ ID NO:9, preferably, an attB site having the nucleotide sequence of SEQ ID NO:8 or SEQ ID NO:9.

2. The non-naturally occurring nucleic acid molecule of claim 1, wherein the splice acceptor comprises the nucleotide sequence of SEQ ID NO:17.

3. The non-naturally occurring nucleic acid molecule of claim 1 or 2, wherein the terminator comprises a polyadenylation signal.

4. The non-naturally occurring nucleic acid molecule of claim 3, wherein the terminator further comprises the nucleotide sequence of SEQ ID NO:19.

5. The non-naturally occurring nucleic acid molecule of any one of claims 1 to 4, wherein the stop cassette comprises a gene encoding a selectable marker, preferably a neomycin phosphotransferase expression cassette having the nucleotide sequence of SEQ ID NO:18.

6. The non-naturally occurring nucleic acid molecule of any one of claims 1 to 5, wherein the artificial intron comprises, in 5′ to 3′ order, the nucleotide sequence of SEQ ID NO:14, the stop cassette, and the nucleotide sequence of SEQ ID NO:15.

7. The non-naturally occurring nucleic acid molecule of any one of claims 1 to 6, wherein the AAV rep gene comprises a rep gene of one of AAV1 to AAV8, or a hybrid thereof.

8. The non-naturally occurring nucleic acid molecule of claim 7, wherein the AAV rep gene comprises the rep gene of human AAV2 having nucleotide numbers 190 to 2202 of the nucleotide sequence of GenBank accession number NC_001401.2.

9. The non-naturally occurring nucleic acid molecule of claim 8, wherein the artificial intron is inserted between nucleotide numbers 996 to 1905 of the nucleotide sequence of GenBank accession number NC_001401.2.

10. The non-naturally occurring nucleic acid molecule of claim 9, wherein the artificial intron is inserted immediately downstream of nucleotide number 1052, 1061, 1712, 1906, 1022, 1112, 1475, 1514, 1700, 1742, 1784 or 1340, preferably nucleotide number 1052, of the nucleotide sequence of GenBank accession number NC_001401.2.

11. A non-naturally occurring nucleic acid molecule comprising a modified AAV rep gene comprising, in 5′ to 3′ order:
(a) a 5′ portion of an AAV rep gene having the nucleotide sequence of SEQ ID NO:55;
(b) an artificial intron comprising, in 5′ to 3′ order:
(i) a 5′ intron fragment having the nucleotide sequence of SEQ ID NO:14;
(ii) a stop cassette comprising, in 5′ to 3′ order:
(1) an attP site having the nucleotide sequence of SEQ ID NO:7;
(2) a splice acceptor having the nucleotide sequence of SEQ ID NO:17;
(3) a neomycin phosphotransferase expression cassette having the nucleotide sequence of SEQ ID NO:18;
(4) a terminator having the nucleotide sequence of SEQ ID NO:19; and
(5) an attB site having the nucleotide sequence of SEQ ID NO:8 or SEQ ID NO:9; and
(iii) a 3′ intron fragment having the nucleotide sequence of SEQ ID NO:15; and
(c) a 3′ portion of the AAV rep gene having the nucleotide sequence of SEQ ID NO:56.

12. A non-naturally occurring nucleic acid molecule comprising a modified AAV rep gene comprising, in 5′ to 3′ order:
(a) a 5′ portion of an AAV rep gene having the nucleotide sequence of SEQ ID NO:73;
(b) an artificial intron comprising, in 5′ to 3′ order:
(i) a 5′ intron fragment having the nucleotide sequence of SEQ ID NO:14;
(ii) a stop cassette comprising, in 5′ to 3′ order:
(1) an attP site having the nucleotide sequence of SEQ ID NO:7;
(2) a splice acceptor having the nucleotide sequence of SEQ ID NO:17;
(3) a neomycin phosphotransferase expression cassette having the nucleotide sequence of SEQ ID NO:18;
(4) a terminator having the nucleotide sequence of SEQ ID NO:19; and
(5) an attB site having the nucleotide sequence of SEQ ID NO:8 or SEQ ID NO:9; and
(iii) a 3′ intron fragment having the nucleotide sequence of SEQ ID NO:66; and
(c) a 3′ portion of the AAV rep gene having the nucleotide sequence of SEQ ID NO:56.

13. The non-naturally occurring nucleic acid molecule of claim 11 or 12, wherein the stop cassette comprises the nucleotide sequence of SEQ ID NO:16.

14. The non-naturally occurring nucleic acid molecule of any one of claims 1 to 13, further comprising an AAV cap gene encoding three capsid proteins VP1, VP2 and VP3.

15. The non-naturally occurring nucleic acid molecule of claim 14, wherein the AAV cap gene comprises a cap gene of one of AAV1 to AAV9 and AAVDJ, or a hybrid thereof.

16. The non-naturally occurring nucleic acid molecule of claim 15, wherein the AAV cap gene comprises the cap gene of human AAV9 having the nucleotide sequence of GenBank accession number AY530579.1.

17. The non-naturally occurring nucleic acid molecule of any one of claims 14 to 16, wherein the AAV cap gene further comprises a polyadenylation signal, preferably a polyadenylation signal of AAV2 having nucleotide numbers 4411 to 4466 of the nucleotide sequence of GenBank accession number NC_001401.2, and an enhancer, preferably an AAV2 rep P5 promoter having nucleotide numbers 190 to 313 of the nucleotide sequence of GenBank accession number NC_001401.2, wherein the polyadenylation signal and the enhancer are both downstream of the coding sequence of the cap gene.

18. The non-naturally occurring nucleic acid molecule of any one of claims 14 to 17, further comprising a transgene flanked by a pair of AAV inverted terminal repeats (ITRs) downstream of the AAV cap gene.

19. The non-naturally occurring nucleic acid molecule of claim 18, further comprising a first insulator upstream of the modified AAV rep gene and optionally a second insulator downstream of the transgene flanked by the ITRs, preferably, the first insulator and the second insulator are independently selected from the group consisting of:
(a) a human anti-repressor element 40 having the nucleotide sequence of SEQ ID NO:24;
(b) a mouse anti-repressor element 40 having the nucleotide sequence of SEQ ID NO:25;
(c) an anti-repressor element 04 having the nucleotide sequence of GenBank accession number AY190749.1;
(d) an anti-repressor element 06 having the nucleotide sequence of GenBank accession number AY190750.1;
(e) an anti-repressor element 07 having the nucleotide sequence of GenBank accession number AY190751.1;
(f) an anti-repressor element 12 having the nucleotide sequence of GenBank accession number AY190752.1;
(g) an anti-repressor element 13 having the nucleotide sequence of GenBank accession number AY190753.1;
(h) an anti-repressor element 35 having the nucleotide sequence of GenBank accession number AY190754.1;
(i) an anti-repressor element 36 having the nucleotide sequence of GenBank accession number AY190755.1;
(j) an anti-repressor element 52 having the nucleotide sequence of GenBank accession number AY190757.1;
(k) an anti-repressor element 53 having the nucleotide sequence of GenBank accession number AY190758.1; and
(l) a Chicken HS4 insulator from the globin locus having the nucleotide sequence of AY040835.1 in two or more copies,
more preferably, the first insulator and the second insulator have the nucleotide sequences of SEQ ID NO:24 and SEQ ID NO:25, respectively.

20. The non-naturally occurring nucleic acid molecule of claim 19, wherein the non-naturally occurring nucleic acid molecule comprises the first insulator upstream of the modified AAV rep gene, and further comprises a first spacer sequence and a second spacer sequence upstream and downstream of the transgene, respectively, wherein the first spacer sequence and the second spacer sequence are independently selected from the group consisting of:
(a) a nucleotide sequence of SEQ ID NO:67; and
(b) a nucleotide sequence of SEQ ID NO:68.

21. The non-naturally occurring nucleic acid molecule of any one of claims 18 to 20, wherein the ITR has the nucleotide sequence of SEQ ID NO:20, the transgene comprises a promoter operably linked to a coding sequence, and the coding sequence is operably linked a polyadenylation signal; preferably, the promoter has the nucleotide sequence of SEQ ID NO:21 and the polyadenylation signal has the nucleotide sequence SEQ ID NO:23.

22. A non-naturally occurring nucleic acid molecule comprising, in 5′ to 3′ order:
(A) a first insulator, preferably the first insulator has the nucleotide sequence of SEQ ID NO:24;
(B) a modified AAV rep gene comprising, in 5′ to 3′ order:
(i) a 5′ portion of an AAV rep gene, preferably the 5′ portion of the AAV rep gene has the nucleotide sequence of SEQ ID NO:55;
(ii) an artificial intron comprising, in 5′ to 3′ order:
(a) a 5′ intron fragment, preferably the 5′ intron fragment has the nucleotide sequence of SEQ ID NO:14;
(b) a stop cassette comprising, in 5′ to 3′ order:
(1) an attP site having the nucleotide sequence of SEQ ID NO:7;
(2) a splice acceptor, preferably the splice acceptor has the nucleotide sequence of SEQ ID NO:17;
(3) a gene encoding a selectable marker, preferably a neomycin phosphotransferase expression cassette having the nucleotide sequence of SEQ ID NO:18;
(4) a terminator, preferably the terminator has the nucleotide sequence of SEQ ID NO:19; and
(5) an attB site having the nucleotide sequence of SEQ ID NO:8 or SEQ ID NO:9; and
(c) a 3′ intron fragment, preferably the 3′ intron fragment has the nucleotide sequence of SEQ ID NO:15;
(iii) a 3′ portion of the AAV rep gene, preferably the 3′ portion of the AAV rep gene has the nucleotide sequence of SEQ ID NO:56;
(C) an AAV cap gene, preferably the AAV cap gene comprises the nucleotide sequence of SEQ ID NO:57;
(D) a transgene flanked by a pair of AAV ITRs, preferably, the AAV ITR has the nucleotide sequence of SEQ ID NO:20, and the transgene comprises a promoter operably linked to a coding sequence, and the coding sequence is operably linked a polyadenylation signal; more preferably, the promoter has the nucleotide sequence of SEQ ID NO:21 and the polyadenylation signal has the nucleotide sequence SEQ ID NO:23; and
(E) a second insulator, preferably the second insulator has the nucleotide sequence of SEQ ID NO:25.

23. A non-naturally occurring nucleic acid molecule comprising, in 5′ to 3′ order:
(A) a first insulator, preferably the first insulator has the nucleotide sequence of SEQ ID NO:24;
(B) a modified AAV rep gene comprising, in 5′ to 3′ order:
(i) a 5′ portion of an AAV rep gene, preferably the 5′ portion of the AAV rep gene has the nucleotide sequence of SEQ ID NO:73;
(ii) an artificial intron comprising, in 5′ to 3′ order:
(a) a 5′ intron fragment, preferably the 5′ intron fragment has the nucleotide sequence of SEQ ID NO:14;
(b) a stop cassette comprising, in 5′ to 3′ order:
(1) an attP site having the nucleotide sequence of SEQ ID NO:7;
(2) a splice acceptor, preferably the splice acceptor has the nucleotide sequence of SEQ ID NO:17;
(3) a gene encoding a selectable marker, preferably a neomycin phosphotransferase expression cassette having the nucleotide sequence of SEQ ID NO:18;
(4) a terminator, preferably the terminator has the nucleotide sequence of SEQ ID NO:19; and
(5) an attB site having the nucleotide sequence of SEQ ID NO:8 or SEQ ID NO:9; and
(c) a 3′ intron fragment, preferably the 3′ intron fragment has the nucleotide sequence of SEQ ID NO:66;
(iii) a 3′ portion of the AAV rep gene, preferably the 3′ portion of the AAV rep gene has the nucleotide sequence of SEQ ID NO:56;
(C) an AAV cap gene;
(D) a transgene flanked by
(1) a pair of AAV ITRs, preferably, the AAV ITR has the nucleotide sequence of SEQ ID NO:20, and the transgene comprises a promoter operably linked to a coding sequence, and the coding sequence is operably linked a polyadenylation signal; more preferably, the promoter has the nucleotide sequence of SEQ ID NO:21 and the polyadenylation signal has the nucleotide sequence SEQ ID NO:23; and
(2) a pair of spacer sequences, preferably, the spacer sequences have a nucleotide sequence of SEQ ID NO:67 and SEQ ID NO:68.

24. A vector comprising the non-naturally occurring nucleic acid molecule of any one of claims 1 to 22; preferably, the vector is a plasmid; more preferably, the plasmid comprises the nucleotide sequence of SEQ ID NO:12.

25. A vector comprising the non-naturally occurring nucleic acid molecule of any one of claims 1 to 21 or 23; preferably, the vector is a plasmid; more preferably, the plasmid comprises the nucleotide sequence of SEQ ID NO:70.

26. A method of making the non-naturally occurring nucleic acid molecule of any one of claims 1 to 23.

27. A method of making the vector of claim 24 or 25.

28. A cell comprising a non-naturally occurring nucleic acid molecule comprising a modified adeno-associated virus (AAV) rep gene having an AAV rep gene encoding four Rep proteins Rep78, Rep68, Rep52 and Rep40 and an artificial intron inserted into a coding sequence of the rep gene shared by the four Rep proteins, wherein the artificial intron comprises a stop cassette inserted downstream of the 5′ splice site and upstream of the branch site of the artificial intron, and the stop cassette comprises, in 5′ to 3′ order:
(a) an attP site having a nucleotide sequence at least 90%, 91%, 92%, 93%, 94%, 95%, 96%, 97%, 98%, 99% or 100%, identical to SEQ ID NO:7, preferably, an attP site having the nucleotide sequence of SEQ ID NO:7;
(b) a splice acceptor;
(c) a terminator; and
(d) an attB site having a nucleotide sequence at least 90%, 91%, 92%, 93%, 94%, 95%, 96%, 97%, 98%, 99% or 100%, identical of SEQ ID NO:8 or SEQ ID NO:9, preferably, an attB site having the nucleotide sequence of SEQ ID NO:8 or SEQ ID NO:9.

29. The cell of claim 28, wherein the splice acceptor comprises the nucleotide sequence of SEQ ID NO:17.

30. The cell of claim 28 or 29, wherein the terminator comprises a polyadenylation signal.

31. The cell of claim 30, wherein the terminator further comprises the nucleotide sequence of SEQ ID NO:19.

32. The cell of any one of claims 28 to 31, wherein the stop cassette comprises a gene encoding a selectable marker, preferably a neomycin phosphotransferase expression cassette having the nucleotide sequence of SEQ ID NO:18.

33. The cell of any one of claims 28 to 32, wherein the artificial intron comprises, in 5′ to 3′ order, the nucleotide sequence of SEQ ID NO:14, the stop cassette, and the nucleotide sequence of SEQ ID NO:15.

34. The cell of any one of claims 28 to 32, wherein the artificial intron comprises, in 5′ to 3′ order, the nucleotide sequence of SEQ ID NO:14, the stop cassette, and the nucleotide sequence of SEQ ID NO:66.

35. The cell of any one of claims 28 to 33, wherein the AAV rep gene comprises a rep gene of one of AAV1 to AAV8, or a hybrid thereof.

36. The cell of claim 35, wherein the AAV rep gene comprises the rep gene of human AAV2 having nucleotide numbers 190 to 2202 of the nucleotide sequence of GenBank accession number NC_001401.2.

37. The cell of claim 36, wherein the artificial intron is inserted between nucleotide numbers 996 to 1905 of the nucleotide sequence of GenBank accession number NC_001401.2.

38. The cell of claim 37, wherein the artificial intron is inserted immediately downstream of nucleotide number 1052, 1061, 1712, 1906, 1022, 1112, 1475, 1514, 1700, 1742, 1784 or 1340, preferably nucleotide number 1052, of the nucleotide sequence of GenBank accession number NC_001401.2.

39. A cell comprising a non-naturally occurring nucleic acid molecule comprising a modified AAV rep gene comprising, in 5′ to 3′ order:
(a) a 5′ portion of an AAV rep gene having the nucleotide sequence of SEQ ID NO:55;
(b) an artificial intron comprising, in 5′ to 3′ order:
(i) a 5′ intron fragment having the nucleotide sequence of SEQ ID NO:14;
(ii) a stop cassette comprising, in 5′ to 3′ order:
(1) an attP site having the nucleotide sequence of SEQ ID NO:7;
(2) a splice acceptor having the nucleotide sequence of SEQ ID NO:17;
(3) a neomycin phosphotransferase expression cassette having the nucleotide sequence of SEQ ID NO:18;
(4) a terminator having the nucleotide sequence of SEQ ID NO:19; and
(5) an attB site having the nucleotide sequence of SEQ ID NO:8 or SEQ ID NO:9; and
(iii) a 3′ intron fragment having the nucleotide sequence of SEQ ID NO:15; and
(c) a 3′ portion of the AAV rep gene having the nucleotide sequence of SEQ ID NO:56.

40. A cell comprising a non-naturally occurring nucleic acid molecule comprising a modified AAV rep gene comprising, in 5′ to 3′ order:
(a) a 5′ portion of an AAV rep gene having the nucleotide sequence of SEQ ID NO:73;
(b) an artificial intron comprising, in 5′ to 3′ order:
(i) a 5′ intron fragment having the nucleotide sequence of SEQ ID NO:14;
(ii) a stop cassette comprising, in 5′ to 3′ order:
(1) an attP site having the nucleotide sequence of SEQ ID NO:7;
(2) a splice acceptor having the nucleotide sequence of SEQ ID NO:17;
(3) a neomycin phosphotransferase expression cassette having the nucleotide sequence of SEQ ID NO:18;
(4) a terminator having the nucleotide sequence of SEQ ID NO:19; and
(5) an attB site having the nucleotide sequence of SEQ ID NO:8 or SEQ ID NO:9; and
(iii) a 3′ intron fragment having the nucleotide sequence of SEQ ID NO:66; and
(c) a 3′ portion of the AAV rep gene having the nucleotide sequence of SEQ ID NO:56.

41. The cell of claim 39 or 40, wherein the stop cassette comprises the nucleotide sequence of SEQ ID NO:16.

42. The cell of any one of claims 28 to 41, further comprising an AAV cap gene encoding three capsid proteins VP1, VP2 and VP3.

43. The cell of claim 42, wherein the AAV cap gene comprises a cap gene of one of AAV1 to AAV9 and AAVDJ, or a hybrid thereof.

44. The cell of claim 43, wherein the AAV cap gene comprises the cap gene of human AAV9 having the nucleotide sequence of GenBank accession number AY530579.1.

45. The cell of claim 43, wherein the AAV cap gene comprises the cap gene of a hybrid of AAV9.

46. The cell of claims 42 to 45, wherein the AAV cap gene further comprises a polyadenylation signal, preferably a polyadenylation signal of AAV2 having nucleotide numbers 4411 to 4466 of the nucleotide sequence of GenBank accession number NC_001401.2, and an enhancer, preferably an AAV2 rep P5 promoter having nucleotide numbers 190 to 313 of the nucleotide sequence of GenBank accession number NC_001401.2, wherein the polyadenylation signal and the enhancer are both downstream of the coding sequence of the cap gene.

47. The cell of any one of claims 42 to 46, further comprising a transgene flanked by a pair of AAV inverted terminal repeats (ITRs) downstream of the AAV cap gene.

48. The cell of claim 47, further comprising a first insulator upstream of the modified AAV rep gene and optionally a second insulator downstream of the transgene flanked by the ITRs, preferably, the first insulator and the second insulator are independently selected from the group consisting of:
(a) a human anti-repressor element 40 having the nucleotide sequence of SEQ ID NO:24;
(b) a mouse anti-repressor element 40 having the nucleotide sequence of SEQ ID NO:25;
(c) an anti-repressor element 04 having the nucleotide sequence of GenBank accession number AY190749.1;
(d) an anti-repressor element 06 having the nucleotide sequence of GenBank accession number AY190750.1;
(e) an anti-repressor element 07 having the nucleotide sequence of GenBank accession number AY190751.1;
(f) an anti-repressor element 12 having the nucleotide sequence of GenBank accession number AY190752.1;
(g) an anti-repressor element 13 having the nucleotide sequence of GenBank accession number AY190753.1;
(h) an anti-repressor element 35 having the nucleotide sequence of GenBank accession number AY190754.1;
(i) an anti-repressor element 36 having the nucleotide sequence of GenBank accession number AY190755.1;
(j) an anti-repressor element 52 having the nucleotide sequence of GenBank accession number AY190757.1;
(k) an anti-repressor element 53 having the nucleotide sequence of GenBank accession number AY190758.1; and
(l) a Chicken HS4 insulator from the globin locus having the nucleotide sequence of AY040835.1 in two or more copies,
more preferably, the first insulator and the second insulator have the nucleotide sequences of SEQ ID NO:24 and SEQ ID NO:25, respectively.

49. The cell of claim 48, wherein the cell comprises the first insulator upstream of the modified AAV rep gene, and further comprises a first spacer sequence and a second spacer sequence upstream and downstream of the transgene, respectively, wherein the first spacer sequence and the second spacer sequence are independently selected from the group consisting of:
(a) a nucleotide sequence of SEQ ID NO:67; and
(b) a nucleotide sequence of SEQ ID NO:68.

50. The cell of any one of claims 47 to 49, wherein the ITR has the nucleotide sequence of SEQ ID NO:20, the transgene comprises a promoter operably linked to a coding sequence, and the coding sequence is operably linked a polyadenylation signal;
preferably, the promoter has the nucleotide sequence of SEQ ID NO:21 and the polyadenylation signal has the nucleotide sequence SEQ ID NO:23.

51. A cell comprising a non-naturally occurring nucleic acid molecule comprising, in 5′ to 3′ order:
(A) a first insulator, preferably the first insulator has the nucleotide sequence of SEQ ID NO:24;
(B) a modified AAV rep gene comprising, in 5′ to 3′ order:
(i) a 5′ portion of an AAV rep gene, preferably the 5′ portion of the AAV rep gene has the nucleotide sequence of SEQ ID NO:55;
(ii) an artificial intron comprising, in 5′ to 3′ order:
(a) a 5′ intron fragment, preferably the 5′ intron fragment has the nucleotide sequence of SEQ ID NO:14;
(b) a stop cassette comprising, in 5′ to 3′ order:
(1) an attP site having the nucleotide sequence of SEQ ID NO:7;
(2) a splice acceptor, preferably the splice acceptor has the nucleotide sequence of SEQ ID NO:17;
(3) a gene encoding a selectable marker, preferably a neomycin phosphotransferase expression cassette having the nucleotide sequence of SEQ ID NO:18;
(4) a terminator, preferably the terminator has the nucleotide sequence of SEQ ID NO:19; and
(5) an attB site having the nucleotide sequence of SEQ ID NO:8 or SEQ ID NO:9; and
(c) a 3′ intron fragment, preferably the 3′ intron fragment has the nucleotide sequence of SEQ ID NO:15;
(iii) a 3′ portion of the AAV rep gene, preferably the 3′ portion of the AAV rep gene has the nucleotide sequence of SEQ ID NO:56;
(C) an AAV cap gene, preferably the AAV cap gene comprises the nucleotide sequence of SEQ ID NO:57;
(D) a transgene flanked by a pair of AAV ITRs, preferably, the AAV ITR has the nucleotide sequence of SEQ ID NO:20, and the transgene comprises a promoter operably linked to a coding sequence, and the coding sequence is operably linked a polyadenylation signal; more preferably, the promoter has the nucleotide sequence of SEQ ID NO:21 and the polyadenylation signal has the nucleotide sequence SEQ ID NO:23; and
(E) a second insulator, preferably the second insulator has the nucleotide sequence of SEQ ID NO:25.

52. A cell comprising a non-naturally occurring nucleic acid molecule comprising, in 5′ to 3′ order:
(A) a first insulator, preferably the first insulator has the nucleotide sequence of SEQ ID NO:24;
(B) a modified AAV rep gene comprising, in 5′ to 3′ order:
(i) a 5′ portion of an AAV rep gene, preferably the 5′ portion of the AAV rep gene has the nucleotide sequence of SEQ ID NO:73;
(ii) an artificial intron comprising, in 5′ to 3′ order:
(a) a 5′ intron fragment, preferably the 5′ intron fragment has the nucleotide sequence of SEQ ID NO:14;
(b) a stop cassette comprising, in 5′ to 3′ order:
(1) an attP site having the nucleotide sequence of SEQ ID NO:7;
(2) a splice acceptor, preferably the splice acceptor has the nucleotide sequence of SEQ ID NO:17;
(3) a gene encoding a selectable marker, preferably a neomycin phosphotransferase expression cassette having the nucleotide sequence of SEQ ID NO:18;
(4) a terminator, preferably the terminator has the nucleotide sequence of SEQ ID NO:19; and
(5) an attB site having the nucleotide sequence of SEQ ID NO:8 or SEQ ID NO:9; and
(c) a 3′ intron fragment, preferably the 3′ intron fragment has the nucleotide sequence of SEQ ID NO:66;
(iii) a 3′ portion of the AAV rep gene, preferably the 3′ portion of the AAV rep gene has the nucleotide sequence of SEQ ID NO:56;
(C) an AAV cap gene; and
(D) a transgene flanked by
(i) a pair of AAV ITRs, preferably, the AAV ITR has the nucleotide sequence of SEQ ID NO:20, and the transgene comprises a promoter operably linked to a coding sequence, and the coding sequence is operably linked a polyadenylation signal; more preferably, the promoter has the nucleotide sequence of SEQ ID NO:21 and the polyadenylation signal has the nucleotide sequence SEQ ID NO:23; and
(ii) a pair of spacer sequences, preferably, the spacer sequences have a nucleotide sequence of SEQ ID NO:67 and SEQ ID NO:68.

53. The cell of any one of claims 28 to 51, wherein the non-naturally occurring nucleic acid molecule is episomal, having the nucleotide sequence of SEQ ID NO:12.

54. The cell of any one of claims 28 to 50 or 52, wherein the non-naturally occurring nucleic acid molecule is episomal, having the nucleotide sequence of SEQ ID NO:70.

55. The cell of any one of claims 51 to 54, further comprising a nucleic acid molecule encoding a recombinase having the amino acid sequence at least 85%, 86%, 87%, 88%, 89%, 90%, 91%, 92%, 93%, 94%, 95%, 96%, 97%, 98%, 99%, or 100% identical to the amino acid sequence of SEQ ID NO:2; preferably, the nucleic acid comprises the nucleotide sequence at least 85%, at least 85%, 86%, 87%, 88%, 89%, 90%, 91%, 92%, 93%, 94%, 95%, 96%, 97%, 98%, 99%, or 100% identical to the nucleotide sequence of SEQ ID NO:3; more preferably, the cell comprises a recombinant ΔE1/ΔE3 adenovirus serotype 5 (Ad5) virus encoding the recombinase having the amino acid sequence of SEQ ID NO:2.

56. The cell of any one of claims 51 to 55, further comprising adenovirus E1A and E1B genes, preferably the cell is a 911 cell, a pTG6559 cell, a GH329 cell, a N52.E6 cell, a HeLa-E1 cell, an UR cell, a VLI-293 cell, a HEK293 cell, or a PER.C6 cell.

57. A method of producing a recombinant AAV comprising a transgene, comprising:
(A) obtaining a first host cell comprising:
(i) a modified AAV rep gene comprising, in 5′ to 3′ order:
(a) a 5′ portion of an AAV rep gene, preferably the AAV rep gene has the nucleotide sequence of SEQ ID NO:55;
(b) an artificial intron comprising, in 5′ to 3′ order:
(1) a 5′ intron fragment, preferably the 5′ intron fragment has the nucleotide sequence of SEQ ID NO:14;
(2) a stop cassette comprising, in 5′ to 3′ order:
(aa) an attP site having the nucleotide sequence of SEQ ID NO:7;
(bb) a splice acceptor, preferably the splice acceptor has the nucleotide sequence of SEQ ID NO:17;
(cc) a gene encoding a selectable marker, preferably a neomycin phosphotransferase expression cassette having the nucleotide sequence of SEQ ID NO:18;
(dd) a terminator, preferably the terminator has the nucleotide sequence of SEQ ID NO:19; and
(ee) an attB site having the nucleotide sequence of SEQ ID NO:8 or SEQ ID NO:9; and
(3) a 3′ intron fragment, preferably the 3′ intron fragment has the nucleotide sequence of SEQ ID NO:15;
(c) a 3′ portion of the AAV rep gene, preferably the 3′ portion of the AAV rep gene has the nucleotide sequence of SEQ ID NO:56;
(ii) an AAV cap gene, preferably the AAV cap gene comprises the nucleotide sequence of SEQ ID NO:57; and
(iii) the transgene flanked by a pair of AAV ITRs, preferably, the ITR has the nucleotide sequence of SEQ ID NO:20, the transgene comprises a promoter operably linked to a coding sequence, and the coding sequence is operably linked a polyadenylation signal; more preferably, the promoter has the nucleotide sequence of SEQ ID NO:21 and the polyadenylation signal has the nucleotide sequence SEQ ID NO:23;
(B) infecting the first host cell with a recombinant adenovirus comprising a recombinase gene encoding a recombinase having at least 85%, 86%, 87%, 88%, 89%, 90%, 91%, 92%, 93%, 94%, 95%, 96%, 97%, 98%, 99%, or 100%, sequence identity to the amino acid sequence of SEQ ID NO:2 to obtain a second host cell further containing the recombinase gene;
(C) growing the second host cell under conditions in which the recombinant AAV comprising the transgene is produced; and
(D) optionally collecting the recombinant AAV.

58. A method of producing a recombinant AAV comprising a transgene, comprising:
(A) obtaining a first host cell comprising:
(i) a modified AAV rep gene comprising, in 5′ to 3′ order:
(a) a 5′ portion of an AAV rep gene, preferably the AAV rep gene has the nucleotide sequence of SEQ ID NO:73;
(b) an artificial intron comprising, in 5′ to 3′ order:
(1) a 5′ intron fragment, preferably the 5′ intron fragment has the nucleotide sequence of SEQ ID NO:14;
(2) a stop cassette comprising, in 5′ to 3′ order:
(aa) an attP site having the nucleotide sequence of SEQ ID NO:7;
(bb) a splice acceptor, preferably the splice acceptor has the nucleotide sequence of SEQ ID NO:17;
(cc) a gene encoding a selectable marker, preferably a neomycin phosphotransferase expression cassette having the nucleotide sequence of SEQ ID NO:18;
(dd) a terminator, preferably the terminator has the nucleotide sequence of SEQ ID NO:19; and
(ee) an attB site having the nucleotide sequence of SEQ ID NO:8 or SEQ ID NO:9; and
(3) a 3′ intron fragment, preferably the 3′ intron fragment has the nucleotide sequence of SEQ ID NO:66;
(c) a 3′ portion of the AAV rep gene, preferably the 3′ portion of the AAV rep gene has the nucleotide sequence of SEQ ID NO:66;
(ii) an AAV cap gene; and
(iii) the transgene flanked by:
(a) a pair of AAV ITRs, preferably, the ITR has the nucleotide sequence of SEQ ID NO:20, the transgene comprises a promoter operably linked to a coding sequence, and the coding sequence is operably linked a polyadenylation signal; more preferably, the promoter has the nucleotide sequence of SEQ ID NO:21 and the polyadenylation signal has the nucleotide sequence SEQ ID NO:23; and
(b) a pair of spacer sequences, preferably, the spacer sequences have a nucleotide sequence of SEQ ID NO:67 and SEQ ID NO:68;
(B) infecting the first host cell with a recombinant adenovirus comprising a recombinase gene encoding a recombinase having at least 85%, 86%, 87%, 88%, 89%, 90%, 91%, 92%, 93%, 94%, 95%, 96%, 97%, 98%, 99%, or 100%, sequence identity to the amino acid sequence of SEQ ID NO:2 to obtain a second host cell further containing the recombinase gene;
(C) growing the second host cell under conditions in which the recombinant AAV comprising the transgene is produced; and
(D) optionally collecting the recombinant AAV.

59. The method of claim 57 or 58, wherein the first host cell further comprises a first insulator upstream of the modified AAV rep gene and optionally a second insulator downstream of the transgene flanked by the ITRs, preferably, the first insulator and the second insulator are independently selected from the group consisting of:
(a) a human anti-repressor element 40 having the nucleotide sequence of SEQ ID NO:24;
(b) a mouse anti-repressor element 40 having the nucleotide sequence of SEQ ID NO:25;
(c) an anti-repressor element 04 having the nucleotide sequence of GenBank accession number AY190749.1;
(d) an anti-repressor element 06 having the nucleotide sequence of GenBank accession number AY190750.1;
(e) an anti-repressor element 07 having the nucleotide sequence of GenBank accession number AY190751.1;
(f) an anti-repressor element 12 having the nucleotide sequence of GenBank accession number AY190752.1;
(g) an anti-repressor element 13 having the nucleotide sequence of GenBank accession number AY190753.1;
(h) an anti-repressor element 35 having the nucleotide sequence of GenBank accession number AY190754.1;
(i) an anti-repressor element 36 having the nucleotide sequence of GenBank accession number AY190755.1;
(j) an anti-repressor element 52 having the nucleotide sequence of GenBank accession number AY190757.1;
(k) an anti-repressor element 53 having the nucleotide sequence of GenBank accession number AY190758.1; and
(l) a Chicken HS4 insulator from the globin locus having the nucleotide sequence of AY040835.1 in two or more copies,
more preferably, the first insulator and the second insulator have the nucleotide sequences of SEQ ID NO:24 and SEQ ID NO:25, respectively.

60. The method of claim 59, wherein the first host cell comprises the first insulator upstream of the modified AAV rep gene, and further comprises a first spacer sequence and a second spacer sequence upstream and downstream of the transgene, respectively, wherein the first spacer sequence and the second spacer sequence are independently selected from the group consisting of:
(a) a nucleotide sequence of SEQ ID NO:67; and
(b) a nucleotide sequence of SEQ ID NO:68.

61. The method of any one of claims 57 to 59, wherein the first host cell is obtained by introducing into a cell one or more nucleic acid molecules comprising the modified AAV rep gene, the AAV cap gene, the transgene flanked by the ITRs, the first insulator and the second insulator.

62. The method of claim 61, wherein the first host cell is obtained by introducing into the cell a nucleic acid molecule comprising, in 5′ to 3′ order, the first insulator, the modified AAV rep gene, the AAV cap gene, the transgene flanked by the ITRs, the first insulator and the second insulator, preferably, a plasmid comprising the nucleotide sequence of SEQ ID NO:12.

63. The method of claim 57, 58, or 60, wherein the first host cell is obtained by introducing into a cell one or more nucleic acid molecules comprising the modified AAV rep gene, the AAV cap gene, the transgene flanked by the ITRs, the first insulator, the first spacer sequence, and the second spacer sequence.

64. The method of claim 63, wherein the first host cell is obtained by introducing into a cell one or more nucleic acid molecules comprising the modified AAV rep gene, the AAV cap gene, the transgene flanked by the ITRs, the first insulator, the first spacer sequence, and the second spacer sequencer, preferably, a plasmid comprising the nucleotide sequence of SEQ ID NO:70.

65. The method of any one of claims 57 to 62, wherein the recombinant adenovirus is a recombinant ΔE1/ΔE3 adenovirus serotype 5 (Ad5) virus comprising a nucleotide sequence of SEQ ID NO:3.

66. The method of any one of claims 57 to 65, wherein the host cell comprises adenovirus E1A and E1B genes, preferably the host cell is a 911 cell, pTG6559 cell, GH329 cell, N52.E6 cell, HeLa-E1 cell, UR cell, VLI-293 cell, HEK293 cell, or a PER.C6 cell.

67. The method of any one of claims 57 to 66, wherein said conditions for growing the second host cell comprise culturing the second cell with 2-aminopurine.

68. The method of claim 67, wherein the 2-aminopurine concentration is less than about 1.25 mM.

69. The method of claim 67 or 68, wherein the 2-aminopurine concentration is about 1 μM to about 1.25 mM.

70. The method of claim 67 or 68, wherein the 2-aminopurine concentration is about 10 μM to about 1.25 mM.

71. The method of claim 67 or 68, wherein the 2-aminopurine concentration is about 100 μM to about 1.25 mM.

72. The method of claim 67 or 68, wherein the 2-aminopurine concentration is about 1.25 mM.

73. The method of any one of claims 67 to 72, wherein culturing the second cell with 2-aminopurine is initiated about 24 hours post-infection with the first host cell with a recombinant adenovirus.

74. A composition comprising the cell of claim 55, and 2-aminopurine.

75. The composition of claim 74, wherein the 2-aminopurine concentration is less than about 1.25 mM.

76. The composition of claim 74, wherein the 2-aminopurine concentration is about 1 μM to about 1.25 mM.

77. The composition of claim 74, wherein the 2-aminopurine concentration is about 10 μM to about 1.25 mM.

78. The composition of claim 74, wherein the 2-aminopurine concentration is about 100 μM to about 1.25 mM.

79. The composition of claim 74, wherein the 2-aminopurine concentration is about 1.25 mM.

80. A non-naturally occurring nucleic acid molecule comprising a nucleotide sequence encoding a serine recombinase having an amino acid sequence having at least 85%, 86%, 87%, 88%, 89%, 90%, 91%, 92%, 93%, 94%, 95%, 96%, 97%, 98%, 99% or 100%, identity to the amino acid sequence of SEQ ID NO:2.

81. The non-naturally occurring nucleic acid molecule of claim 74, comprising a nucleotide sequence having at least 85%, 86%, 87%, 88%, 89%, 90%, 91%, 92%, 93%, 94%, 95%, 96%, 97%, 98%, 99% or 100%, identity to the nucleotide sequence of SEQ ID NO:3.

82. A vector comprising the non-naturally occurring nucleic acid of claim 80 or 81.

83. The vector of claim 82, further comprising a promoter, preferably a cytomegalovirus (CMV) promoter operably linked to the nucleotide sequence encoding the serine recombinase.

84. The vector of claim 82 or 83, further comprising a polyadenylation signal, such as a simian virus 40 (SV40) polyadenylation signal, operably linked to the nucleotide sequence encoding the serine recombinase.

85. The vector of any one of claims 82 to 84, being a DNA plasmid.

86. The vector of any one of claims 82 to 85, being a recombinant adenoviral vector.

87. The vector of claim 86, being a recombinant ΔE1/ΔE3 adenovirus serotype 5 (Ad5) virus comprising a nucleotide sequence encoding a serine recombinase having the amino acid sequence of SEQ ID NO:2 under the control of a CMV promoter, wherein the nucleotide sequence is further operably linked to a SV40 polyadenylation signal (NC_001669.1, nt 2550 to 2774).

88. A cell comprising a non-naturally occurring nucleic acid molecule comprising a nucleotide sequence encoding a serine recombinase having an amino acid sequence having at least 85%, such as at least 85%, 86%, 87%, 88%, 89%, 90%, 91%, 92%, 93%, 94%, 95%, 96%, 97%, 98%, 99% or 100%, identity to the amino acid sequence of SEQ ID NO:2.

89. The cell of claim 88, comprising a nucleotide sequence having at least 85%, such as at least 85%, 86%, 87%, 88%, 89%, 90%, 91%, 92%, 93%, 94%, 95%, 96%, 97%, 98%, 99% or 100%, identity to the nucleotide sequence of SEQ ID NO:3.

90. A cell comprising a vector comprising the non-naturally occurring nucleic acid of claim 88 or 89.

91. The cell of claim 90, further comprising a promoter, preferably a cytomegalovirus (CMV) promoter operably linked to the nucleotide sequence encoding the serine recombinase.

92. The cell of claim 90 or 91, further comprising a polyadenylation signal, such as a simian virus 40 (SV40) polyadenylation signal, operably linked to the nucleotide sequence encoding the serine recombinase.

93. The cell of claims 90 to 92, wherein the vector is a DNA plasmid.

94. The cell of any one of claims 90 to 93, wherein the vector is a recombinant adenoviral vector.

95. The cell of claim 94, wherein the recombinant adenoviral vector is a recombinant ΔE1/ΔE3 adenovirus serotype 5 (Ad5) virus comprising a nucleotide sequence encoding a serine recombinase having the amino acid sequence of SEQ ID NO:2 under the control of a CMV promoter, wherein the nucleotide sequence is further operably linked to a SV40 polyadenylation signal (NC_001669.1, nt 2550 to 2774).

96. The cell of claims 88 to 95, comprising adenovirus E1A and E1B genes, preferably the cell is a 911 cell, pTG6559 cell, GH329 cell, N52.E6 cell, HeLa-E1 cell, UR cell, VLI-293 cell, HEK293 cell, or a PER.C6 cell.

97. A method of conducting a site-specific recombination in a cell, comprising:
(a) obtaining a cell comprising a nucleic acid molecule having an attP site having a nucleotide sequence at least 90%, 91%, 92%, 93%, 94%, 95%, 96%, 97%, 98%, 99% or 100%, identical to SEQ ID NO:7, preferably, an attP site having the nucleotide sequence of SEQ ID NO:7, and an attB site having a nucleotide sequence at least 90%, 91%, 92%, 93%, 94%, 95%, 96%, 97%, 98%, 99% or 100%, identical of SEQ ID NO:8 or SEQ ID NO:9, preferably, an attB site having the nucleotide sequence of SEQ ID NO:8 or SEQ ID NO:9;
(b) introducing to the cell a non-naturally occurring nucleic acid molecule encoding a serine recombinase having at least 85%, 86%, 87%, 88%, 89%, 90%, 91%, 92%, 93%, 94%, 95%, 96%, 97%, 98%, 99% or 100% identity, to SEQ ID NO:2; and
(c) growing the cell under conditions to allow the serine recombinase to catalyze the site-specific recombination between the attP and attB sites.

98. A product produced by the process of conducting a site-specific recombination in a cell, comprising:
(a) obtaining a cell comprising a nucleic acid molecule having an attP site having a nucleotide sequence at least 90%, 91%, 92%, 93%, 94%, 95%, 96%, 97%, 98%, 99% or 100%, identical to SEQ ID NO:7, preferably, an attP site having the nucleotide sequence of SEQ ID NO:7, and an attB site having a nucleotide sequence at least 90%, 91%, 92%, 93%, 94%, 95%, 96%, 97%, 98%, 99% or 100%, identical of SEQ ID NO:8 or SEQ ID NO:9, preferably, an attB site having the nucleotide sequence of SEQ ID NO:8 or SEQ ID NO:9;
(b) introducing to the cell a non-naturally occurring nucleic acid molecule encoding a serine recombinase having at least 85%, 86%, 87%, 88%, 89%, 90%, 91%, 92%, 93%, 94%, 95%, 96%, 97%, 98%, 99% or 100% identity, to SEQ ID NO:2; and
(c) growing the cell under conditions to allow the serine recombinase to catalyze the site-specific recombination between the attP and attB sites.

99. A process for obtaining a product from a cell, comprising:
(a) obtaining a cell comprising a nucleic acid molecule having an attP site having a nucleotide sequence at least 90%, 91%, 92%, 93%, 94%, 95%, 96%, 97%, 98%, 99% or 100%, identical to SEQ ID NO:7, preferably, an attP site having the nucleotide sequence of SEQ ID NO:7, and an attB site having a nucleotide sequence at least 90%, 91%, 92%, 93%, 94%, 95%, 96%, 97%, 98%, 99% or 100%, identical of SEQ ID NO:8 or SEQ ID NO:9, preferably, an attB site having the nucleotide sequence of SEQ ID NO:8 or SEQ ID NO:9;
(b) introducing to the cell a non-naturally occurring nucleic acid molecule encoding a serine recombinase having at least 85%, 86%, 87%, 88%, 89%, 90%, 91%, 92%, 93%, 94%, 95%, 96%, 97%, 98%, 99% or 100% identity, to SEQ ID NO:2;
(c) growing the cell under conditions to allow the serine recombinase to catalyze the site-specific recombination between the attP and attB sites; and
(d) producing and recovering from the cell a product.

100. A non-naturally occurring system, comprising:
a means for AAV mediated recombination, wherein the means optionally comprises a transgenic element.

101. A means for transferring the non-naturally occurring system of claim 100.

102. A non-naturally occurring system, comprising:
a recombination means for recombining the system of claim 100, wherein the recombination means includes using at least one serine residue during catalysis.

103. A means for transferring the non-naturally occurring system of claim 102.

104. A means for manufacturing a molecule, wherein the means for manufacturing a molecule comprises the means of any one of claims 100 to 103 and is capable of replication.

105. A process for AAV mediated site-specific recombination, comprising:
(a) a step for performing a function of obtaining a cell comprising the means of claim 100;
(b) a step for performing a function of growing the cell under conditions to allow site-specific recombination using at least one serine residue during catalysis.

106. The process for AAV mediated site-specific recombination of claim 105, comprising obtaining a product, wherein, optionally the product is a therapeutic product.


SEQUENCE LISTING (73 sequences)

SEQ ID NO: 1 - protein, 545 aa, synthetic construct
  source 1..545: note = SPBetac2 Integrase (NP_046553.1) Protein Sequence
MELKNIVNSYNITNILGYLRRSRQDMEREKRTGEDTLTEQKELMNKILTAIEIPYELKMEIGSGESIDGRPVFKECLKDLEEGKYQAIAVKEITRLSRGSYSDAGQIVNLLQSKRLIIITPYKVYDPRNPVDMRQIRFELFMAREEFEMTRERMTGAKYTYAAQGKWISGLAPYGYQLNKKTSKLDPVEDEAKVVQLIFNIFLNGLNGKDYSYTAIASHLTNLQIPTPSGKKRWNQYTIKAILQNEVYIGTVKYKVREKTKDGKRTIRPEKEQIVVQDAHAPIIDKEQFQQSQVKIANKVPLLPNKDEFELSELAGVCTCSKCGEPLSKYESKRIRKNKDGTESVYHVKSLTCKKNKCTYVRYNDVENAILDYLSSLNDLNDSTLTKHINSMLSKYEDDNSNMKTKKQMSEHLSQKEKELKNKENFIFDKYESGIYSDELFLKRKAALDEEFKELQNAKNELNGLQDTQSEIDSNTVRNNINKIIDQYHIESSSEKKNELLRMVLKDVIVNMTQKRKGPIPAQFEITPILRFNFIFDLTATNSFH

SEQ ID NO: 2 - protein, 535 aa, synthetic construct
  source 1..535: note = SR21 Recombinase (WP_029708089.1) Protein Sequence
MELKHIVNSYNVTKIIGYLRRSRQDVEREKRTGEDTLTEQKELMNKILTGIEIPYETRTEIGSGESIEGRPVFKSCLADLRSGKFQAIAVKEITRLSRGSYSDAGEIVNLLNEKRIIIITPYKIYDPRNPVDARQIRFELFMAREEFEMTRERMNGAKFTYAAQGKWISGLAPFGYKLNKRTSRLEPSDEDKVVVKLIFDIFLNGLDGKDLSYTAIATHLSKLQFTTPRGGKRWSKDTVRKILQNEAYMGRVRYKARETTKDGKKVFRPESEHIVVDDAHEPIINKEDFEAVQEKIKNKVPLLPVVTSYEPNELAGICVCSVCGKSLQKFESEYNRKNKDGTSSYFHVKLLICKINKCTSVRYEYVEEAILEYLEQLIALENNKLKAIIEKSMEAAETNNSEKTSEQMLVQANQKQKELENKLTFIFEKFESGIYTDEMFLQRKAAIEKEVADIKKLKQELSMTFEVKEKDVNEFRVNISDVVKFYKESKSRGLKNEKLRSIFDFIVLEMTEKRRGPIPAKFNIYPVLRIPSVVN

SEQ ID NO: 3 - DNA, 1608 bp, synthetic construct
  source 1..1608: mol_type = other DNA; note = SR21 Recombinase DNA Sequence
ttggaattaaaacacattgtcaattcctataatgtgactaaaattattggctatttgagacgttctagacaagatgttgaaagagagaagcgtactggtgaagatactttgacagaacaaaaagaacttatgaataaaatattgactgggattgaaataccgtacgaaacaagaactgagatcggttctggtgaaagcattgaaggaagaccagtgtttaaaagttgcttagctgatctgaggtctggaaaatttcaagcaattgctgtaaaagaaataactcgtcttagtcgtggtagttacagtgatgctggtgaaatcgtcaatcttctaaatgaaaaaaggattatcataattacaccatataaaatatatgatccaagaaatcctgttgatgctcgtcaaattagatttgagctatttatggctagagaagaatttgaaatgacaagagaaagaatgaatggtgccaagtttacttatgctgcacaaggaaaatggatttctggtttagcaccatttggctataaattaaataagcgtacatctcgcctcgaaccatcggatgaagacaaagttgtggtaaagttgatatttgacattttcttaaatggccttgatggtaaagatttaagttatacagctattgcaactcatctttctaaacttcaattcactaccccaaggggaggaaaaagatggagtaaggacactgttagaaaaattcttcaaaatgaagcctatatgggaagggttaggtataaagcaagagaaaccacaaaagatggaaaaaaagtcttccgacctgaaagtgaacatattgttgtagatgatgcccacgaaccaataataaataaagaggactttgaagctgttcaagaaaagattaaaaataaagtgcctctgcttccagtcgtaacatcttatgaacctaatgaattagcgggaatatgtgtatgctctgtctgtggaaaatcacttcaaaaatttgaatccgaatacaataggaaaaacaaagatgggacttccagctattttcacgtgaagttattgatttgcaaaatcaacaagtgcacatcagtgagatatgagtatgttgaagaagcaattttggagtatcttgagcagttgattgctttagaaaataataaactaaaggctataattgaaaaatctatggaagcagcagaaactaacaattcagaaaagacaagtgagcaaatgctagtacaagcaaaccaaaaacagaaagagctcgaaaacaaattgacatttatttttgaaaaatttgaatctggaatttatacagatgaaatgtttttgcaaaggaaagcagctattgaaaaagaagtagctgacattaaaaaattgaaacaagaacttagcatgacatttgaagttaaagagaaagatgttaatgaatttagggttaacatatcagatgtagttaaattttataaagaatctaaaagtagaggattaaaaaatgaaaagttgagatccatttttgactttatagtattagaaatgacagagaaaagacgaggccccatccctgctaagttcaatatttatccggttttgcgaattccgagtgtagttaattaa

SEQ ID NO: 4 - DNA, 188 bp, synthetic construct
  source 1..188: mol_type = other DNA; note = Bacillus safensis Fairview strain Pre-insertion locus (gi|640668059:c464939-464752)
agataatccgttcgaagctgttcaaacgaatatcatcggcgggcagcacgtcattgaagccgcaattgaacatgaagtgagtcatgtcgtcaacatctccactgacaaagcggtttctccaacgaatgccatgggtgcaacaaaattgatttcagaaaaactatttttccaagcaaacgaaagtattc

SEQ ID NO: 5 - DNA, 120 bp, synthetic construct
  source 1..120: mol_type = other DNA; note = Right Prophage-Host DNA Junction, Bacillus safensis strain CCMA-560
tttgcgaattccgagtgtagttaattaagtatcatgataaactgacaaagcggtttctccaacgaatgccatgggtgcaacaaaattgatttcagaaaaactatttttccaagcaaacga

SEQ ID NO: 6 - DNA, 158 bp, synthetic construct
  source 1..158: mol_type = other DNA; note = Left Prophage-Host DNA Junction, Bacillus safensis strain CCMA-560
tatcatcggcgggcagcacgtcattgaagccgctctcgaacatgaagtgagtcatgtcgtcaacatttcaactgacaaagcggtatatcaagatacttaatacatgataacaaaaaaagaagcctacatattggcttcttttataattatatttgcta

SEQ ID NO: 7 - DNA, 50 bp, synthetic construct
  source 1..50: mol_type = other DNA; note = SR21 Recombinase AttP reconstructed from Bacillus safensis strain CCMA-560 (artificial sequence)
aattaagtatcatgataaactgacaaagcggtatatcaagatacttaata

SEQ ID NO: 8 - DNA, 40 bp, synthetic construct
  source 1..40: mol_type = other DNA; note = SR21 Recombinase AttB reconstructed from Bacillus safensis strain CCMA-560
cgtcaacatttcaactgacaaagcggtttctccaacgaat

SEQ ID NO: 9 - DNA, 40 bp, synthetic construct
  source 1..40: mol_type = other DNA; note = SR21 Recombinase AttB reconstructed from Bacillus safensis strain Fairview
cgtcaacatctccactgacaaagcggtttctccaacgaat

SEQ ID NO: 10 - DNA, 4532 bp, synthetic construct
  source 1..4532: mol_type = other DNA; note = P175- SR21 Mammalian expression vector
taactataacggtcctaaggtagcgaagctcttcagatggacagtcagactgaagagcctctcttaaggtagctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcggcgcgccgccaccatggaattaaaacacattgtcaattcctataatgtgactaaaattattggctatttgagacgttccagacaagatgttgaaagagagaagcgtactggtgaagatactttgacagaacaaaaagaacttatgaataaaatattgactgggattgaaataccgtacgaaacaagaactgagatcggttctggtgaaagcattgaaggaagaccagtgtttaaaagttgcttagctgatctgaggtctggaaaatttcaagcaattgctgtaaaagaaataactcgtcttagtcgtggtagttacagtgatgctggtgaaatcgtcaatcttctaaatgaaaaaaggattatcataattacaccatataaaatatatgatccaagaaatcctgttgatgctcgtcaaattagatttgagctatttatggctagagaagaatttgaaatgacaagagaaagaatgaatggtgccaagtttacttatgctgcacaaggaaaatggatttctggtttagcaccatttggctataaattaaataagcgtacatctcgcctcgaaccatcggatgaagacaaagttgtggtaaagttgatatttgacattttcttaaatggccttgatggtaaagatttaagttatacagctattgcaactcatctttctaaacttcaattcactaccccaaggggaggaaaaagatggagtaaggacactgttagaaaaattcttcaaaatgaagcctatatgggaagggttaggtataaagcaagagaaaccacaaaagatggaaaaaaagtcttccgacctgaaagtgaacatattgttgtagatgatgcccacgaaccaataataaataaagaggactttgaagctgttcaagaaaagattaaaaataaagtgcctctgcttccagtcgtaacatcttatgaacctaatgaattagcgggaatatgtgtatgctctgtctgtggaaaatcacttcaaaaatttgaatccgaatacaataggaaaaacaaagatgggacttccagctattttcacgtgaagttattgatttgcaaaatcaacaagtgcacatcagtgagatatgagtatgttgaagaagcaattttggagtatcttgagcagttgattgctttagaaaataataaactaaaggctataattgaaaaatctatggaagcagcagaaactaacaattcagaaaagacaagtgagcaaatgctagtacaagcaaaccaaaaacagaaagaactcgaaaacaaattgacatttatttttgaaaaatttgaatctggaatttatacagatgaaatgtttttgcaaaggaaagcagctattgaaaaagaagtagctgacattaaaaaattgaaacaagaacttagcatgacatttgaagttaaagagaaagatgttaatgaatttagggttaacatatcagatgtagttaaattttataaagaatctaaaagtagaggattaaaaaatgaaaagttgagatccatttttgactttatagtattagaaatgacagagaaaagacgaggccccatccctgctaagttcaatatttatccggttttgcgaatcccgagtgtagttaattaatctagagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcgcggccgcaataaagatcccgggtagggataacagggtaatgctcttcagatggacagtcagactgaagagctggcaaacagctattatgggtattatgggtgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 11 - DNA, 9555 bp, synthetic construct
  source 1..9555: mol_type = other DNA; note = P41- SR21 recombinase reporter plasmid
taactataacggtcctaaggtagcgaagctcttcagatggacagtcagactgaagagcctctcttaaggtagctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcggcgcgcccgtcaacatctccactgacaaagcggtttctccaacgaatggatccttacacggcgatcttgccgcccttcttggccttaatgagaatctcgcggatcttgcgggcgtccaacttgccggtcagtcctttaggcacctcgtccacgaacacaacaccaccgcgcagcttcttggcggttgtaacctggctggccacatagtccacgatctccttctcggtcatggttttaccgtgttccagcacgacgactgcggcgggcagctcgccggcatcgtcgtcgggcaggccggcgaccccggcgtcgaagatgttggggtgttgcagcaggatgctctccagttcggctggggctacctggtagcccttgtatttgatcaggctcttcagccggtccacgatgaagaagtgctcgtcctcgtcccagtaggcgatgtcgccgctgtgcagccagccgtccttgtcgatgagagcgtttgtagcctcggggttgttaacgtagccgctcatgatcatggggccacggacgcacagctcgccgcgctggttcacacccagtgtcttaccggtgtccaagtccaccaccttagcctcgaagaagggcaccaccttgcctactgcgccaggcttgtcgtccccttcgggggtgatcagaatggcgctggttgtttctgtcaggccgtagccctggcggatgcctggtaggtggaagcgtttggccacggcctcacctacctccttgctgagcggcgccccgccgctggcgatctcgtgcaagttgcttaggtcgtacttgtcgatgagagtgctcttagcgaagaagctaaatagtgtgggcaccagcagggcagattgaatcttatagtcttgcaagctgcgcaagaatagctcctcctcgaagcggtacatgagcacgacccgaaagccgcagatcaagtagcccagcgtggtgaacatgccgaagccgtggtgaaatggcaccacgctgaggatagcggtgtcggggatgatctggttgccgaagatggggtcgcgggcatgactgaatcggacacaagcggtgcggtgcggtagggctacgcccttgggcaatccggtactgccactactgttcatgatcagggcgatggttttgtcccggtcgaagctctcgggcacgaagtcgtactcgttgaagccgggtggcaaatgggaagtcacgaaggtgtacatgctttggaagccctggtagtcggtcttgctatccatgatgatgatcttttgtatgatcggtagcttcttttgcacgttgaggatcttttgcagccctttcttgctcacgaatacgacggtgggctggctgatgcccatgctgttcagcagctcgcgctcgttgtagatgtcgttagctggggccacagccacaccgatgaacagggcacccaacacgggcatgaagaactgcaagctattctcgctgcacaccacgatccgatggtttgtattcagcccatagcgcttcatagcttctgccagccgaacgctcatctcgaagtactcggcgtaggtaatgtccacctcgatatgtgcgtcggtaaaggcgatggtgccgggcaccagggcgtagcgcttcatggctttgtgcagctgctcgccggcggtcccgtcttcgagtgggtagaatggcgctggacccttcttaatgtttttggcatcttccataggtccagggttggactccacgtctcccgccaacttgagaaggtcaaaattcaaagtctgtttcactccgcttcccttgtacagctcgtccatgccgccggtggagtggcggccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccatggtggcgtcgactattaagtatcttgatataccgctttgtcagtttatcatgatacttaatttctagagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcgcggccgccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgctcatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggacccatgttcgccttccgccgcgtggaggaggatcacagcaacaccgagctgggcatcgtggagtaccagcacgccttcaagaccccggatgcagatgccggtgaagaaggaagcggagctactaacttcagcctgctgaagcaggctggagacgtggaggagaaccctggacctatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggacatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgatgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaagctcactgcccatgatgcagagctttcaaggataggctttattctgcaagcaatcaaataataaatctattctgctaagagatcacacatggttgtcttcagttctttttttatgtctttttaaatatatgagccacaaagggttttatgttgagggatgtgtttatgtgtatttatacatggctatgtgtgtttgtgtcatgtgcacactccacacttttttgtttacgttagatgtgggttttgatgagcaaataaaagaactaggcaataaagaaacttgtacatgggagttctgcaagtgggagtaaaaggtgcaggagaaatctggttggaagaaagacctctataggacaggactcctcagaaacagatgttttggaagagatggggaaaggttcagtgaagggggctgaacccccttccctggattgcagcacagcagcgaggaaggggctcaacgaagaaaaagtgttcctagctttaggaagtcaaggtttaggcagggatagccattctattttattaggggcaatactatttccaacggcatctggcttttctcagcccttgtgaggctctacagggaggttgaggtgttagagatcagagcaggaaacaggtttttctttccacggtaactacaatgaagtgatccttactttactaaggaacttttcattttaagtgttgacgcatgcctaaagaggtgaaattaatcccatacccttaagtctacagactggtcacagcatttcaaggaggagacctcattgtaagctactagggaggtggggacttaggtgaaggaaatgagccagcagaagctcacaagtcagcatcagcgtgtcatgtctcagcagcagaacagcacggtcagatgaaaatatagtgtgaagaatttgtataacattaattgagaaggcagattcactggagttcttatataattgaaagttaatgcacgttaataagcaagagtttagtttaatgtgatggtgttatgaacttaacgcttgtgtctccagaaaattcacatgctgaatccccaactcccaattggctccatttgtgggggaggctttggaaaagtaatcaggtttagaggggctcatgagagcagatccccatcatagaattattttcctcatcagaagcagagagattagccatttctcttccttctggtgaggacacagtgggaagtcagccaccttagggataacagggtaatgctcttcagatggacagtcagactgaagagctggcaaacagctattatgggtattatgggtgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 12 - DNA, 14985 bp, synthetic construct
  misc_feature 2103..2137: note = SEQ ID NO:14 Upstream half of beta-Actin intron
  misc_feature 2144..2398: note = 5' end of the stop cassette
  misc_feature 4295..4370: note = SEQ ID NO:15 Downstream half of beta-Actin intron
  misc_feature 7984..8124: note = Left Inverted Terminal Repeat (ITR)
  misc_feature 11067..11207: note = Right Inverted Terminal Repeat (ITR)
  source 1..14985: mol_type = other DNA; note = P439, Episomal REP/CAP + Transgene Plasmid
  misc_feature 4069..4287: note = 3' end of stop cassette
taactataacggtcctaaggtagcgaagatatccgatcaagaaagcactccgggctccagaaggagccttccaggccagctttgagcataagctgctgatgagcagtgagtgtcttgagtagtgttcagggcagcatgttaccattcatgcttgacttctagccagtgtgacgagaggctggagtcaggtctctagagagttgagcagctccagccttagatctcccagtcttatgcggtgtgcccattcgctttgtgtctgcagtcccctggccacacccagtaacagttctgggatctatgggagtagcttccttagtgagctttcccttcaaatactttgcaaccaggtagagaagtttggagtgaaggttttgttcttcgtttcttcacaatatggatatgcatcttcttttgaaaatgttaaagtaaattacctctcttttcagatactgtcttcatgcgaacttggtatcctgtttccatcccagccttctataacccagtaacatcttttttgaaaccagtgggtgagaaagacacctggtcaggaacgcggaccacaggacaactcaggctcacccacggcatcagactaaaggcaaacaaggactctgtataaagtaccggtggcatgtgtattagtggagatgcagcctgtgctctgcagacagggagtcacacagacacttttctataatttcttaagtgctttgaatgttcaagtagaaagtctaacattaaatttgattgaacaattgtatattcatggaatattttggaacggaataccaaaaaatggcaatagtggttctttctggatggaagacaaacttttcttctttaaaataaattttattttatatatttgaggttgaccacatgaccttaaggatacatatagacagtaaactggttactacagtgaagcaaattaacatatctaccatcgtacatagttacatttttttgtgtgacaggaacagctaaaatctacgtatttaacaaaactcctaaagacaatacatttttattaactatagccctcatgatgtacattagatcctctcttaaggtagcccgaataaaaatagaaaaacaacaaatgttaattttctatttttatttggtgtcgtactcaatccataatcgtcacaagcgttatagtttaagtacaacaatgtcgtcaccttgttggtgtaattaggtttacgccaacagggtgataacaggcgcgccggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggtgggtgtctttcctgcctgagctgacctgggcagacgcgtaattaagtatcatgataaactgacaaagcggtatatcaagatacttaatacatatggtcctgtaaaagtttaatgtgtaagaagtatttgttataaaagataaatattcagaatcttctttttaattcctgattttatttctataggactgaaagacttgctcgagatgtcatgaaggagatgggaggccatcacattctcacttcactgcagctcaacgtccccaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctagcgggcagtgagcgcaacgcaattaatgtgagttagctcactcattaggcaccccaggctttacactttatgcttccggctcgtatgttgtgtggaattgtgagcggataacaatttcacacaggaaacagctgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcgggttcagggggaggtgtgggaggttttttaaagcaagtaaaacctctacaaatgtggtatggctgattatgatcgcatagtgttaccatcaaccaccttaacttcatttttcttattcaatacctaggtaggtagatgctagattctggaaataaaatatgagtctcaagtggtccttgtcctctctcccagtcaaattctgaatctagttggcaagattctgaaatcaaggcatataatcagtaataagtgatgatagaagggtatatagaaggctagccgtcaacatctccactgacaaagcggtttctccaacgaattatcgattcggctgtggggtcctgtggtgtgtggggagctgtcacatccagggtcctcactgcctgtccccttccctcctcaggaggaccaggcctcatacatctccttcaatgcggcctccaactcgcggtcccaaattaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgagttctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaagggattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggtttaaacggtcctgtattagaggtcacgtgagtgttttgcgacattttgcgacaccatgtggtcacgctgggtatttaagcccgagtgagcacgcagggtctccattttgaagcgggaggtttgaacgcgcgcggccgcccgaataaaaatagaaaaacaacaaatgttaattttctatttttatttggtctagacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgctcatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggaagcggacagtgtactaattatgctctcttgaaattggctggagatgttgagagcaaccctggacctatggcgacccgcagccctggcgtcgtgattagtgatgatgaacccggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgctagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaaacgcgtgggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccatctatgtcgggtgcggagaaagaggtaataggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtagggataacagggtaatacttgaattttcaagtataaagtctagtgctaaatttaatttgaacaactgtatagtttttgctggttgggggaaggaaaaaaaatggtggcagtgtttttttcagaattagaagtgaaatgaaaacttgttgtgtgtgaggatttctaatgccatgtggtggttgcatactgagtgaagccggtgagcattctgccatgtcaccccctcgtgctcagtaatgtactttacagaaatcctaaactcaaaagattgatataaaccatgcttcttgtgtatatccggtctcttctctgggtagtctcactcagcctgcatttctgccatcccttgtcctctggcccccgtcgcccagatatctggcaaacagctattatgggtattatgggtttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaaaacacgcagatgcagtcggggcggcgcggtcccaggtccacttcgcatattaaggtgacgcgtgtggcctcgaacacagagcgacggccaccatgaccgagtacaagcccacggtgcgcctcgccacccgcgacgacgtcccccgggccgtacgcaccctcgccgccgcgttcgccgactaccccgccacgcgccacaccgttgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggattatgggtgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 13 - DNA, 4409 bp, synthetic construct
  source 1..4409: mol_type = other DNA; note = REP2/CAP9 Sequence Prior to Inserting STOP Cassette
ggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaattaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgagttctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaagggattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggtttaaacggtcctgtattagaggtcacgtgagtgttttgcgacattttgcgacaccatgtggtcacgctgggtatttaagcccgagtgagcacgcagggtctccattttgaagcgggaggtttgaacg

SEQ ID NO: 14 - DNA, 35 bp, synthetic construct
  source 1..35: mol_type = other DNA; note = Upstream half of beta-Actin Intron
gtgggtgtctttcctgcctgagctgacctgggcag

SEQ ID NO: 15 - DNA, 76 bp, synthetic construct
  source 1..76: mol_type = other DNA; note = Downstream half of beta-Actin intron
tcggctgtggggtcctgtggtgtgtggggagctgtcacatccagggtcctcactgcctgtccccttccctcctcag

SEQ ID NO: 16 - DNA, 2144 bp, synthetic construct
  source 1..2144: mol_type = other DNA; note = Transcriptional STOP cassette
aattaagtatcatgataaactgacaaagcggtatatcaagatacttaatacatatggtcctgtaaaagtttaatgtgtaagaagtatttgttataaaagataaatattcagaatcttctttttaattcctgattttatttctataggactgaaagacttgctcgagatgtcatgaaggagatgggaggccatcacattctcacttcactgcagctcaacgtccccaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctagcgggcagtgagcgcaacgcaattaatgtgagttagctcactcattaggcaccccaggctttacactttatgcttccggctcgtatgttgtgtggaattgtgagcggataacaatttcacacaggaaacagctgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcgggttcagggggaggtgtgggaggttttttaaagcaagtaaaacctctacaaatgtggtatggctgattatgatcgcatagtgttaccatcaaccaccttaacttcatttttcttattcaatacctaggtaggtagatgctagattctggaaataaaatatgagtctcaagtggtccttgtcctctctcccagtcaaattctgaatctagttggcaagattctgaaatcaaggcatataatcagtaataagtgatgatagaagggtatatagaaggctagccgtcaacatctccactgacaaagcggtttctccaacgaat

SEQ ID NO: 17 - DNA, 170 bp, synthetic construct
  source 1..170: mol_type = other DNA; note = Strong Splice Acceptor
gtcctgtaaaagtttaatgtgtaagaagtatttgttataaaagataaatattcagaatcttctttttaattcctgattttatttctataggactgaaagacttgctcgagatgtcatgaaggagatgggaggccatcacattctcacttcactgcagctcaacgtcccca

SEQ ID NO: 18 - DNA, 1672 bp, synthetic construct
  source 1..1672: mol_type = other DNA; note = Neomycin/ Kanamycin Resistance Gene
attctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctagcgggcagtgagcgcaacgcaattaatgtgagttagctcactcattaggcaccccaggctttacactttatgcttccggctcgtatgttgtgtggaattgtgagcggataacaatttcacacaggaaacagctgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcgggttcagggggaggtgtgggaggttttttaaagcaagtaaaacctctacaaatgtggtatggctgattatgatc

SEQ ID NO: 19 - DNA, 200 bp, synthetic construct
  source 1..200: mol_type = other DNA; note = transcription termination sequence from Human beta-Globin
gcatagtgttaccatcaaccaccttaacttcatttttcttattcaatacctaggtaggtagatgctagattctggaaataaaatatgagtctcaagtggtccttgtcctctctcccagtcaaattctgaatctagttggcaagattctgaaatcaaggcatataatcagtaataagtgatgatagaagggtatatagaag

SEQ ID NO: 20 - DNA, 130 bp, synthetic construct
  source 1..130: mol_type = other DNA; note = ITR, forward
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 21 - DNA, 1188 bp, synthetic construct
  source 1..1188: mol_type = other DNA; note = Human EF1-alpha Promoter, Exon1, Intron 1, and Exon2 (partial)
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgctcatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 22 - DNA, 1434 bp, synthetic construct
  source 1..1434: mol_type = other DNA; note = mCHerry-E2A-Human HPRT fusion gene
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggaagcggacagtgtactaattatgctctcttgaaattggctggagatgttgagagcaaccctggacctatggcgacccgcagccctggcgtcgtgattagtgatgatgaacccggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgctagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaa

SEQ ID NO: 23 - DNA, 272 bp, synthetic construct
  source 1..272: mol_type = other DNA; note = Poly-adenylation signal from Herpes Simplex Virus Thymidine Kinase Gene
gggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcc

SEQ ID NO: 24 - DNA, 1031 bp, synthetic construct
  source 1..1031: mol_type = other DNA; note = Human anti-repressor element 40 (AY190756.1)
gatcaagaaagcactccgggctccagaaggagccttccaggccagctttgagcataagctgctgatgagcagtgagtgtcttgagtagtgttcagggcagcatgttaccattcatgcttgacttctagccagtgtgacgagaggctggagtcaggtctctagagagttgagcagctccagccttagatctcccagtcttatgcggtgtgcccattcgctttgtgtctgcagtcccctggccacacccagtaacagttctgggatctatgggagtagcttccttagtgagctttcccttcaaatactttgcaaccaggtagagaagtttggagtgaaggttttgttcttcgtttcttcacaatatggatatgcatcttcttttgaaaatgttaaagtaaattacctctcttttcagatactgtcttcatgcgaacttggtatcctgtttccatcccagccttctataacccagtaacatcttttttgaaaccagtgggtgagaaagacacctggtcaggaacgcggaccacaggacaactcaggctcacccacggcatcagactaaaggcaaacaaggactctgtataaagtaccggtggcatgtgtattagtggagatgcagcctgtgctctgcagacagggagtcacacagacacttttctataatttcttaagtgctttgaatgttcaagtagaaagtctaacattaaatttgattgaacaattgtatattcatggaatattttggaacggaataccaaaaaatggcaatagtggttctttctggatggaagacaaacttttcttctttaaaataaattttattttatatatttgaggttgaccacatgaccttaaggatacatatagacagtaaactggttactacagtgaagcaaattaacatatctaccatcgtacatagttacatttttttgtgtgacaggaacagctaaaatctacgtatttaacaaaactcctaaagacaatacatttttattaactatagccctcatgatgtacattagatc

SEQ ID NO: 25 - DNA, 354 bp, synthetic construct
  source 1..354: mol_type = other DNA; note = Mouse anti-repressor element 40
acttgaattttcaagtataaagtctagtgctaaatttaatttgaacaactgtatagtttttgctggttgggggaaggaaaaaaaatggtggcagtgtttttttcagaattagaagtgaaatgaaaacttgttgtgtgtgaggatttctaatgccatgtggtggttgcatactgagtgaagccggtgagcattctgccatgtcaccccctcgtgctcagtaatgtactttacagaaatcctaaactcaaaagattgatataaaccatgcttcttgtgtatatccggtctcttctctgggtagtctcactcagcctgcatttctgccatcccttgtcctctggcccccgtcgccca

SEQ ID NO: 26 - DNA, 222 bp, synthetic construct
  source 1..222: mol_type = other DNA; note = Herpes Virus Thymidine Kinase Gene Promoter
ttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaaaacacgcagatgcagtcggggcggcgcggtcccaggtccacttcgcatattaaggtgacgcgtgtggcctcgaacacagagcgac

SEQ ID NO: 27 - DNA, 600 bp, synthetic construct
  source 1..600: mol_type = other DNA; note = Puromycin N-acetyl transferase coding region
atgaccgagtacaagcccacggtgcgcctcgccacccgcgacgacgtcccccgggccgtacgcaccctcgccgccgcgttcgccgactaccccgccacgcgccacaccgttgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 28 - DNA, 224 bp, synthetic construct
  source 1..224: mol_type = other DNA; note = Polyadenylation Signal from Bovine Growth Hormone Gene
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 29 - DNA, 490 bp, synthetic construct
  source 1..490: mol_type = other DNA; note = Epstein Barr Virus OriP Replication Element
cccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagg

SEQ ID NO: 30 - DNA, 1815 bp, synthetic construct
  source 1..1815: mol_type = other DNA; note = pUC57 vector sequences encoding plasmid replication origin and ampicillin resistance gene
cgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 31 - DNA, 7671 bp, synthetic construct
  source 1..7671: mol_type = other DNA; note = P321 Plasmid for Generating Recombinant Adenovirus expressing SR21 Recombinase
ttaattaaccgcaattctcatgtttgacagcttatcatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtacgtaccagtgcactggcctaggctctcttaaggtagctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcggcgcgccgccaccatggaattaaaacacattgtcaattcctataatgtgactaaaattattggctatttgagacgttccagacaagatgttgaaagagagaagcgtactggtgaagatactttgacagaacaaaaagaacttatgaataaaatattgactgggattgaaataccgtacgaaacaagaactgagatcggttctggtgaaagcattgaaggaagaccagtgtttaaaagttgcttagctgatctgaggtctggaaaatttcaagcaattgctgtaaaagaaataactcgtcttagtcgtggtagttacagtgatgctggtgaaatcgtcaatcttctaaatgaaaaaaggattatcataattacaccatataaaatatatgatccaagaaatcctgttgatgctcgtcaaattagatttgagctatttatggctagagaagaatttgaaatgacaagagaaagaatgaatggtgccaagtttacttatgctgcacaaggaaaatggatttctggtttagcaccatttggctataaattaaataagcgtacatctcgcctcgaaccatcggatgaagacaaagttgtggtaaagttgatatttgacattttcttaaatggccttgatggtaaagatttaagttatacagctattgcaactcatctttctaaacttcaattcactaccccaaggggaggaaaaagatggagtaaggacactgttagaaaaattcttcaaaatgaagcctatatgggaagggttaggtataaagcaagagaaaccacaaaagatggaaaaaaagtcttccgacctgaaagtgaacatattgttgtagatgatgcccacgaaccaataataaataaagaggactttgaagctgttcaagaaaagattaaaaataaagtgcctctgcttccagtcgtaacatcttatgaacctaatgaattagcgggaatatgtgtatgctctgtctgtggaaaatcacttcaaaaatttgaatccgaatacaataggaaaaacaaagatgggacttccagctattttcacgtgaagttattgatttgcaaaatcaacaagtgcacatcagtgagatatgagtatgttgaagaagcaattttggagtatcttgagcagttgattgctttagaaaataataaactaaaggctataattgaaaaatctatggaagcagcagaaactaacaattcagaaaagacaagtgagcaaatgctagtacaagcaaaccaaaaacagaaagaactcgaaaacaaattgacatttatttttgaaaaatttgaatctggaatttatacagatgaaatgtttttgcaaaggaaagcagctattgaaaaagaagtagctgacattaaaaaattgaaacaagaacttagcatgacatttgaagttaaagagaaagatgttaatgaatttagggttaacatatcagatgtagttaaattttataaagaatctaaaagtagaggattaaaaaatgaaaagttgagatccatttttgactttatagtattagaaatgacagagaaaagacgaggccccatccctgctaagttcaatatttatccggttttgcgaatcccgagtgtagttaattagtctagagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcgcggccgcctagatctgtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattgtt

SEQ ID NO: 32 - DNA, 20 bp, synthetic construct
  source 1..20: mol_type = other DNA; note = Primer P349F3
cgtaaacggttggtggcgca

SEQ ID NO: 33 - DNA, 24 bp, synthetic construct
  source 1..24: mol_type = other DNA; note = Primer P349R9
gtagaagggcacagtgtgggctat

SEQ ID NO: 34 - DNA, 606 bp, synthetic construct
  misc_feature 144..178: note = SEQ ID NO:14 Upstream half of beta-Actin Intron
  misc_feature 237..312: note = SEQ ID NO:15 Downstream half of beta-Actin Intron
  source 1..606: mol_type = other DNA; note = Sequence containing SEQ ID NO:14 Upstream half of beta-Actin Intron and SEQ ID NO:15 Downstream half of beta-Actin intron
cgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggtgggtgtctttcctgcctgagctgacctgggcagacgcgtaattaagtatcatgataaactgacaaagcggtttctccaacgaattatcgattcggctgtggggtcctgtggtgtgtggggagctgtcacatccagggtcctcactgcctgtccccttccctcctcaggaggaccaggcctcatacatctccttcaatgcggcctccaactcgcggtcccaaattaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaac

SEQ ID NO: 35 - DNA, 45 bp, synthetic construct
  source 1..45: mol_type = other DNA; note = AttL Recombined sequence
aattaagtatcatgataaactgacaaagcggtttctccaacgaat

SEQ ID NO: 36 - DNA, 21 bp, synthetic construct
  source 1..21: mol_type = other DNA; note = mCherry Primer 1
ctgttccacgatggtgtagtc

SEQ ID NO: 37 - DNA, 20 bp, synthetic construct
  source 1..20: mol_type = other DNA; note = mCherry Primer 2
tgaggtcaagaccacctaca

SEQ ID NO: 38 - DNA, 24 bp, synthetic construct
  source 1..24: mol_type = other DNA; note = mCherry Probe, FAM at 5' end, ZEN between bases 9 and 10, 3IABkFQ at 3' end
ttggacatcacctcccacaacgag

SEQ ID NO: 39 - DNA, 22 bp, synthetic construct
  source 1..22: mol_type = other DNA; note = Adenovirus Exon 2 (Ad5E2) Primer 1
gggtgatgcagtagaaggtaag

SEQ ID NO: 40 - DNA, 19 bp, synthetic construct
  source 1..19: mol_type = other DNA; note = Adenovirus Exon 2 (Ad5E2) Primer 2
atgaagttcggcggagatg

SEQ ID NO: 41 - DNA, 22 bp, synthetic construct
  source 1..22: mol_type = other DNA; note = Adenovirus Exon 2 (Ad5E2) Probe, HEX at 5' end, Zen between bases 9 and 10, 3IABkFQ at 3' end
tcttgttcccagcggtcccatc

SEQ ID NO: 42 - DNA, 20 bp, synthetic construct
  source 1..20: mol_type = other DNA; note = P5 (P5 Promoter region of AAV) Primer 1
gtggtcacgctgggtattta

SEQ ID NO: 43 - DNA, 22 bp, synthetic construct
  source 1..22: mol_type = other DNA; note = P5 (P5 Promoter region of AAV) Primer 2
gggaccttaatcacaatctcgt

SEQ ID NO: 44 - DNA, 23 bp, synthetic construct
  source 1..23: mol_type = other DNA; note = P5 (P5 Promoter region of AAV) Probe, FAM at 5' end, ZEN between bases 9 and 10, 31ABkFQ at 3' end
tttgaagcgggaggtttgaacgc

SEQ ID NO: 45 - DNA, 22 bp, synthetic construct
  source 1..22: mol_type = other DNA; note = AAV REP Gene Primer 1
gtccgtgagtgaagcagatatt

SEQ ID NO: 46 - DNA, 21 bp, synthetic construct
  source 1..21: mol_type = other DNA; note = AAV REP Gene Primer 2
ttcgatcaactacgcagacag

SEQ ID NO: 47 - DNA, 24 bp, synthetic construct
  source 1..24: mol_type = other DNA; note = AAV REP Gene Probe, FAM at 5' end, ZEN between bases 9 and 10, 3IABkFQ at 3' end
tctgatgctgtttccctgcagaca

SEQ ID NO: 48 - DNA, 21 bp, synthetic construct
  source 1..21: mol_type = other DNA; note = AAV9 CAP Gene Primer 1
ccgggtccaaggtatttgtaa

SEQ ID NO: 49 - DNA, 21 bp, synthetic construct
  source 1..21: mol_type = other DNA; note = AAV9 CAP Gene Primer 2
ctcaacccaaggcaaatcaac

SEQ ID NO: 50 - DNA, 24 bp, synthetic construct
  source 1..24: mol_type = other DNA; note = AAV9 CAP Gene Probe, FAM at 5' end, ZEN between bases 9 and 10, 3IABkFQ at 3' end
acatcaagacaacgctcgaggtct

SEQ ID NO: 51 - DNA, 21 bp, synthetic construct
  source 1..21: mol_type = other DNA; note = Beta lactamase (Ampicillin resistance) gene Primer 1
ccagaaacgctggtgaaagta

SEQ ID NO: 52 - DNA, 22 bp, synthetic construct
  source 1..22: mol_type = other DNA; note = Beta lactamase (Ampicillin resistance) gene Primer 2
ctcaaggatcttaccgctgttg

SEQ ID NO: 53 - DNA, 24 bp, synthetic construct
  source 1..24: mol_type = other DNA; note = Beta lactamase (Ampicillin resistance) gene Probe, FAM at 5' end, ZEN between bases 9 and 10, 3IABkFQ at 3' end
tgcacgagtgggttacatcgaact

SEQ ID NO: 54 - DNA, 14 bp, synthetic construct
  source 1..14: mol_type = other DNA; note = Bacillus safensis strain CCMA-560
actgacaaagcggt

SEQ ID NO: 55 - DNA, 863 bp, synthetic construct
  source 1..863: mol_type = other DNA; note = 5' portion of the AAV2 rep gene upstream of the stop cassette in the exemplified construct
ggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccag

SEQ ID NO: 56 - DNA, 1150 bp, synthetic construct
  source 1..1150: mol_type = other DNA; note = 3' portion of the AAV2 rep gene downstream of the stop cassette in the exemplified construct
gaggaccaggcctcatacatctccttcaatgcggcctccaactcgcggtcccaaattaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgagttctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggt

SEQ ID NO: 57 - DNA, 2399 bp, synthetic construct
  source 1..2399: mol_type = other DNA; note = the AAV9 cap gene included in the exemplified construct, including the polyadenylation signal of AAV2 having nucleotide numbers 4411 to 4466 of the seq of GenBank NC_001401.2, and an AAV2 rep P5 promoter having nucleotide 190 to to 313 of the seq of
atggctgccgatggttatcttccagattggctcgaggacaaccttagtgaagggattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggtttaaacggtcctgtattagaggtcacgtgagtgttttgcgacattttgcgacaccatgtggtcacgctgggtatttaagcccgagtgagcacgcagggtctccattttgaagcgggaggtttgaacgcgc

SEQ ID NO: 58 - DNA, 600 bp, synthetic construct
  source 1..600: mol_type = other DNA; note = CCMA-560 DNA sequence (query) from Fig. 2
tgactttatagtattagaaatgacagagaaaagacgaggccccatccctgctaagttcaatatttatccggttttgcgaattccgagtgtagttaattaagtatcatgataaactgacaaagcggtttctccaacgaatgccatgggtgcaacaaaattgatttcagaaaaactatttttccaagcaaacgaaagtattccgaatcaaaaaacgaggttttgctctgtacgctttggcaatgtgcttggatctagaggttccgttattccgatcatgctccagcagctattaaatgaaaaacctttgaccgtgactgatcctcatatgacacgtttttttatgtccattgaagaggctgtttccctcacacttcaagcagcaatcatgatgaaaggcggcgaaaccttcattctcaagatggagtccttacagcttgccgatctcctaaaagcgtttcatgaatatgccgctcaaatcaatgctaaatctccggatattcttgtagtcggaaaaagacctggcgaaaagcttcacgaggagctcacatttccgcacgaagcagatgcactgtttgaacatgaacaattttatgccatttt

SEQ ID NO: 59 - DNA, 488 bp, synthetic construct
  source 1..488: mol_type = other DNA; note = nucleotide 464352 to 464839 of whole genome shotgun sequence of Bacillus safensis strain Fairview contig56_1 (Sbjct) from Fig. 2
actgacaaagcggtttctccaacgaatgccatgggtgcaacaaaattgatttcagaaaaactatttttccaagcaaacgaaagtattccaaataaaaaaaccaagttttgctctgtacgctttggcaatgtgcttggatctagaggttccgtgattccgatcatgctccagcagctattaaatgaaaaacctttgaccgtgactgatcctcatatgacacgtttttttatgtccattgaagaggctgtttccctcacacttcaagcagcaatcatgatgaaaggcggcgaaaccttcattctcaagatggagtccttacagcttgccgatctcctaaaagcgtttcatgaatatgccgctcaaatcaatgctaaatctccggatattcttgtagtcggaaaaagacctggcgaaaagcttcacgaggagctcacatttccgcacgaagcagatgcactgtttgaacatgaacaattttatgccatttt

SEQ ID NO: 60 - DNA, 225 bp, synthetic construct
  source 1..225: mol_type = other DNA; note = SV40 polyadenylation signal
gatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcgggttcagggggaggtgtgggaggttttttaaagcaagtaaaacctctacaaatgtggtatggctgattatgatc

SEQ ID NO: 61 - DNA, 4281 bp, synthetic construct
  source 1..4281: mol_type = other DNA; note = the modified rep gene, including 5'rep, intron 5', stop cassette,intron 3' and 3' rep
ggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggtgggtgtctttcctgcctgagctgacctgggcagacgcgtaattaagtatcatgataaactgacaaagcggtatatcaagatacttaatacatatggtcctgtaaaagtttaatgtgtaagaagtatttgttataaaagataaatattcagaatcttctttttaattcctgattttatttctataggactgaaagacttgctcgagatgtcatgaaggagatgggaggccatcacattctcacttcactgcagctcaacgtccccaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctagcgggcagtgagcgcaacgcaattaatgtgagttagctcactcattaggcaccccaggctttacactttatgcttccggctcgtatgttgtgtggaattgtgagcggataacaatttcacacaggaaacagctgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcgggttcagggggaggtgtgggaggttttttaaagcaagtaaaacctctacaaatgtggtatggctgattatgatcgcatagtgttaccatcaaccaccttaacttcatttttcttattcaatacctaggtaggtagatgctagattctggaaataaaatatgagtctcaagtggtccttgtcctctctcccagtcaaattctgaatctagttggcaagattctgaaatcaaggcatataatcagtaataagtgatgatagaagggtatatagaaggctagccgtcaacatctccactgacaaagcggtttctccaacgaattatcgattcggctgtggggtcctgtggtgtgtggggagctgtcacatccagggtcctcactgcctgtccccttccctcctcaggaggaccaggcctcatacatctccttcaatgcggcctccaactcgcggtcccaaattaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgagttctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggt

SEQ ID NO: 62 - DNA, 20 bp, synthetic construct
  source 1..20: mol_type = other DNA; note = AAVRT-F1
tgatgcgccggtgatcagat

SEQ ID NO: 63 - DNA, 24 bp, synthetic construct
  source 1..24: mol_type = other DNA; note = P349R9
gtagaagggcacagtgtgggctat

SEQ ID NO: 64 - DNA, 20 bp, synthetic construct
  source 1..20: mol_type = other DNA; note = Splice Donor Site in 5'REP
cttcagccaggtacatggag

SEQ ID NO: 65 - DNA, 20 bp, synthetic construct
  source 1..20: mol_type = other DNA; note = Mutated Splice Donor Site in 5'REP
cttcagccagatacatggag

SEQ ID NO: 66 - DNA, 2000 bp, synthetic construct
  source 1..2000: mol_type = other DNA; note = Intron Spacer
ttacgtataatacctccacgttgagaccgtacgagcgcataacggaagcgttgttgagacccagtcgcctgtgataccgccttggttaaggcccgtagcctagcttaaggcaacgaaacaagacgactcaggagaatcattatgaacagggcgcgcgggaggtcgtggatatggccggtattttacttatgggaggatcttttggccgcgagacttccgagaaaccatcaacagttcgatttcctccttttatccttaacatagcacagagttgccacattgataggggggcaggagatcgtcacatgaaccgacggtcgggacatattcgatgccgtcccctctcgacaaccggattcctcgtttccaatcgaataccacgccccggggatcgtgcgcacgataagcacaataagcgtcactgcggggtcaagctgtggcttgcaggatgctaactcgtaacgacattaagacagcagagcaatgggcgacccaaaaagtttaagcctttacctcggggtggcaacataagtgtgccgactacgacaggaacccatgactcctttggggcgttctaatgggcggggtctgctgttaaccagctgatggggcaagtggaatagagcatgccctacgcgctattaaggaatgcgtggctacttggctgtgcgtacttgctcaatgtatggcaacaaacaacctaatctttgtcggcaacgcgataatctcgccacgttaatcggctacttgcatggtaggattcgattttacgtatgtaccacggattctattgcacgctacagcatcaatgtgcccgatgactactagctgcccagaggggataaatcatgtgtaatttggcgtgcattcgagttattggcattaatttctctatcaagtgcagtccctagcttcgagtaagctatgcgttcccccccgtacattttaatcccatagggaacggcacccgcacctattaagagagcgccatagctctaaacgacccacggtccaatgcttataatttctaaaatttaaggtcgcattgccttgaccgttagtccccctcacgtttgagggcataatgttcctgcgccctttacaattagtctaattctacttaaaatcgacgcaggattttttctgttggctccgctgccatgggaagctgtctatggagggctcggttatccctggcttcttactatgtaatctacctttacctttgctaattacgtgtacgtgaccagaaactacacggaacgagtgggacccataagctgagcgatggctagtgagaccgcctgatatgactcacggggtaaccgtgcaacgtgcatatatctaatgagtacggcacctttccactttgcttatatgagagtacaggctacttagcccgacgtgtacgccagatctgggcacccgccagcaggtcccactaggccggcctgccaggcaaatgacctgagtgccgcgactagcctcggccagacagctgactagcgattcacaagtgccgacttagtagttctttagcaatagaaatatagacatagttccctcgaactacagggaaatacggcctgtggttgcaagataagaggcttttaacactgacgtagtttacacactctggccggaccatgaatttcgtctgctcccacgaaccacattatcagtacttcttcttaagtgttttattaaatcgaataatctacaaatagttctaaaggagcgagttagagagaagttccacgctccataacttcgagaccgttagcgaagttttccagcacacggctacgggaccctgcactcagcttcatggttttaggcacgagaaccgcatatagtggagatttcgccgtggaagcaagtctgtcttagagagcctaggtgcttaggttttcggaactctcctgattatattcaatttccatcccattctggatcatcaagtgcccgcggatgacctacgggcaaattgccccagataa

SEQ ID NO: 67 - DNA, 2072 bp, synthetic construct
  source 1..2072: mol_type = other DNA; note = Spacer 2
gtagtcccgcagtacgcggctaggcagatcggccccttccaaatccgtgtcatcggaagaccatgttggtagtttcataatggattttattgattgctatgtaacctgaacgacggtattaatgggcttcttaacatgaacgtgagtaacggaaacattacgcttttcggtcgcgaatgacggcgaactctgggccttgtgggattatagatacgtcagtgttcctcgggaacggacaaagtaggaaatatcgacgacatggatcatgccaatatatcaacgcgggaagatgaccattgatgcagtgggaactggtgtcccacaaatatctagggcttgtgcagcggtcaagggctgtgttccacctgggacgccagcccaattatacgccagaaacgcttgattttactcaggtagaaagggggaaaatacactacgcgaccaactaataattcctttcgcaaacttaatggctgctaattttcctcaagaccggcttaatgcctactacacttaaggcgcggatggcgagatacgacgcgggcacatactaggcacgtcatcggccaggcgttctaatttaagttcacgcataaataagcgtctctcgagactgcagatccccgtcttaaatttagtacacaacgctgtttaccaccggggcgttatgacttgcagacccgtgcgtgaatatcctgtatcattcatcactgctgggactaccccactttacatatctaagggattcaacaaacttcgggtggttaatgtcaagtgcgttattggcgtataacagggtgaatgcacattgatggaatgccttcaaccaagctgtggcgtcacgccagccgaacacgttttacccaaacttagcccttttgctgcgaccctgctatctattggctatactctatatgcaatgggtgcagatcggagaccgcgccattgtcaccaccccaaggaggggagattcacgctcgctcggattagcttcccggatcatttatcaaaactggaaatactctaagctaagttcaccttcagatccttgtcaagagttgtcggcccgattagtcgttgcgcacacgtggcgtaattacagcacgcgaggtacattcgagtttatcctctgggctccattcaggcaatatgctgtcagcacgaagagtgtgacctggcgctattgttggcgacttgacgccacggaataccagtgtatatacatcctaggagaactgcaagacttcgggcgtgtatcgtaggttatggtagtcactgggatttgcacctgcgcacaatctgtttggtaaagtaaaagcgatacttcgcgactcccaataagtaacgatactgcgcggcatcccttagcgctctcataccgagcgagatacagtctatatactcaacagagcacgtttggaacctgtgaggttcagcaccaatcattcatctataaactcatcgagaaaagtatggaggccacgtgggcttcttcggttcagctaagcagactcatgagggtgtggtctcggctttggaggcagatattgtccggaattgtaattgcgactgcgcggcagtatagagtgtctctatcattgtaggactcacgcacccatcggacaaccacgtgatggccccttagttatatcgagggataggggctatgctcaagttagtcttaatttcgcgtgcttgcaaggcttatcgccggtccctcctaccgtacccggcctttcagggccatgatctttgcctgcttccctgtttatctacaccaatattgggagacttgtatgacgagcgagccactaaagaggacgattgtcaacagggtgctttatcacataatagcctgtttctccaacagtgcttcaattacctagcatcacagaatgcgataagcccaccacgtgtttcccaatggcggctattcattaatcgggacgacacccctattcgtggagagagcatcctattgagtccgagctgttagcgcatgattacggtgggccagacagattcgacggaatgccgctactacgctggcgcgtaatcagtcggaaccagaa

SEQ ID NO: 68 - DNA, 2091 bp, synthetic construct
  source 1..2091: mol_type = other DNA; note = Spacer 3
tgtacacaatgatacgatcccgtttctgcgacgtactcgggtgtagctgggaccctgtcgcatttaatcgccggtcaataggtacaccgtgctgatcttggatactcctgatcgcgatgtaacccgagcggcgctactaatgggcgtcctaacacgagcgcgaataaccacttgcgctaggcaggatttacgatggtcatctggaacggacgggtacaccatgccaaaatttgctcctagttcacaaggtcaaacggtgacccggaggccagtccactccttgtcctcgtccttacgttaccgtaacctctgacctggagcctaaggctgctccggcattttaaaaaccacacaaataaggcttcacactattcctattatcttgtgtcctacctaagaggtagtggagcgagtaggtagacacaggcactcaccaccgatgttccgcccatgggccatggttaaggtccgaatgaaacccgtatgcaagttaccggccttatattaataaggttggtatacatcctaaaagctctctcggacgtgaccaaatacaggcatccacggagcactactgaatgggcacgtggaccggcgagtgagaaatcgagcagcattaagtcattcttagtaagccgagatgggtctgcgttatgtacgtctagtggggaaacaagttggtggacaaacgttacgcctaaggagctaatatttcggtcaggttgcgctgggtcaaatcaaccggtcctggtaacggagcgatggttaggtgtattacactagcgatgggccgtgccggatggtacgccacgttctccgagccagtgaaattggatgagaactcgtaaatctctcgttgcaaacagcttcctcaatatatgacctggccactgatgagagggtaacacttaattcctagaatgtctgacgacatcacccttaagcagacacactgcgatgtgtggacgctagggtgtctctttccacgtttgacttagcccatgatgtcttgactaagatggcagggtatttccccttattcgtgtatgtaccactattaccgtcgattgaccgtcgccgcttatacgtcgtgccctgtgccttggcaattttgtccctggataacccgccttgcttctatcgaggtccttgaataagttaatgcaacttgtggaggaacatagggaatgtggcgtgctgcttagatatggagtgcaagaagtcctctgggttgtagtttcactgttacgtcctagccagtgaagcactggcatgcagctttatctgaggattccactcactgcttacaagaatttgccaagctggtgccgatatgaccccaacgctatccagagtttaagttgtcatctctctccatcataattgccacagtgtgttgtgtctacgaatagctattaaaataccagcccctgagaacgatgcccgacacatagggcagtgcaggcagcataacgtgcagatcaacataatttccactaagtgttgatatagtttactggctgagagccgccatattggaacgacgacgagagcgcgtatcctgcccaaccagaaatacgtgtccaatccatataactgaccaaagctgcggacagtttcctctccacctacaacgggattagcgcacttgcttgtagtgaggcaggtataacccacggcacgtaacacctttagacgttcggttccgcaatcgcacaatgtactactattagagaactatagcaaggaacttatagcgtatcgaaagttcatccctgcggcccatagccgtaaccaccgtggggttattttaaactttaagttgtaccctcaggcaagcgaagaccggtactttacctacatggggccgctttccgagccacgccgcaccccaacatattcctacgcgctgtaataatgaacataagagaaaatggagttgggtgacagcacatatcgactacctagcgactaccgcaacacaaagtagatcccgccggtatcctcctttcatgcaggacgggttacccaggaatgcgtttgtttgactggtgcccgctgaggcgctgaatatctcgcgatagcgtgtac

SEQ ID NO: 69 - DNA, 2794 bp, synthetic construct
  source 1..2794: mol_type = other DNA; note = mCherry-IRES-SEAP coding sequences
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagtcgactactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatcgtacggccaccatgtggggagcctgcttgctgctgctgggcctcagcctacaggtgtgcccgagcgtcatcccagtggaggaggagaatccagccttctggaaccgaaaggcagccgaggccctggatgctgccaagaagctgaagcccattcagacatcagctaagaacctcgttatcctcatgggagatgggatgggggtgtctacagtaaccgctacccggatcttaaaggggcagcagcaaggccatctgggacctgagacacagttagctatggatcgctttccacacatggctctgtccaagacatacaacacagacaagcagatcccggacagcgcaggcacaggtacagcctttctctgcggggtcaaaaccaacatgaaggtcattggcttgagtgcagctgcacgcttcaaccagtgcaacacgacatggggtaacgaggtcgtctcggtgatgcaccgtgctaagaaagcaggaaagtctgtgggagtggtgaccaccacgtcggtgcagcatgcttctccggccggcacctacgcgcacactgtgaaccgtggttggtactcggatgcacaaatgcctgcctcagcgctacaggatggctgcaaagacatctctactcagctcatctccaacatggacattgatgtgatcctcggtggtggccgcaagttcatgtttcccaaggggacaccagaccaggaatatccaactgacaccaagcaggctggaaccaggctggatggacgcaacctagttcaagagtggctggcaaagcaccagggagcccggtatgtttggaaccgctcagagctgattcaggcatccctgaaccgatctgtgacacacctcatgggcctctttgagcctaacgacatgaaatatgaaatccaccgagaccctgctcaggacccttccctggccgagatgacggaggtggctgtgcgcatgctcagcaggaaccccaaaggcttctatctctttgtggaagggggtcgcatcgaccatggccaccatgaaactgtagcctatcgtgccctgactgaggctgtcatgttcgactcggctgttgacaaggcagacaagctcaccagtgagcaggacacgatgatccttgtcactgccgaccactctcacgtcttctcttttggtggttacacacagagaggggcttccatctttggactggctcccttcaaggctgaggatggcaaatcctttacctcgatactatacggcaacggtcctggttacaagctccataatggcgcccgggctgatgtcactgaggaagagagcagcaaccccacgtaccagcagcaggctgctgtacccctgtcgtcagagacccacagcggggaggacgtggcaatattcgcgcgtggcccacaggcgcagctggtgcacggagttcaggagcagaactacatcgcgcacgtcatggccttcgcagcctgcctggagccctacactgactgcggcctggcatcccctgctggccagagcagtgcagtgagcccgggctga

SEQ ID NO: 70 - DNA, 22126 bp, synthetic construct
  source 1..22126: mol_type = other DNA; note = P600
taactataacggtcctaaggtagcgaagatatccgatcaagaaagcactccgggctccagaaggagccttccaggccagctttgagcataagctgctgatgagcagtgagtgtcttgagtagtgttcagggcagcatgttaccattcatgcttgacttctagccagtgtgacgagaggctggagtcaggtctctagagagttgagcagctccagccttagatctcccagtcttatgcggtgtgcccattcgctttgtgtctgcagtcccctggccacacccagtaacagttctgggatctatgggagtagcttccttagtgagctttcccttcaaatactttgcaaccaggtagagaagtttggagtgaaggttttgttcttcgtttcttcacaatatggatatgcatcttcttttgaaaatgttaaagtaaattacctctcttttcagatactgtcttcatgcgaacttggtatcctgtttccatcccagccttctataacccagtaacatcttttttgaaaccagtgggtgagaaagacacctggtcaggaacgcggaccacaggacaactcaggctcacccacggcatcagactaaaggcaaacaaggactctgtataaagtaccggtggcatgtgtattagtggagatgcagcctgtgctctgcagacagggagtcacacagacacttttctataatttcttaagtgctttgaatgttcaagtagaaagtctaacattaaatttgattgaacaattgtatattcatggaatattttggaacggaataccaaaaaatggcaatagtggttctttctggatggaagacaaacttttcttctttaaaataaattttattttatatatttgaggttgaccacatgaccttaaggatacatatagacagtaaactggttactacagtgaagcaaattaacatatctaccatcgtacatagttacatttttttgtgtgacaggaacagctaaaatctacgtatttaacaaaactcctaaagacaatacatttttattaactatagccctcatgatgtacattagatcctctcttaaggtagcccgaataaaaatagaaaaacaacaaatgttaattttctatttttatttggtgtcgtactcaatccataatcgtcacaagcgttatagtttaagtacaacaatgtcgtcaccttgttggtgtaattaggtttacgccaacagggtgataacaggcgcgccggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccagatacatggagctggtcgggtggctcgtggacaaggggattacctcggagaagcagtggatccaggtgggtgtctttcctgcctgagctgacctgggcagacgcgtaattaagtatcatgataaactgacaaagcggtatatcaagatacttaatacatatggtcctgtaaaagtttaatgtgtaagaagtatttgttataaaagataaatattcagaatcttctttttaattcctgattttatttctataggactgaaagacttgctcgagatgtcatgaaggagatgggaggccatcacattctcacttcactgcagctcaacgtccccaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctagcgggcagtgagcgcaacgcaattaatgtgagttagctcactcattaggcaccccaggctttacactttatgcttccggctcgtatgttgtgtggaattgtgagcggataacaatttcacacaggaaacagctgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcgggttcagggggaggtgtgggaggttttttaaagcaagtaaaacctctacaaatgtggtatggctgattatgatcgcatagtgttaccatcaaccaccttaacttcatttttcttattcaatacctaggtaggtagatgctagattctggaaataaaatatgagtctcaagtggtccttgtcctctctcccagtcaaattctgaatctagttggcaagattctgaaatcaaggcatataatcagtaataagtgatgatagaagggtatatagaaggctagccgtcaacatctccactgacaaagcggtttctccaacgaattatcgatttacgtataatacctccacgttgagaccgtacgagcgcataacggaagcgttgttgagacccagtcgcctgtgataccgccttggttaaggcccgtagcctagcttaaggcaacgaaacaagacgactcaggagaatcattatgaacagggcgcgcgggaggtcgtggatatggccggtattttacttatgggaggatcttttggccgcgagacttccgagaaaccatcaacagttcgatttcctccttttatccttaacatagcacagagttgccacattgataggggggcaggagatcgtcacatgaaccgacggtcgggacatattcgatgccgtcccctctcgacaaccggattcctcgtttccaatcgaataccacgccccggggatcgtgcgcacgataagcacaataagcgtcactgcggggtcaagctgtggcttgcaggatgctaactcgtaacgacattaagacagcagagcaatgggcgacccaaaaagtttaagcctttacctcggggtggcaacataagtgtgccgactacgacaggaacccatgactcctttggggcgttctaatgggcggggtctgctgttaaccagctgatggggcaagtggaatagagcatgccctacgcgctattaaggaatgcgtggctacttggctgtgcgtacttgctcaatgtatggcaacaaacaacctaatctttgtcggcaacgcgataatctcgccacgttaatcggctacttgcatggtaggattcgattttacgtatgtaccacggattctattgcacgctacagcatcaatgtgcccgatgactactagctgcccagaggggataaatcatgtgtaatttggcgtgcattcgagttattggcattaatttctctatcaagtgcagtccctagcttcgagtaagctatgcgttcccccccgtacattttaatcccatagggaacggcacccgcacctattaagagagcgccatagctctaaacgacccacggtccaatgcttataatttctaaaatttaaggtcgcattgccttgaccgttagtccccctcacgtttgagggcataatgttcctgcgccctttacaattagtctaattctacttaaaatcgacgcaggattttttctgttggctccgctgccatgggaagctgtctatggagggctcggttatccctggcttcttactatgtaatctacctttacctttgctaattacgtgtacgtgaccagaaactacacggaacgagtgggacccataagctgagcgatggctagtgagaccgcctgatatgactcacggggtaaccgtgcaacgtgcatatatctaatgagtacggcacctttccactttgcttatatgagagtacaggctacttagcccgacgtgtacgccagatctgggcacccgccagcaggtcccactaggccggcctgccaggcaaatgacctgagtgccgcgactagcctcggccagacagctgactagcgattcacaagtgccgacttagtagttctttagcaatagaaatatagacatagttccctcgaactacagggaaatacggcctgtggttgcaagataagaggcttttaacactgacgtagtttacacactctggccggaccatgaatttcgtctgctcccacgaaccacattatcagtacttcttcttaagtgttttattaaatcgaataatctacaaatagttctaaaggagcgagttagagagaagttccacgctccataacttcgagaccgttagcgaagttttccagcacacggctacgggaccctgcactcagcttcatggttttaggcacgagaaccgcatatagtggagatttcgccgtggaagcaagtctgtcttagagagcctaggtgcttaggttttcggaactctcctgattatattcaatttccatcccattctggatcatcaagtgcccgcggatgacctacgggcaaattgccccagataaattcggctgtggggtcctgtggtgtgtggggagctgtcacatccagggtcctcactgcctgtccccttccctcctcaggaggaccaggcctcatacatctccttcaatgcggcctccaactcgcggtcccaaattaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgagttctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaagggattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggtttaaacggtcctgtattagaggtcacgtgagtgttttgcgacattttgcgacaccatgtggtcacgctgggtatttaagcccgagtgagcacgcagggtctccattttgaagcgggaggtttgaacgcgcgcggccgcgtagtcccgcagtacgcggctaggcagatcggccccttccaaatccgtgtcatcggaagaccatgttggtagtttcataatggattttattgattgctatgtaacctgaacgacggtattaatgggcttcttaacatgaacgtgagtaacggaaacattacgcttttcggtcgcgaatgacggcgaactctgggccttgtgggattatagatacgtcagtgttcctcgggaacggacaaagtaggaaatatcgacgacatggatcatgccaatatatcaacgcgggaagatgaccattgatgcagtgggaactggtgtcccacaaatatctagggcttgtgcagcggtcaagggctgtgttccacctgggacgccagcccaattatacgccagaaacgcttgattttactcaggtagaaagggggaaaatacactacgcgaccaactaataattcctttcgcaaacttaatggctgctaattttcctcaagaccggcttaatgcctactacacttaaggcgcggatggcgagatacgacgcgggcacatactaggcacgtcatcggccaggcgttctaatttaagttcacgcataaataagcgtctctcgagactgcagatccccgtcttaaatttagtacacaacgctgtttaccaccggggcgttatgacttgcagacccgtgcgtgaatatcctgtatcattcatcactgctgggactaccccactttacatatctaagggattcaacaaacttcgggtggttaatgtcaagtgcgttattggcgtataacagggtgaatgcacattgatggaatgccttcaaccaagctgtggcgtcacgccagccgaacacgttttacccaaacttagcccttttgctgcgaccctgctatctattggctatactctatatgcaatgggtgcagatcggagaccgcgccattgtcaccaccccaaggaggggagattcacgctcgctcggattagcttcccggatcatttatcaaaactggaaatactctaagctaagttcaccttcagatccttgtcaagagttgtcggcccgattagtcgttgcgcacacgtggcgtaattacagcacgcgaggtacattcgagtttatcctctgggctccattcaggcaatatgctgtcagcacgaagagtgtgacctggcgctattgttggcgacttgacgccacggaataccagtgtatatacatcctaggagaactgcaagacttcgggcgtgtatcgtaggttatggtagtcactgggatttgcacctgcgcacaatctgtttggtaaagtaaaagcgatacttcgcgactcccaataagtaacgatactgcgcggcatcccttagcgctctcataccgagcgagatacagtctatatactcaacagagcacgtttggaacctgtgaggttcagcaccaatcattcatctataaactcatcgagaaaagtatggaggccacgtgggcttcttcggttcagctaagcagactcatgagggtgtggtctcggctttggaggcagatattgtccggaattgtaattgcgactgcgcggcagtatagagtgtctctatcattgtaggactcacgcacccatcggacaaccacgtgatggccccttagttatatcgagggataggggctatgctcaagttagtcttaatttcgcgtgcttgcaaggcttatcgccggtccctcctaccgtacccggcctttcagggccatgatctttgcctgcttccctgtttatctacaccaatattgggagacttgtatgacgagcgagccactaaagaggacgattgtcaacagggtgctttatcacataatagcctgtttctccaacagtgcttcaattacctagcatcacagaatgcgataagcccaccacgtgtttcccaatggcggctattcattaatcgggacgacacccctattcgtggagagagcatcctattgagtccgagctgttagcgcatgattacggtgggccagacagattcgacggaatgccgctactacgctggcgcgtaatcagtcggaaccagaattaattaacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgctcatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagtcgactactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatcgtacggccaccatgtggggagcctgcttgctgctgctgggcctcagcctacaggtgtgcccgagcgtcatcccagtggaggaggagaatccagccttctggaaccgaaaggcagccgaggccctggatgctgccaagaagctgaagcccattcagacatcagctaagaacctcgttatcctcatgggagatgggatgggggtgtctacagtaaccgctacccggatcttaaaggggcagcagcaaggccatctgggacctgagacacagttagctatggatcgctttccacacatggctctgtccaagacatacaacacagacaagcagatcccggacagcgcaggcacaggtacagcctttctctgcggggtcaaaaccaacatgaaggtcattggcttgagtgcagctgcacgcttcaaccagtgcaacacgacatggggtaacgaggtcgtctcggtgatgcaccgtgctaagaaagcaggaaagtctgtgggagtggtgaccaccacgtcggtgcagcatgcttctccggccggcacctacgcgcacactgtgaaccgtggttggtactcggatgcacaaatgcctgcctcagcgctacaggatggctgcaaagacatctctactcagctcatctccaacatggacattgatgtgatcctcggtggtggccgcaagttcatgtttcccaaggggacaccagaccaggaatatccaactgacaccaagcaggctggaaccaggctggatggacgcaacctagttcaagagtggctggcaaagcaccagggagcccggtatgtttggaaccgctcagagctgattcaggcatccctgaaccgatctgtgacacacctcatgggcctctttgagcctaacgacatgaaatatgaaatccaccgagaccctgctcaggacccttccctggccgagatgacggaggtggctgtgcgcatgctcagcaggaaccccaaaggcttctatctctttgtggaagggggtcgcatcgaccatggccaccatgaaactgtagcctatcgtgccctgactgaggctgtcatgttcgactcggctgttgacaaggcagacaagctcaccagtgagcaggacacgatgatccttgtcactgccgaccactctcacgtcttctcttttggtggttacacacagagaggggcttccatctttggactggctcccttcaaggctgaggatggcaaatcctttacctcgatactatacggcaacggtcctggttacaagctccataatggcgcccgggctgatgtcactgaggaagagagcagcaaccccacgtaccagcagcaggctgctgtacccctgtcgtcagagacccacagcggggaggacgtggcaatattcgcgcgtggcccacaggcgcagctggtgcacggagttcaggagcagaactacatcgcgcacgtcatggccttcgcagcctgcctggagccctacactgactgcggcctggcatcccctgctggccagagcagtgcagtgagcccgggctgaacgcgtgggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccatctatgtcgggtgcggagaaagaggtaataggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggttaattaacctgcaggtgtacacaatgatacgatcccgtttctgcgacgtactcgggtgtagctgggaccctgtcgcatttaatcgccggtcaataggtacaccgtgctgatcttggatactcctgatcgcgatgtaacccgagcggcgctactaatgggcgtcctaacacgagcgcgaataaccacttgcgctaggcaggatttacgatggtcatctggaacggacgggtacaccatgccaaaatttgctcctagttcacaaggtcaaacggtgacccggaggccagtccactccttgtcctcgtccttacgttaccgtaacctctgacctggagcctaaggctgctccggcattttaaaaaccacacaaataaggcttcacactattcctattatcttgtgtcctacctaagaggtagtggagcgagtaggtagacacaggcactcaccaccgatgttccgcccatgggccatggttaaggtccgaatgaaacccgtatgcaagttaccggccttatattaataaggttggtatacatcctaaaagctctctcggacgtgaccaaatacaggcatccacggagcactactgaatgggcacgtggaccggcgagtgagaaatcgagcagcattaagtcattcttagtaagccgagatgggtctgcgttatgtacgtctagtggggaaacaagttggtggacaaacgttacgcctaaggagctaatatttcggtcaggttgcgctgggtcaaatcaaccggtcctggtaacggagcgatggttaggtgtattacactagcgatgggccgtgccggatggtacgccacgttctccgagccagtgaaattggatgagaactcgtaaatctctcgttgcaaacagcttcctcaatatatgacctggccactgatgagagggtaacacttaattcctagaatgtctgacgacatcacccttaagcagacacactgcgatgtgtggacgctagggtgtctctttccacgtttgacttagcccatgatgtcttgactaagatggcagggtatttccccttattcgtgtatgtaccactattaccgtcgattgaccgtcgccgcttatacgtcgtgccctgtgccttggcaattttgtccctggataacccgccttgcttctatcgaggtccttgaataagttaatgcaacttgtggaggaacatagggaatgtggcgtgctgcttagatatggagtgcaagaagtcctctgggttgtagtttcactgttacgtcctagccagtgaagcactggcatgcagctttatctgaggattccactcactgcttacaagaatttgccaagctggtgccgatatgaccccaacgctatccagagtttaagttgtcatctctctccatcataattgccacagtgtgttgtgtctacgaatagctattaaaataccagcccctgagaacgatgcccgacacatagggcagtgcaggcagcataacgtgcagatcaacataatttccactaagtgttgatatagtttactggctgagagccgccatattggaacgacgacgagagcgcgtatcctgcccaaccagaaatacgtgtccaatccatataactgaccaaagctgcggacagtttcctctccacctacaacgggattagcgcacttgcttgtagtgaggcaggtataacccacggcacgtaacacctttagacgttcggttccgcaatcgcacaatgtactactattagagaactatagcaaggaacttatagcgtatcgaaagttcatccctgcggcccatagccgtaaccaccgtggggttattttaaactttaagttgtaccctcaggcaagcgaagaccggtactttacctacatggggccgctttccgagccacgccgcaccccaacatattcctacgcgctgtaataatgaacataagagaaaatggagttgggtgacagcacatatcgactacctagcgactaccgcaacacaaagtagatcccgccggtatcctcctttcatgcaggacgggttacccaggaatgcgtttgtttgactggtgcccgctgaggcgctgaatatctcgcgatagcgtgtaccgatcaagaaagcactccgggatatctggcaaacagctattatgggtattatgggtttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaaaacacgcagatgcagtcggggcggcgcggtcccaggtccacttcgcatattaaggtgacgcgtgtggcctcgaacacagagcgacggccaccatgaccgagtacaagcccacggtgcgcctcgccacccgcgacgacgtcccccgggccgtacgcaccctcgccgccgcgttcgccgactaccccgccacgcgccacaccgttgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggattatgggtgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 71 - DNA, 19 bp, synthetic construct
  source 1..19: mol_type = other DNA; note = Beta-Actin Splice Donor
gtggatccaggtgggtgtc

SEQ ID NO: 72 - DNA, 19 bp, synthetic construct
  source 1..19: mol_type = other DNA; note = Beta-Actin Splice Acceptor
ccctcctcaggaggaccag

SEQ ID NO: 73 - DNA, 863 bp, synthetic construct
  source 1..863: mol_type = other DNA; note = Mutated 5' portion of the AAV2 rep gene upstream of the stop cassette in the exemplified construct
ggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccagatacatggagctggtcgggtggctcgtggacaaggggattacctcggagaagcagtggatccag